(19) United States
(12) Patent Application Publication
(10) Pub. No.: US 2023/0404755 A1
(43) Pub. Date: Dec. 21, 2023

(54) VALVE REPAIR DEVICE AND FIXING MECHANISM FOR CLIP PIECE ASSEMBLY THEREOF

(71) Applicant: KOKA (NANTONG) LIFESCIENCES CO., LTD., Nantong (CN)

(72) Inventors: Mingming WU, Nantong (CN); Xiaoxiao GENG, Nantong (CN); Peng ZHU, Nantong (CN); Ying LIU, Nantong (CN); Dakai CHEN, Nantong (CN)

(73) Assignee: KOKA (NANTONG) LIFESCIENCES CO., LTD., Nantong (CN)

(21) Appl. No.: 18/460,240

(22) Filed: Sep. 1, 2023

Related U.S. Application Data
(63) Continuation-in-part of application No. PCT/CN2022/081988, filed on Mar. 21, 2022.

(30) Foreign Application Priority Data
Dec. 9, 2021 (CN) 202111501187.X
Mar. 31, 2023 (CN) 202310342910.7

(51) Int. Cl. A61F 2/24

(52) U.S. Cl. CPC A61F 2/2442; A61F 2220/0016

(57) ABSTRACT

A valve repair device and a fixing mechanism for a clip piece assembly are provided. The valve repair device includes: a spacer element, the distal end thereof includes two end portion; an inner clip assembly, including a first inner clip arm and a second inner clip arm; a clip piece assembly, including a first clip piece and a second clip piece; an outer clip element, including a distal end part, a first outer clip arm, a second outer clip arm, and a clamping space; a first hinge structure; and a second hinge structure; in the state that the spacer element, the clip piece assembly and the inner clip assembly are located in the clamping space, the first outer clip arm and the second outer clip arm maintain opposite clamping forces.

### CROSS-REFERENCE TO RELATED APPLICATION(S)

This application is a continuation-in-part of International PCT Application No. PCT/CN2022/081988 filed on Mar. 21, 2022, which claims priority to Chinese Patent Application No. 202111501187.X filed on Dec. 9, 2021, and this application claims foreign priority benefits under 35 U.S.C. § 119(a)-(d) to Chinese Patent Application No. 202310342910.7 filed on Mar. 31, 2023, all three of which are hereby incorporated herein by reference in their entireties.

### TECHNICAL FIELD

The present disclosure relates to the field of medical instruments, in particular to a valve repair device which is helpful to repair a natural heart valve, and a fixing mechanism for a clip piece assembly thereof.

### BACKGROUND

In the cardiovascular system, natural heart valves (such as aortic valve, pulmonary valve, mitral valve and tricuspid valve) play a key role in ensuring the positive flow of blood for adequate supply. However, these heart valves may be damaged due to congenital malformations, inflammatory processes, infectious conditions, or diseases, which reduce their efficiency. For example, these disease processes include degenerative process (such as Barlow's disease or Fibroelastic deficiency), inflammatory process (such as rheumatic heart disease) and infectious process (such as endocarditis). In addition, the damage to the left ventricle or right ventricle caused by previous heart attack (i.e., myocardial infarction secondary to coronary artery disease) or other heart diseases (such as cardiomyopathy) can distort the geometry of the natural valve, which can result in dysfunctionality of the natural valve. The vast majority of patients undergoing valve surgery, such as mitral valve surgery, suffer from degenerative diseases, which leads to dysfunction of leaflets of the natural valve (for example, mitral valve), resulting in prolapse and blood reflux.

Reference is made to the mitral valve by way of example. The mitral regurgitation may be caused by various mechanical defects in the mitral valve or left ventricular wall. The valve leaflet, the chordae connecting the valve leaflet to the papillary muscle, or the papillary muscle itself or the left ventricular wall may be damaged or otherwise dysfunctional. Usually, the valvular annulus may be damaged, expanded or weakened, thus limiting the ability of the mitral valve to be fully closed against the greater pressure of the left ventricle, which may lead to serious cardiovascular damage or death.

For many years, the definitive treatment for such damaged valves is to perform surgical repair or replacement of valves during an open-heart surgery. However, the open-heart surgery is highly invasive and prone to many complications. Therefore, elderly and frail patients with heart valve defects are often left untreated. Recently, transcatheter techniques have been developed for introducing and implanting prosthetic devices in a much less invasive manner compared with the open-heart surgery. One of specific transcatheter techniques for accessing natural mitral and aortic valves is the transseptal technique. Transseptal technique involves inserting a catheter into the right femoral vein, and moving the catheter upwards along the inferior vena cava to enter the right atrium, then piercing the diaphragm and placing the catheter into the left atrium.

However, in the implantation treatment technology via transseptal technique, prosthetic devices such as clips or clamps usually adopt very complicated mechanical structures to capture and clamp natural valves, which puts high operational requirements on surgeons during the surgical procedure, and often leads to failure in the process of opening or turning the clips or clamps due to the complicated mechanical structure of the clips or clamps. After clamping the natural valve, the natural valve needs to be partially closed by mechanical locking. However, in view of many clinical trials, it is found that the mechanical locking/attaching mode may lead to the risk that the clips or clamps would fall off the natural valve during long-term cardiac contraction.

### SUMMARY

In view of the shortcomings of the related technologies mentioned above, the objective of the present disclosure is to provide a valve repair device and a fixing mechanism for a clip piece assembly thereof, which are used to solve the problems of inconvenient operation caused by the complicated mechanical structure design of a clip device and the risk of falling off caused by a mechanical locking/attaching mode in the related technologies.

To achieve the above and other related purposes, a first aspect of the present disclosure provides a valve repair device, including: a spacer element, a proximal end thereof is provided with a connection part connected to a delivery device and two opposite sides of a distal end thereof are provided with first hinge parts respectively; an inner clip assembly, including a first inner clip arm and a second inner clip arm, wherein proximal ends of the first inner clip arm and the second inner clip arm are hinged to the first hinge parts, respectively, and distal ends of the first inner clip arm and the second inner clip arm are provided with second hinge parts respectively; a clip piece assembly, arranged on the inner clip assembly or hinged to the first hinge part, the clip piece assembly includes a first clip piece which is opened or closed relative to the first inner clip arm and a second clip piece which is opened or closed relative to the second inner clip arm, distal ends of the first clip piece and the second clip piece are provided with a traction part respectively; an outer clip element, including a distal end part, a first outer clip arm and a second outer clip arm, the first outer clip arm and the second outer clip arm are integrally formed with the distal end part, proximal ends of the first outer clip arm and the second outer clip arm are hinged to the second hinge parts respectively; a clamping space is formed between the first outer clip arm and the second outer clip arm, in the state that the spacer element, the clip piece assembly and the inner clip assembly are located in the clamping space, the first outer clip arm and the second outer clip arm maintain opposite clamping forces.

In some embodiments, the spacer element includes a spacer body having a first stiffness; the first inner clip arm and the second inner clip arm of the inner clip assembly respectively have a second stiffness; the first outer clip arm and the second outer clip arm of the outer clip element respectively have a third stiffness.

In some embodiments, an overall stiffness of the spacer element is defined as the first stiffness; the first stiffness is greater than or equal to the second stiffness, and the second stiffness is greater than the third stiffness.

In some embodiments, an overall stiffness of the spacer element is defined as the first stiffness; the first stiffness is smaller than the second stiffness, and the second stiffness is greater than the third stiffness.

In some embodiments, the first clip piece or the second clip piece has a fourth stiffness, and the fourth stiffness is smaller than the third stiffness.

In some embodiments, an overall stiffness of the first outer clip arm or the second outer clip arm is defined as the third stiffness, and a stiffness distribution close to the proximal end of the first outer clip arm or the second outer clip arm is smaller than that close to a distal end thereof.

In some embodiments, the spacer element includes a connector head, a neck part, a first spacer plate and a second spacer plate separated from the neck part and extending to the distal end of the spacer element, and hinge structures respectively formed at distal ends of the first spacer plate and the second spacer plate, which are arranged sequentially from the proximal end to the distal end of the spacer element; wherein the connector head, the neck part, the first spacer plate, the second spacer plate, and the hinge structures are integrally formed.

In some embodiments, in the state that the spacer element, the clip piece assembly and the inner clip assembly are located in the clamping space, a height of the connector head is greater than that of the clip piece assembly or the inner clip assembly or the outer clip element.

In some embodiments, the connector head of the spacer element is fixed with the connecting part, and two opposite sides of the connecting part are respectively provided with an ear-shaped block to be snap-fitted with the delivery device; the connecting part and the connector head are respectively provided with a through hole for a driving shaft of the delivery device to penetrate therethrough.

In some embodiments, an extending direction of the two ear-shaped blocks at both sides of the connecting part is parallel to plate surfaces of the first spacer plate and the second spacer plate.

In some embodiments, the neck part includes a bridge structure for separating the first spacer plate from the second spacer plate, and a width of the bridge structure in a first direction is greater than a maximum spacing distance between the first spacer plate and the second spacer plate; a width of the bridge structure in a second direction is smaller than a maximum width of a plate body of the first spacer plate or the second spacer plate.

In some embodiments, a gap is provided between the distal ends of the first spacer plate and the second spacer plate for the driving shaft of the delivery device to pass therethrough.

In some embodiments, the distal ends of the first spacer plate and the second spacer plate are engaged with each other, and a through hole is formed at an engaged position for the driving shaft of the delivery device to penetrate therethrough.

In some embodiments, the hinge structures formed at the distal ends of the first spacer plate and the second spacer plate are two hinges which are separated from each other and curled outwardly.

In some embodiments, the hinge structure formed at the distal end of the first spacer plate or the second spacer plate is a hinge curled outwardly, wherein the hinge of the first spacer plate and the hinge of the second spacer plate are staggered with relative to each other.

In some embodiments, a maximum width of a first hinge part of the first spacer plate or a first hinge part of the second spacer plate in the first direction is not greater than a maximum width of the connecting part of the spacer element in the first direction.

In some embodiments, a width of a plate body of the first spacer plate or the second spacer plate is gradually decreased towards the hinge structure at the distal end of the first spacer plate or the second spacer plate.

In some embodiments, a thickness of a plate body of the first inner clip arm or the second inner clip arm is greater than that of the first spacer plate or the second spacer plate; the thickness of the plate body of the first inner clip arm or the second inner clip arm is greater than that of the first outer clip arm or the second outer clip arm.

In some embodiments, the proximal end of the first inner clip arm or the second inner clip arm is provided with an outwardly curled hinge; and the distal end of the first inner clip arm or the second inner clip arm is provided with an inwardly curled hinge.

In some embodiments, the hinge at the proximal end of the first inner clip arm or the second inner clip arm and the hinge at the distal end of the first inner clip arm or the second inner clip arm are staggered from each other along a direction of the plate body thereof; or, the hinge at the proximal end of the first inner clip arm or the second inner clip arm and the hinge at the distal end of the first inner clip arm or the second inner clip arm are aligned with each other along the direction of the plate body thereof.

In some embodiments, a part of the plate body close to the proximal end of the first inner clip arm or the second inner clip arm is provided with an opening for the first clip piece or the second clip piece to penetrate therethrough, so that a part of the first clip piece or the second clip piece penetrates through the opening from one side of the plate body and is engaged with the other side of the plate body.

In some embodiments, a part of the plate body close to the distal end of the first inner clip arm or the second inner clip arm is provided with a limiting block for maintaining a spacing distance between the first clip piece or the second clip piece and the first inner clip arm or the second inner clip arm.

In some embodiments, the plate body of the first inner clip arm or the second inner clip arm is provided with a fixing hole for welding or riveting the first clip piece or the second clip piece.

In some embodiments, the plate body of the first inner clip arm or the second inner clip arm is provided with a puncture hole or a puncture groove corresponding to a hangnail-shaped protrusion on the first clip piece or the second clip piece.

In some embodiments, the distal end of the first clip piece and the distal end of the second clip piece are respectively provided with a traction hole for a traction wire to pass therethrough.

In some embodiments, the traction hole is formed in a piece body of the first clip piece or the second clip piece; or, the traction hole is a perforated structure which is formed at the distal end of the first clip piece or the second clip piece and is curled outwardly.

In some embodiments, the piece body of the first clip piece or the second clip piece is provided with at least one pair of notch structures.

In some embodiments, the proximal end of the first clip piece or the second clip piece is provided with a hinge which is hinged to the first hinge part.

In some embodiments, the proximal end of the first clip piece or the second clip piece is provided with an elastic structure.

In some embodiments, a bending structure is formed close to the proximal end of the first clip piece or the second clip piece, so that a preset distance is formed between a surface of the first clip piece or the second clip piece and a surface of the first inner clip arm or the second inner clip arm.

In some embodiments, the first clip piece or the second clip piece includes: an elastic piece body with a hollowed-out structure, a tab extending from a proximal end of the elastic piece body, and a hangnail-shaped protrusion formed on a main body of the elastic piece body.

In some embodiments, the first clip piece or the second clip piece is fixed on the first inner clip arm or the second inner clip arm through the tab of the first clip piece or the second clip piece by means of welding or riveting.

In some embodiments, an included angle between the hangnail-shaped protrusion and the elastic piece body is 15°-60°.

In some embodiments, a width of a root portion of the hangnail-shaped protrusion is smaller than that of a middle portion of the hangnail-shaped protrusion.

In some embodiments, the hangnail-shaped protrusions on the first clip piece or the second clip piece are divided into one group and are close to the distal end of the first clip piece or the second clip piece.

In some embodiments, the hangnail-shaped protrusions on the first clip piece or the second clip piece are divided into multiple groups, wherein the total number of the hangnail-shaped protrusions in one of the multiple groups close to the distal end of the first clip piece or the second clip piece is greater than that in the remaining groups.

In some embodiment, the multiple groups of hangnail-shaped protrusion on the first clip piece or the second clip piece are arranged in a staggered manner.

In some embodiments, the first clip piece or the second clip piece is a double-layered elastic piece including an inner elastic piece and an outer elastic piece, the inner elastic piece is provided with the hangnail-shaped protrusions, the outer elastic piece is attached onto the inner elastic piece, and the inner elastic piece and the outer elastic piece are engaged at the proximal end of the first clip piece or the second clip piece.

In some embodiments, the outer elastic piece has a hollowed-out structure.

In some embodiments, the outer clip element is a U-shaped element, and the distal ends of the first outer clip arm and the second outer clip arm are integral structure at the distal end part of the U-shaped element.

In some embodiments, the hinge structures formed at the proximal ends of the first outer clip and the second outer clip arm are two hinges which are separated from each other and curled outwardly.

In some embodiments, the proximal end of the first outer clip arm or the second outer clip arm has the same width as that of the distal end part.

In some embodiments, the hinge structure formed at the proximal end of the first outer clip arm or the second outer clip arm is an outwardly curled hinge, and the hinge of the first outer clip arm and the hinge of the second outer clip arm are staggered with each other.

In some embodiments, the first outer clip arm or the second outer clip arm has a gradually increased width from its proximal end to the distal end part.

In some embodiments, a projection point of the hinge at the proximal end of the first outer clip arm or the second outer clip arm is fallen outside the first outer clip arm or the second outer clip arm.

In some embodiments, the first outer clip arm or the second outer clip arm has a gradually increased thickness from its proximal end to its distal end.

In some embodiments, the first outer clip arm and the second outer clip arm each extend from the distal end part towards its proximal end and include a first curved section, a second curved section and a third curved section; wherein a maximum spacing distance between the first curved sections of the first outer clip arm and the second outer clip arm is smaller than a maximum spacing distance between the second curved sections of the first outer clip arm and the second outer clip arm; and the maximum spacing distance between the first curved sections of the first outer clip arm and the second outer clip arm is greater than or equal to a minimum spacing distance between the third curved sections of the first outer clip arm and the second outer clip arm.

In some embodiments, the first outer clip arm and the second outer clip arm each extend from the distal end part towards its proximal end and include a curved section and a straight section.

In some embodiments, the distal end part of the outer clip element has a screw hole for threaded connection with the driving shaft of the delivery device.

In some embodiments, the distal end part of the outer clip element is an arc-shaped thickening structure or a base structure integrally formed at a bottom of the outer clip element.

In some embodiments, both sides of the distal end part of the outer clip element are respectively provided with a pivotal hole.

In some embodiments, the valve repair device further includes an outer frame assembly wrapped by a covering material, and the outer frame assembly includes a first outer frame and a second outer frame which are pivotally connected to two opposite sides of the distal end part of the outer clip element respectively, and the first outer frame or the second outer frame is bent into a leaflet structure so as to cover the first outer clip arm or the second outer clip arm respectively in a closed state of the outer frame assembly.

In some embodiments, the first outer frame or the second outer frame includes a pivot part for pivotally connecting to one of two opposite sides of the distal end part of the outer clip element, and a frame structure which extends from the pivot part towards the proximal end of the outer frame assembly and has a gradually increased width, wherein a proximal end of the frame structure is curved outwardly.

In some embodiments, the proximal end of the frame structure has a folding-edge structure.

In some embodiments, the valve repair device further includes a covering material for wrapping the spacer element, the inner clip assembly, the clip piece assembly, the outer clip element, and the outer frame assembly, wherein the hangnail-shaped protrusions on the clip piece assembly are exposed by penetrating through the covering material so as to capture a valve.

In some embodiments, the covering material wrapped on the spacer element and the clip piece assembly is an integrally woven material; and the covering material wrapped on the inner clip assembly, the outer clip element and the outer frame assembly is an integrally woven material.

In some embodiments, the valve repair device further includes a covering material for wrapping the spacer element, the inner clip assembly, the clip piece assembly, and the outer clip element, wherein the hangnail-shaped protrusions on the clip piece assembly are exposed by penetrating through the covering material so as to capture a valve.

In some embodiments, the covering material wrapped on the spacer element and the clip piece assembly is an integrally woven material; and the covering material wrapped on the inner clip assembly and the outer clip element is integrally woven.

A second aspect of the present disclosure further provides a fixing mechanism for a clip piece assembly. The fixing mechanism includes: a clip plate including a plate body with a preset strength, an opening formed in a proximal end of the plate body, and a limiting block formed at a distal end of the plate body; and a clip piece fixed on the clip plate, wherein the clip piece includes an elastic piece, a tab extending from a proximal end of the elastic piece for penetrating through the opening to be fixed on a rear surface of the plate body, and a hangnail-shaped protrusion formed on the elastic piece and facing towards a front surface of the plate body; wherein a distal end of the elastic piece is provided with a traction part.

In some embodiments, the plate body of the clip plate is provided with a fixing hole for welding or riveting the clip piece.

In some embodiments, the plate body of the clip plate is provided with a puncture hole or a puncture groove corresponding to the hangnail-shaped protrusion on the clip piece.

In some embodiments, the traction part at the distal end of the clip piece is a traction hole for a traction wire to penetrate therethrough.

In some embodiments, the piece body of the clip piece is provided with at least one pair of notch structures.

In some embodiments, the proximal end of the clip piece is provided with an elastic structure.

In some embodiments, a bending structure is formed close to the proximal end of the clip piece so that a preset distance is formed between a surface of the clip piece and a surface of the clip plate.

In some embodiments, an included angle between the hangnail-shaped protrusion and the elastic piece is 15°-60°.

In some embodiments, a width of a root portion of the hangnail-shaped protrusion is smaller than that of a middle portion of the hangnail-shaped protrusion.

In some embodiments, the hangnail-shaped protrusions on the clip piece are divided into one group and are close to the distal end of the clip piece.

In some embodiments, the hangnail-shaped protrusions on the clip piece are divided into multiple groups, wherein the total number of the hangnail-shaped protrusions in one of the multiple groups close to the distal end of the clip piece is greater than that in the remaining groups.

In some embodiment, the multiple groups of hangnail-shaped protrusion on the clip piece are arranged in a staggered manner.

In some embodiments, the clip piece is a double-layered elastic piece including an inner elastic piece and an outer elastic piece, the inner elastic piece is provided with the hangnail-shaped protrusions, the outer elastic piece is attached onto the inner elastic piece, and the inner elastic piece and the outer elastic piece are engaged at the proximal end of the clip piece.

In some embodiments, the outer elastic piece has a hollowed-out structure.

To sum up, the valve repair device provided by the present disclosure adopts two hinge structures to hinge the spacer element with the inner clip assembly at a first position, and hinge the inner clip assembly with the outer clip element in a U-shaped structure at a second position, respectively, which achieves better flexibility as compared with the related art in which only one hinge structure is adopted and the other position is provided with a folding structure. According to the present disclosure, it requires for less force in the case that the driving shaft drives the spacer element to move towards the distal end part of the outer clip element, which is beneficial to the precision control of surgeons during operation. Furthermore, in the present disclosure, an integrally formed U-shaped outer clip element is adopted, and the stiffness design of the outer clip arms thereof is utilized to continuously maintain opposite clamping forces for each assembly/element in the clamping space, which achieves more stable performance as compared with the related art utilizing mechanical locking/attaching mode to ensure the clamping effect. Moreover, the integrally formed U-shaped outer clip element adopted in the present disclosure provides a simpler structure and a simpler clamping mode with more stable clamping effect, as compared with the related art which provides an elastic clamping force by frames/strips. Additionally, as compared with the outer clip arms and inner clip arms formed by folding metallic woven materials in the related art, the present disclosure provides a simpler manufacturing process.

### BRIEF DESCRIPTION OF DRAWINGS

The specific features of the invention(s) to which the present disclosure relates are illustrated in the appended claims. The characteristics and advantages of the invention(s) to which the present disclosure relates can be better understood by referring to the exemplary embodiments and drawings described in details below. A brief description of the accompanying drawings is as follows:

FIG. 1 is a schematic diagram illustrating a structure of a delivery device in an embodiment of the present disclosure.

FIG. 2 is a schematic diagram illustrating a stretch state of a valve repair device in an embodiment of the present disclosure.

FIG. 3 is a schematic diagram illustrating an opened state of a valve repair device in an embodiment of the present disclosure.

FIG. 4 is a schematic diagram illustrating a closed state of a valve repair device in an embodiment of the present disclosure.

FIG. 5 is a schematic diagram illustrating an exploded structure of a spacer element in an embodiment of the present disclosure.

FIG. 6 is a schematic diagram illustrating an assembled structure of a spacer element in an embodiment of the present disclosure.

FIG. 7 is a schematic diagram illustrating a structure of a spacer element in another embodiment of the present disclosure.

FIG. 8 illustrates side views of a spacer element along two directions in an embodiment of the present disclosure.

FIG. 9 is a schematic diagram illustrating a structure of a spacer element in yet another embodiment of the present disclosure.

FIG. 10 is a schematic diagram illustrating an assembled state of an inner clip assembly in an embodiment of the present disclosure.

FIG. 11 is a schematic diagram illustrating a structure of a first or second inner clip arm in an embodiment of the present disclosure.

FIG. 12 is a schematic diagram illustrating a stretch state of a valve repair device in another embodiment of the present disclosure.

FIG. 13 is a schematic diagram illustrating a hangnail-shaped protrusion on a clip piece cooperated with a puncture hole on an inner clip arm in an embodiment of the present disclosure.

FIG. 14 is a schematic diagram illustrating a stretch state and a closed state of a clip piece assembly in an embodiment of the present disclosure.

FIG. 15 is a schematic diagram illustrating a structure of a clip piece in another embodiment of the present disclosure.

FIG. 16 is an exploded view of an assembled structure of a clip piece and an inner clip arm in an embodiment of the present disclosure.

FIG. 17 is a schematic diagram illustrating an assembled structure of a clip piece and an inner clip arm in an embodiment of the present disclosure.

FIG. 18 is a schematic diagram illustrating a structure of a hangnail-shaped protrusion on a clip piece in another embodiment of the present disclosure.

FIG. 19 is a schematic diagram illustrating a clamped state of an outer clip element in an embodiment of the present disclosure.

FIG. 20 is a schematic diagram illustrating a structure of an outer clip element in an embodiment of the present disclosure.

FIG. 21 is a schematic diagram illustrating a structure of a valve repair device in another embodiment of the present disclosure.

FIG. 22 illustrates a side view of an outer clip element in an embodiment of the present disclosure.

FIG. 23 illustrates a side view of an outer clip element in another embodiment of the present disclosure.

FIG. 24 illustrates a side view of an outer clip element in yet another embodiment of the present disclosure.

FIG. 25 is a schematic diagram illustrating an outer clip element cooperated with a driving shaft in an embodiment of the present disclosure.

FIG. 26 is a schematic diagram illustrating a structure of an outer clip element in another embodiment of the present disclosure.

FIG. 27 is a schematic diagram illustrating a structure of an outer frame assembly in a valve repair device in an embodiment of the present disclosure.

FIG. 28 is a schematic diagram illustrating a structure of an outer frame of an outer frame assembly in an embodiment of the present disclosure.

FIG. 29 is a schematic diagram illustrating a closed state of a valve repair device wrapped by a covering material in an embodiment of the present disclosure.

FIG. 30 is a schematic diagram illustrating an opened state of a valve repair device wrapped by a covering material in an embodiment of the present disclosure.

FIG. 31 is a schematic diagram illustrating an opened state of a valve repair device wrapped by a covering material in another embodiment of the present disclosure.

FIG. 32 is a schematic diagram illustrating a structure of a valve repair device provided by an embodiment of the present disclosure in a stretch state.

FIG. 33 is a schematic diagram illustrating a structure of a valve repair device provided by the embodiment of the present disclosure in an opened state.

FIG. 34 is a schematic diagram illustrating a structure of a valve repair device provided by the embodiment of the present disclosure in a closed state.

FIG. 35 is a schematic diagram illustrating a structure of a fixing mechanism for a clip piece assembly provided by an embodiment of the present disclosure.

FIG. 36 is a schematic diagram illustrating a structure of a first elastic piece and a first tab according to an embodiment of the present disclosure.

FIG. 37 is a side view of a fixing mechanism provided by an embodiment of the present disclosure which clamps a valve leaflet.

FIG. 38 is another side view of a fixing mechanism provided by an embodiment of the present disclosure which clamps a valve leaflet.

FIG. 39 is a schematic diagram illustrating a structure of a second elastic piece and a second tab provided by an embodiment of the present disclosure.

FIG. 40 is a schematic diagram illustrating a structure of a fixing mechanism provided by another embodiment of the present disclosure.

FIG. 41 is a schematic diagram illustrating a partial structure of the fixing mechanism of FIG. 40.

FIG. 42 is a schematic diagram illustrating a structure of a fixing mechanism provided by yet another embodiment of the present disclosure.

FIG. 43 is a schematic diagram illustrating a structure of a fixing mechanism provided by further another embodiment of the present disclosure.

FIG. 44 is a schematic diagram of a fixing mechanism provided by yet further another embodiment of the present disclosure.

### DETAILED DESCRIPTION

Hereinafter, implementation(s) of the present disclosure would be described by means of particularly specific embodiments, and people that are familiar with the present technology can easily understand other advantages and effects of the present disclosure from the contents disclosed in the specification.

In the following description, reference is made to the accompanying drawings, which describe several embodiments of the present disclosure. It is to be understood that other embodiments may be utilized, and modifications in terms of mechanical composition, structure(s), electricity and operation can be made without departing from the spirit and scope of the present disclosure. The detailed description below should not be considered as limiting, and the scope of the embodiments of the present disclosure is only defined by the claims of the patent as published. The terms used herein are for the purpose of describing specific embodiments only and are not intended to limit the present disclosure. Spatially related terms, such as “up”, “down”, “left”, “right”, “below”, “beneath”, “lower”, “above” and “upper”, can be used here to illustrate the relationship between one element or feature illustrated in the drawings and another.

Although terms such as first, second, etc. are used here to describe various elements or parameters in some examples, these elements or parameters should not be limited by these terms. These terms are only used to distinguish one element or parameter from another. For example, the first inner clip arm may be referred to as the second inner clip arm, and similarly, the second inner clip arm may be referred to as the first inner clip arm without departing from the scope of various embodiments as described. The first inner clip arm and the second inner clip arm each describe one inner clip arm, but they are not the one and same inner clip arm unless the context clearly indicates otherwise. Similar situations also include the first clip piece and the second clip piece, or the first outer clip arm and the second outer clip arm.

Furthermore, as used here, the singular forms “a”, “an” and “the” are intended to also include the plural forms, unless the context indicates to the contrary. It should be further understood that the terms “comprising” and “including” indicate the presence of the stated feature, step, operation, element, component, item, category and/or group, but do not exclude the presence, appearance or addition of one or more other features, steps, operations, elements, components, items, categories and/or groups. The terms “or” and “and/or” as used herein are to be interpreted as inclusive or mean any one or any combination. Therefore, “A, B or C” or “A, B and/or C” means “any of the following: A; B; C; A and B; A and C; B and C; A, B and C”. An exception to this definition occurs only in the case that a combination of elements, functions, steps or operations are inherently mutually exclusive in some way.

During the heart working, the left atrium receives oxygenated blood from the lungs. During the diastolic phase or diastole, the blood previously in the left atrium passes through the mitral valve and enters the left ventricle by means of the ventricular dilatation of the left ventricle. During the systolic phase or systole, the left ventricle contracts to force the blood to reach various organs of the body through the aortic valve and the ascending aorta. During contraction, the leaflets of the mitral valve are closed to avoid a blood reflux from the left ventricle to the left atrium, and the blood in the pulmonary vein enters the left atrium. In one exemplified embodiment, the valve repair device described in the present disclosure is used for repairing a defective mitral valve. That is to say, the valve repair device is configured to facilitate to close the leaflets of the mitral valve to avoid a blood reflux from the left ventricle to the left atrium. In another exemplary embodiment, the valve repair device described in the present disclosure is used for repairing a defective tricuspid valve.

In the present disclosure, the valve repair device can present multiple states in practical surgical application, such as a stretch state, an opened state, and a closed state. The valve repair device is implanted through a delivery catheter/sheath of a delivery device, the delivery sheath of the delivery device is inserted into the left atrium through an atrial septum, and the valve repair device is protruded out of the delivery sheath to present a stretch state; in the case that the valve repair device is delivered into the mitral valve, the portion access to the left ventricle is partially opened to present an umbrella shape; at this time, by operating the clip pieces on the inner clip arms at both sides of the valve repair device, natural valve leaflets are captured, and then the valve repair device is completely closed so as to clamp the natural mitral valve and remain itself on the natural valve leaflets.

In the present disclosure, for describing the valve repair device, the “proximal end” refers to the side of the valve repair device close to the delivery device or close to the end manipulated by the user in the case that the valve repair device is in a stretch state, and correspondingly, the “distal end” refers to the side of the valve repair device away from the delivery device or away from the end manipulated by the user in the case that the valve repair device is in the stretch state. Among others, “user” refers to a medical staff.

In the present disclosure, the “outer” in the spatial term “outer side” or “outwardly” refers to a reference direction with the axis of the spacer element as the inner central axis of the valve repair device, and the radial direction of the central axis is the “outward” direction. For example, for two surfaces of the first or second spacer plate, the surface at the side facing towards the gap between the first and second spacer plates is defined as an inner surface, and the rear surface of the inner surface is defined as an outer surface. Accordingly, in the present disclosure, a clamping space is formed between the two outer clip arms of the outer clip element, the opposite surfaces of the two outer clip arms at both sides of the clamping space are defined as inner surfaces, and a rear surface of the inner surface of each of the outer clip arms is defined as the outer surface.

In the present disclosure, the spatial term “first direction” refers to a width direction of the clamping space formed between the two outer clip arms of the outer clip element, or a width direction of the gap between the first spacer plate and the second spacer plate of the spacer element; the spatial term “second direction” refers to a direction perpendicular to the first direction. In the present disclosure, the first direction is sometimes referred to as the transverse direction, and the second direction is sometimes referred to as the longitudinal direction. For the convenience of spatial description, a third direction may also be involved herein, which is a direction perpendicular to the first direction and the second direction, so that it's sometimes referred to as the vertical direction. For example, the direction along which the spacer element moves while driven by the driving shaft is referred to as a vertical movement or up-down movement.

In the present disclosure, the spatial term “close to” refers to a position next to or adjacent to a component, and it should be understood that “close to” is defined as being at least (and including) adjacent to a given position or state.

In the present disclosure, in the case that one or more elements or components are described as being connected, combined, fixed, pivotally connected, hinged, coupled, attached or otherwise interconnected, such interconnection may be directly made between components or may be indirect, such as by using one or more intervening components. Moreover, as described herein, references to “assembly”, “component”, “member” or “portion” should not be limited to a single structural component, member or element, but may further include each component of the member, the component or the element. Moreover, as described herein, the terms “substantially” and “about” are defined as being at least close to (and including) a given value or state (preferably within 10%, more preferably within 1%, and most preferably within 0.1% of the given one).

In the present disclosure, the term “hinge” refers to a structure that realizes the connection of two components by means of hinging. In specific applications, the hinge can be a pivotal hole structure that matches with a rotating shaft, a rotating shaft that matches with a pivotal hole, or a pivoting structure; the hinge may be formed by folding or curling a sheet material.

In the present disclosure, the term “stiffness” refers to the ability of a structure to resist elastic deformation in the case that it is applied with a force. The term “stiffness” is used to characterize the difficulty of structural deformation or the external force required for the structural deformation. For example, the stiffness of a spacer body refers to the external force required for the deformation of the spacer body, the stiffness of an inner clip arm refers to the external force required for the deformation of the inner clip arm, and the stiffness of an outer clip arm refers to the external force required for the deformation of the outer clip arm. It should be understood that the stiffness of the outer clip arm is smaller than that of the inner clip arm, which means the force suffered by the outer clip arm when it is deformed is not sufficient to cause the inner clip arm to have a deformation even it's applied on the inner clip arm. The overall stiffness of the spacer element refers to the ability of the entire spacer element to resist elastic deformation when applied with a force.

In the present disclosure, the term “integrally formed” refers to a structure formed by a single process through processing techniques such as stamping, cutting, pouring, casting, etc. The entire structure is one component and is inseparable.

The valve repair device described in the present disclosure is implanted via a delivery catheter/sheath of a delivery device, the delivery sheath of the delivery device is inserted into the left atrium through an atrial septum, and the valve repair device is stretched out of the delivery sheath and presents a stretch state by controlling the driving shaft extending out of the delivery catheter/sheath; in the case that the valve repair device is delivered into the mitral valve, the portion accessed to the left ventricle is partially opened to present an umbrella shape; at this time, by operating the clip pieces on the inner clip arms at both sides of the valve repair device, natural valve leaflets are captured, and then the valve repair device is completely closed so as to clamp the natural mitral valve. Subsequently, by operating the driving shaft of the delivery device to be disengaged from the valve repair device, i.e., by operating the release structure at the distal end of the delivery catheter/delivery sheath of the delivery device, the driving shaft is pulled away to release the snap-fitted connection with the connecting part on the spacer element, and the traction wire is pulled away from the traction hole in the clip piece assembly, so that the valve repair device is remained on the natural valve leaflets, and the natural valve leaflets are hence partly clamped.

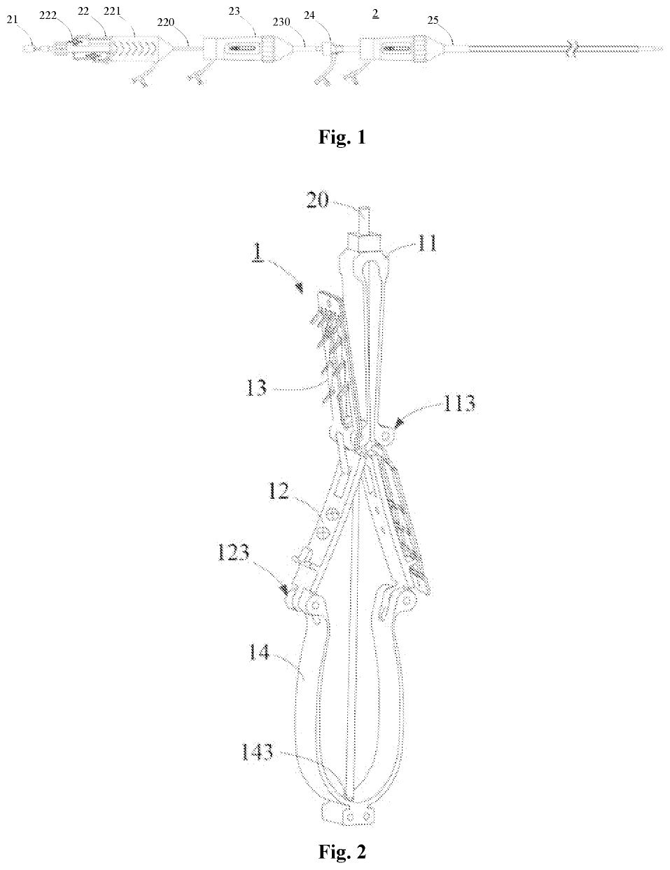

Referring to FIG. 1, which is a schematic diagram illustrating a structure of a delivery device in an embodiment of the present disclosure. As illustrated in the figure, in the present embodiment, the delivery device 2 includes a release structure 21, a delivery mechanism 22, an adjustable bending mechanism 23, a loader mechanism 24 and an outer sheath mechanism 25 from a proximal end to a distal end, sequentially. The delivery mechanism 22 is detachably connected with the valve repair device, and is used for delivering the valve repair device to a target position. The delivery mechanism 22 includes a delivery pipe 220 and a delivery handle 221. The delivery pipe 220 may pass through an adjustable elbow 230 and extend out of the adjustable elbow. The delivery handle 221 includes a delivery shell and a pulling-wire control assembly 222. A distal end of the delivery shell is connected with a proximal end of the delivery pipe 220, and a delivery sealing end is detachably arranged at an end portion of the distal end of the delivery shell. The pulling-wire control assembly 222 can slide along an axial direction of the delivery pipe on the delivery shell, and is connected with a clip piece assembly of the valve repair device. In the case that the pulling control assembly 222 slides along the axial direction, the clip piece assembly is controlled to be opened and closed, and the number of the knob(s) on the pulling control assembly 222 is as same as the number of the clip pieces in the clip piece assembly, so that one knob can control one clip piece to be opened and closed. In the present embodiment, the delivery device 2 is used in conjunction with the valve repair device. When in use, a delivery wire (pulling wire) of the delivery mechanism 22 is detachably connected with the clip piece in the clip piece assembly of the valve repair device, and a driving shaft 20 (central rod) of the release structure 21 is detachably connected with the valve repair device. In some embodiments, the valve repair device may also be referred to as a transfemoral valve repair fixture.

In an exemplified embodiment of the present disclosure, the valve repair device includes a spacer element, an inner clip assembly, a clip piece assembly, and an outer clip element; in another exemplified embodiment of the present disclosure, the valve repair device includes a spacer element, an inner clip assembly, a clip piece assembly, an outer clip element, an outer frame assembly, and a covering material.

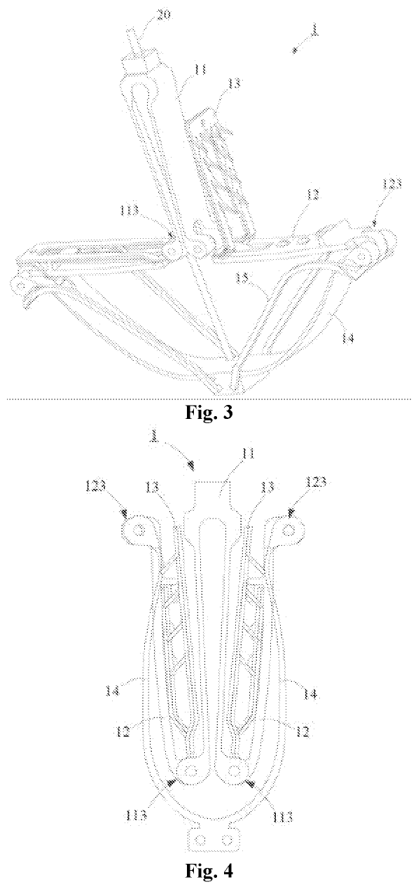

The valve repair device for example presents transitions among multiple states in practical surgical application, such as a stretch state, an opened state, and a closed state. Referring to FIG. 2 to FIG. 4, in which FIG. 2 is a schematic diagram illustrating a stretch state of a valve repair device in an embodiment of the present disclosure. FIG. 3 is a schematic diagram illustrating an opened state of a valve repair device in an embodiment of the present disclosure. FIG. 4 is a schematic diagram illustrating a closed state of a valve repair device in an embodiment of the present disclosure. As illustrated in the figures, the valve repair device 1 includes a spacer element 11, an inner clip assembly 12, a clip piece assembly 13, an outer clip element 14 and an outer frame assembly 15.

In the state illustrated in FIG. 2, the valve repair device 1 is in a stretch state. For the convenience of illustrating the internal structure, the outer frame assembly and the covering material are not illustrated in FIG. 2. In order to explain that the clip piece assembly 13 and the inner clip assembly 12 can be opened by relative rotation, one of the two clip piece assemblies 13 illustrated in FIG. 2 is in an opened state relative to the inner clip assembly 12. The stretch state refers to the state in the case that the spacer element 11 is farthest from the distal end part 143 of the outer clip element 14. In the case that the valve repair device 1 is in the stretch state, the spacer element 11, the inner clip assembly 12 (at this time, the clip piece assembly 13 is fixed on the inner clip assembly 12) and the outer clip element 14 of the valve repair device 1 are arranged sequentially from the proximal end to the distal end; in this state, the first hinge part 113 is located closer to the proximal end than the second hinge part 123. In the state illustrated in FIG. 4, that is, in the case that the valve repair device 1 is in the closed state, the spacer element 11, the inner clip assembly 12 and the clip piece assembly 13 are all folded up to be located in the clamping space of the outer clip element 14; in this state, the second hinge part 123 is located closer to the proximal end than the first hinge part 113.

In the process of the valve repair device 1 transiting from the stretch state to the closed state, it goes through an opened state, in which the spacer element 11 moves towards the distal end part 143 of the outer clip element 14; due to the movable connection between the spacer element 11 and the inner clip assembly 12 through the first hinge part 113, the inner clip assembly 12 with greater stiffness is forced to move towards both sides of the distal end part 143 by the linear movement of the spacer element 11; and due to the movable connection between the inner clip assembly 12 and the outer clip element 14 through the second hinge part 123, the outer clip element 14 with smaller stiffness is stretched out and deformed by the inner clip assembly 12; so that the whole valve repair device 1 presents an inverted umbrella-like structure, as illustrated in FIG. 3. Driven by an external force, the spacer element 11 continues to move towards the distal end part 143 of the outer clip element 14. In the case that the outer clip element 14 with smaller stiffness is opened to the maximum extent by the inner clip assembly 12, the first hinge part 113 and the second hinge part 123 are almost at the same height. As the spacer element 11 continues to move linearly towards the distal end part 143 of the outer clip element 14, the distal end of the spacer element 11 and the proximal end of the inner clip assembly 12 that are connected with the first hinge part 113 are approaching the distal end part 143 of the outer clip element 14; at this time, the inner clip assembly 12 is folded so that the proximal end thereof moves towards the distal end part 143 of the outer clip element 14, and the inner clip arms on both sides of the inner clip assembly 12 are forced to be attached onto both sides of the spacer element 11 respectively due to the elastic restoring force applied on the inner clip assembly 12 by the outer clip arms on both sides of the outer clip element 14; at this time, the valve restoration device 1 is in a closed state, as illustrated in FIG. 4.

The spacer element is configured to be positioned in the orifice of the natural valve to facilitate filling the space and forming a more effective seal, thereby reducing or preventing from the above-mentioned blood reflux. The spacer element may have a structure that is impermeable to blood and allows natural valve leaflets to be closed around the spacer element during ventricular contraction, so as to prevent blood from flowing out of the left ventricle or the right ventricle and flowing back to the left atrium or the right atrium, respectively. The spacer element is sometimes referred to as a “connecting structure” in the earlier patent application filed by the applicant, because its proximal end is used for connecting an external delivery device and its distal end is used for connecting an inner clip assembly. In the present disclosure, the spacer element can fill the space between the abnormal natural mitral leaflets or abnormal tricuspid leaflets that are incompletely closed.

The spacer body of the spacer element has a first stiffness, that is, the overall stiffness of the spacer element is defined as the first stiffness. In practical application, the spacer element is moved by a force output from the driving shaft 20 of the delivery device. In the case that the driving shaft 20 drives the spacer element to move relative to the distal end part of the outer clip element, the spacer element itself would not be deformed due to its overall design in terms of first stiffness. In another practical application, the spacer element can remain stationary, and the driving shaft 20 is fixed to the distal end part; in the case that the driving shaft 20 moves towards the delivery device, it can drive the distal end part to move towards the proximal end of the valve repair device 1; because the spacer body of the spacer element has the first stiffness, the spacer element itself would not be deformed.

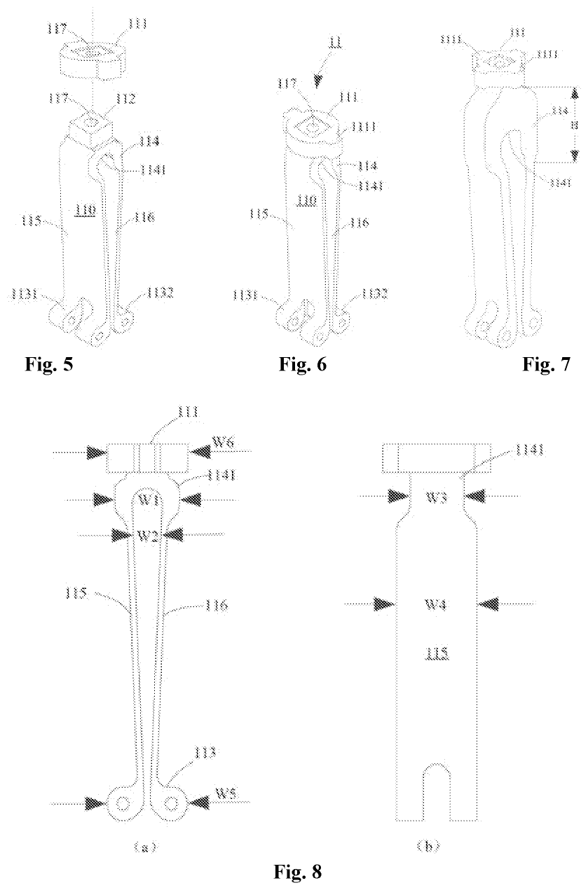

Referring to FIG. 5 and FIG. 6, in which FIG. 5 is a schematic diagram illustrating an exploded structure of the spacer element in an embodiment of the present disclosure, and FIG. 6 is a schematic diagram illustrating an assembled structure of the spacer element in an embodiment of the present disclosure. As illustrated in the figures, in the present embodiment, the spacer body 110 of the spacer element 11 has a proximal end provided with a connecting part 111 for connecting a delivery device, and a distal end having two opposite sides provided with a first hinge part, respectively. In an exemplary embodiment, the spacer element 11 includes a connector head 112, a neck part 114, a first spacer plate 115 and a second spacer plate 116 that are separated from the neck part and extending to the distal end, as well as hinge structures 1131 and 1132 that are formed at distal ends of the first spacer plate 115 and the second spacer plate 116, respectively, which are arranged from the proximal end to the distal end, sequentially. Specifically, the connector head 112, the neck part 114, the first spacer plate 115 and the second spacer plate 116, as well as the hinge structures 1131 and 1132 are integrally formed.

Referring to FIG. 4 again, in the embodiment illustrated in FIG. 4, in the state that the spacer element 11, the clip piece assembly 13 and the inner clip assembly 12 are located in the clamping space, the connector head of the spacer element 11 has a height greater than that of the clip piece assembly 13 or the inner clip assembly 12 or the outer clip element 14, so that the delivery device is not affected by any of the clip piece assembly 13, the inner clip assembly 12 and the outer clip element 14 when releasing the spacer element 11.

In an embodiment, the valve repair device is implanted through a delivery catheter/sheath of a delivery device, the delivery sheath of the delivery device is inserted into the left atrium through a septum, and the valve repair device is protruded out of the delivery sheath to present a stretch state. When the valve repair device is delivered into the mitral valve, a portion thereof access to the left ventricle is partially opened to present an inverted umbrella shape; at this time, natural valve leaflets are captured by operating the clip pieces on the inner clip arms at both sides of the valve repair device, and then the valve repair device is completely closed so as to clamp the natural mitral valve. In the case that it is confirmed that the valve repair device has clamped the natural mitral valve, it needs to be separated from the delivery catheter/delivery sheath. The delivery sheath has a release structure which can be detachably connected with the valve repair device so as to control the connection or separation between the delivery device and the valve repair device. In the present embodiment, the release structure includes a driving shaft 20 (or referred to as a central rod), a release control end and a repair control assembly, wherein a distal end of the central rod is detachably connected with the valve repair device after passing through the delivery pipe, and the connection mode between the central rod and the valve repair device is one of threaded connection and snap-fitted connection. A proximal end of the central rod is provided with the release control end, and the central rod is connected with or separated from the valve repair device through the release control end. Preferably, the release control end is in the form of a release knob, which is fixed with an end portion of the proximal end of the central rod; by rotating the release knob, the central rod is driven to rotate, so as to realize the detachable connection with or separation from the valve repair device. In order to prevent from interfering with or blocking the release structure during operation, the connector head of the spacer element has a height greater than that of the clip piece assembly or the inner clip assembly or the outer clip element, and the connecting part arranged on the connector head will also have a height greater than that of the clip piece assembly or the inner clip assembly or the outer clip element, so as to ensure that the release structure will not contact any one of the clip piece assembly, the inner clip assembly and the outer clip element during the movement of the release structure under manipulation.

In the embodiment illustrated in FIGS. 5 and 6, the connecting part 111 is fixed on the connector head 112 of the spacer element 11, and two opposite sides of the connecting part 111 are respectively provided with an ear-shaped block 1111 for snap-fitted connection with the delivery device; the connecting part 111 and the connector head 112 are respectively provided with a through hole 117 for the driving shaft 20 of the delivery device to pass therethrough.

In some embodiments, the ear-shaped block 1111 may also be referred to as a connecting ear. The connecting part 111 may also be referred to as a top seat, and the connector head 112 may be referred to as a connector. The first spacer plate 115 or the second spacer plate 116 may also be referred to as a connecting piece.

In the embodiment illustrated in FIGS. 5 and 6, the connecting part 111 is fixed on the connector head 112 by means of interference-fit connection, and two opposite sides of the connecting part 111 are respectively provided with an ear-shaped block 1111 for snap-fitted connection with the release structure of the delivery device, so that the external delivery device can be detachably connected thereto through the ear-shaped blocks 1111. Accordingly, the release structure (not illustrated in the figure) of the delivery device includes two fitting pieces, which have fitting holes corresponding to the ear-shaped blocks 1111, and the fitting piece has an elasticity and is limited on the central rod by allowing the central rod to penetrate therethrough. After the central rod is pulled away, the fitting pieces at both sides present a state of opening towards both sides due to the release of their own elasticity, thus achieving the purpose of separating the ear-shaped blocks from the fitting holes of the fitting pieces. Therefore, in order to avoid any interference or obstruction in the case that the fitting pieces are released from the elasticity and opened towards both sides, the connector head of the spacer element has a height set to be greater than that of the clip piece assembly or the inner clip assembly or the outer clip element (the height herein refers to a height of each component while the valve repair device is in the closed state).

Referring to FIG. 7, which is a schematic diagram illustrating a structure of a spacer element in another embodiment of the present disclosure. As illustrated in the figure, in the exemplary embodiment of FIG. 7, the height H of the connecting part 111 on the connector head of the spacer element 11 can be set by increasing the length of the neck part 114 of the spacer element 11.

In one embodiment, each side of the connecting part 111 of the spacer element 11 is provided with one ear-shaped block 1111; and correspondingly, the release structure of the delivery device is provided with four fitting pieces in one-to-one correspondence with the ear-shaped blocks 1111, so as to ensure that the valve repair device is arranged on the delivery catheter of the delivery device in a firmer manner. Alternatively, in another embodiment, the release structure of the delivery device includes two fitting pieces with fitting holes corresponding to the ear-shaped blocks 1111, so that when it needs to capture the valve repair device again after the release structure of the delivery device has released the valve repair device, the success rate for the two fitting pieces of the release structure to capture the connecting part 111 of the spacer element 11 can be increased, because the connecting part can be captured as long as the ear-shaped block 1111 on either of the two opposite sides is captured.

In the embodiment illustrated in FIGS. 5 and 6, the extending direction of the ear-shaped blocks 1111 at both sides of the connecting part 111 of the spacer element 11 is parallel to the plate surfaces of the first spacer plate 115 and the second spacer plate 116, so as to ensure that the outer clip arms at both sides of the outer clip element, the inner clip arms at both sides of the inner clip assembly and the two clip pieces of the clip piece assembly of the valve repair device are not located at the same sides as the two ear-shaped blocks 1111 of the connecting part while the valve repair device is in the closed state, and hence to effectively prevent from any interference or obstruction in the case that the fitting pieces are released from the elasticity and opened towards both sides.

The connecting part 111 and the connector head 112 of the spacer element 11 are provided with through holes 117 for the driving shaft 20 of the delivery device to pass therethrough, so that the driving shaft 20 of the delivery device can pass through the spacer element 11 and finally be engaged with the distal end part of the outer clip element. Correspondingly, there is a gap between distal ends of the first spacer plate 115 and the second spacer plate 116 of the spacer element 11 for the driving shaft 20 of the delivery device to pass therethrough. In another embodiment, in the case that the distal ends of the first spacer plate and the second spacer plate of the spacer element are engaged with each other, a through hole for the driving shaft 20 of the delivery device to pass therethrough is formed at the engaged position of the two distal ends.

In an embodiment, the neck part 114 of the spacer element 11 includes a bridge structure 1141 for separating the first spacer plate 115 from the second spacer plate 116. The bridge structure 1141 includes an arc surface formed on an inner wall of the connection portion to separate the first spacer plate 115 from the second spacer plate 116 by a preset distance. In the present embodiment, the bridge structure 1141 is formed as a sealing end, and a thickness of the sealing end is greater than that of the remaining portions (e.g., the first spacer plate 115 and the second spacer plate 116). In the present embodiment, a length of the sealing end is not smaller than ¼ of an overall length of the first spacer plate 115 or the second spacer plate 116. In order to prevent the two ear-shaped blocks 1111 (connecting ears), which located at the same sides as the inner clip assembly and the clip piece assembly, from hindering the withdrawal of the inner clip assembly and the clip piece assembly after the native/natural valve leaflets have been clamped, the sealing end is designed to be longer, thereby increasing the distance from the ear-shaped blocks 1111 to the inner clip assembly and the clip piece assembly.

In the present embodiment, the neck part 114 is a reinforced structure. Specifically, the bridge structure 1141 for separating the first spacer plate 115 from the second spacer plate 116 is designed to be thicker than the first spacer plate 115 and the second spacer plate 116, so as to increase the stiffness of the neck part 114; the first spacer plate 115 and the second spacer plate 116 extend from the bridge structure 1141 towards the distal end and are separated from each other at the bridge structure 1141 so as to form two pieces with a gap therebetween.

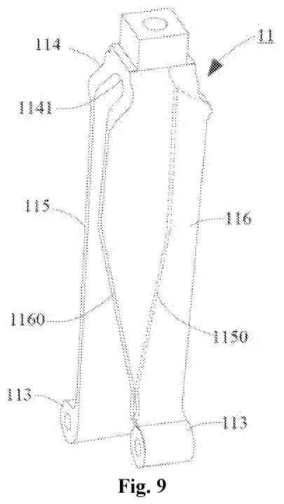

Referring to FIG. 8, which illustrates side views of the spacer element along two directions according to an embodiment of the present disclosure. As illustrated in the figure, a width W1 of the bridge structure 1141 in a first direction is greater than a maximum spacing distance W2 between the first spacer plate 115 and the second spacer plate 116; and a width W3 of the bridge structure 1141 in a second direction is smaller than a maximum width W4 of a plate body of the first spacer plate 115 or the second spacer plate 116, as illustrated in (a) and (b) of FIG. 8. In the present embodiment, the first direction is defined as the transverse direction, and the second direction is defined as the longitudinal direction; the transverse width W1 of the bridge structure 1141 is greater than the maximum spacing distance W2 between the first spacer plate 115 and the second spacer plate 116; the longitudinal width W3 of the bridge structure 1141 is smaller than the maximum width W4 of the plate body of either the first spacer plate 115 or the second spacer plate 116. With the designs above, a depressed structure can be formed at the transition between the neck part and each of the first spacer plate 115 and the second spacer plate 116, which not only strengthens the overall stiffness of the spacer element but also provides a plate surface formed at the neck part of the spacer element to facilitate the clip piece assembly, the inner clip assembly and the outer clip element to be more closely attached onto the first spacer plate 115 and the second spacer plate 116 of the spacer element in the closed state, so as to provide better sealing effect and effectively fill the space between leaflets of the natural mitral valve or tricuspid valve that is not completely closed and accordingly doesn't work normally, thereby reducing or preventing from blood reflux.

In an embodiment, the first spacer plate 115 and the second spacer plate 116 of the spacer element are separated from each other by the bridge structure 1141, and a gap between plate surfaces of the first spacer plate 115 and the second spacer plate 116 is gradually decreased from the bridge structure 1141 towards the distal ends of the first spacer plate 115 and the second spacer plate 116; that is, the gap at a position close to the bridge structure 1141 is greater than the gap between distal ends of the first spacer plate 115 and the second spacer plate 116, as illustrated in (a) of FIG. 8. In the present embodiment, the gap between distal ends of the first spacer plate 115 and the second spacer plate 116 is greater than or equal to a diameter of the driving shaft 20 of the delivery device, so that the driving shaft 20 can pass through the spacer element and reaches the distal end part of the outer clip element.

In some embodiments, a cross-section of the first spacer plate 115 or the second spacer plate 116 may be in a semicircular shape, that is, inner surfaces of the first spacer plate 115 and of the second spacer plate 116 that are opposite to each other are flat surfaces while the outer surfaces are cambered surfaces; alternatively, other cross-sectional shapes that are beneficial to realizing the engagement function are also possible.

In an embodiment, the plate body of the first spacer plate or the second spacer plate has a structure with a width gradually decreased towards the hinge structures at the distal end thereof. In the present embodiment, the width of the plate body of the first spacer plate or the second spacer plate is not uniformly designed but is gradually decreased from the proximal end to the distal end. Specifically, referring to FIG. 9, which is a schematic diagram illustrating a structure of the spacer element in another embodiment of the present disclosure. After the plate body of the first spacer plate 115 is separated from the second spacer plate 116 at the bridge structure 1141 and extends by a certain distance, one side edge (defined as the first side edge 1150, for example) of the plate body begins to retract inwardly so that the width of the plate body gradually decreases until the hinge part at the distal end. Correspondingly, after the plate body of the second spacer plate 116 is separated from the first spacer plate 115 at the bridge structure 1141 and extends by a certain distance, one side edge (for example, defined as the second side edge 1160) of the plate body begins to retract inwardly so that the width of the plate body gradually decreases until the hinge part at the distal end. By such design, a narrower or shorter hinge part can be obtained, which is beneficial to reducing the mechanical friction of the hinge part and provides more flexibility to the hinged connection between the spacer element 11 and the inner clip assembly. In the present embodiment, the plate body connected with the hinged point of the first hinge part at the distal ends of the first spacer plate 115 and the second spacer plate 116 has a smaller width and hence results in smaller friction; in this way, it requires for less pushing force for the driving shaft 20 to drive the spacer element 11 to move towards the distal end part of the outer clip element to open the outer clip arms of the outer clip element relative to each other, which makes it easier for the inner clip arms of the inner clip assembly to push the outer clip arms of the outer clip element to be opened relative to each other.

According to the above description, a part of the first side edge of the first spacer plate 115 extends with a trend of retracting inwardly, and a part of the second side edge of the second spacer plate 116 extends with a trend of retracting inwardly, so that the hinge formed on the first spacer plate 115 and the hinge of the second spacer plate 116 are staggered with each other, which ensures the stress balance of the first hinge part. In the present embodiment, the plate bodies of the two spacer plates of the spacer element are designed as asymmetric structures as described above, so that in the case that the clamping mechanism moves back and forth because the precision of the components and parts relative to each other cannot meet the requirements, the asymmetric structures will generate a resilience force to improve the mechanical stability of the whole valve repair device.

In the embodiment illustrated in FIG. 8, two opposite sides of the distal end of the spacer body of the spacer element respectively have a first hinge part 113. In the present embodiment, a maximum width W5 of the first hinge part 113 of the first spacer plate 115 and the first hinge part of the second spacer plate 116 in the first direction is not greater than a maximum width W6 of the connecting part 111 of the spacer element 11 in the first direction. In the present embodiment, the hinge structure formed at the distal end of the first spacer plate 115 or the second spacer plate 116 is an outwardly curled hinge. In the present embodiment, the maximum width W5 of the hinge of the first spacer plate 115 and the hinge of the second spacer plate 116 in the first direction is not greater than the maximum width W6 of the connecting part 111 of the spacer element 11 in the first direction, so as to limit the overall width of the proximal end and the distal end of the spacer element, which is beneficial to reducing a radial space occupied by the valve repair device in the case that the valve repair device is limited in the delivery catheter of the delivery device.

In the embodiment illustrated in (a) of FIG. 8, the outward curling of the hinge refers to curling along a direction facing away from the gap between the first spacer plate 115 and the second spacer plate 116, so that a pivot point of the first hinge part is fallen outside the main body of the spacer element, which is beneficial for the hinged connection of the inner clip assembly. Furthermore, in the case that the inner clip assembly is hinged on the first hinge part, the first inner clip arm and the second inner clip arm of the inner clip assembly would not be interfered with each other during rotation because the hinges at two sides of the first spacer plate 115 and the second spacer plate 116 are curled outwardly and have a certain distance therebetween.

In the embodiment illustrated in FIG. 5, FIG. 6 or (b) of FIG. 8, the plate bodies of the first spacer plate 115 and the second spacer plate 116 of the spacer element have uniform widths, and the hinge structures formed at the distal ends of the first spacer plate 115 and the second spacer plate 116 are two hinges separated from each other and curled outwardly, so that the proximal ends of the inner clip arms of the inner clip assembly can be hinged between the two hinges which are separated from each other and curled outwardly, and that a hinge joint with stable privation can be formed, thereby ensuring the stress balance at the hinge point.

In the embodiment illustrated in FIG. 9, the plate bodies of the first spacer plate 115 and the second spacer plate 116 of the spacer element have non-uniform widths, a part of the first side edge of the first spacer plate 115 extends with the trend of retracting inwardly, and a part of the second side edge of the second spacer plate 116 extends with the trend of retracting inwardly, so that the hinge formed on the first spacer plate 115 and the hinge of the second spacer plate 116 are staggered from each other, which ensures the stress balance of the hinge points at both sides of the spacer element.

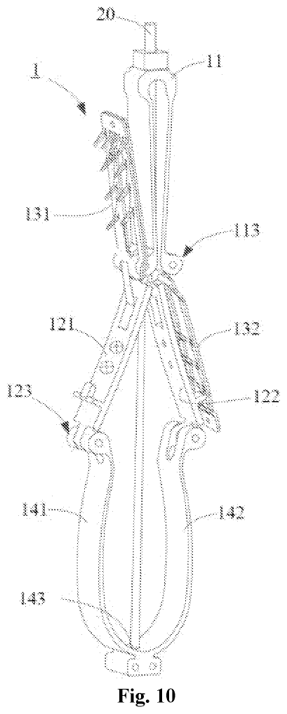

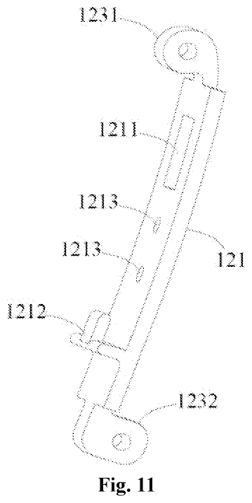

In the present disclosure, the inner clip assembly is hinged at the distal end of the spacer element by using hinge mode. Referring to FIG. 10, which is a schematic diagram illustrating an assembled state of an inner clip assembly in an embodiment of the present disclosure. As illustrated in the figure, the inner clip assembly includes a first inner clip arm 121 and a second inner clip arm 122, wherein a proximal end of the first inner clip arm 121 is hinged to the first hinge part of the first spacer plate 115, a proximal end of the second inner clip arm 122 is hinged to the first hinge part 113 of the second spacer plate 116; and the first inner clip arm 121 and the second inner clip arm 122 have the same stiffness, that is, the first inner clip arm 121 has a second stiffness, and the second inner clip arm 122 also has the second stiffness. In one embodiment, the second stiffness is smaller than or equal to the first stiffness. In another embodiment, the second stiffness is greater than the first stiffness. In the present disclosure, the second stiffness refers to an acting force applied on the proximal end and the distal end of the first inner clip arm 121 or the second inner clip arm 122 by the spacer element 11 and the outer clip element 14, and such force would not result in a deformation of the plate body of the first inner clip arm 121 or the second inner clip arm 122. The distal ends of the first inner clip arm 121 and the second inner clip arm 122 are respectively provided with a second hinge part 123.

In an embodiment, the spacer element 11, the inner clip assembly 12 and the outer clip element 14 may be made of the same material, for example, the spacer element 11, the inner clip assembly 12 and the outer clip element 14 may be obtained by cutting Nitinol material or Nickel-Titanium material with laser. In the present embodiment, the plate thickness of the first inner clip arm 121 or the second inner clip arm 122 is greater than that of the first spacer plate 115 or the second spacer plate 116 of the spacer element 11, and the plate thickness of the first inner clip arm 121 or the second inner clip arm 122 is greater than that of the first outer clip arm or the second outer clip arm of the outer clip element 14, in order to guarantee the stiffness distribution of the spacer element 11, the inner clip assembly 12 and the outer clip element 14.

Referring to FIG. 11, which is a schematic diagram illustrating a structure of a first or second inner clip arm in an embodiment of the present disclosure. As illustrated in the figure, in the present embodiment, the proximal end of the first inner clip arm 121 has a hinge 1231 which is curled outwardly, and the distal end of the first inner clip arm 121 has a hinge 1232 which is curled inwardly; correspondingly, the proximal end of the second inner clip arm 122 has a hinge 1231 which is curled outwardly, and the distal end of the second inner clip arm 122 has a hinge 1232 which is curled inwardly. Because the hinge points of the first inner clip arm 121 or the second inner clip arm 122 are distributed on different sides of the two ends of the plate body thereof, in the case that the first inner clip arm 121 and the second inner clip arm 122 are subjected to a pushing force generated by a linear movement of the spacer element 11, they can perform a force transmission more efficiently, so that the driving shaft of the delivery device requires for less pushing force and hence that the two inner clip arms of the inner clip assembly are easier to force the two outer clip arms of the outer clip element 14 to be opened.

In another embodiment, the first inner clip arm 121 or the second inner clip arm 122 may also be referred to as a clip plate, and the clip plate is a clip plate with preset strength, so that it is not easy to be deformed in the case that both ends thereof are applied with a force.

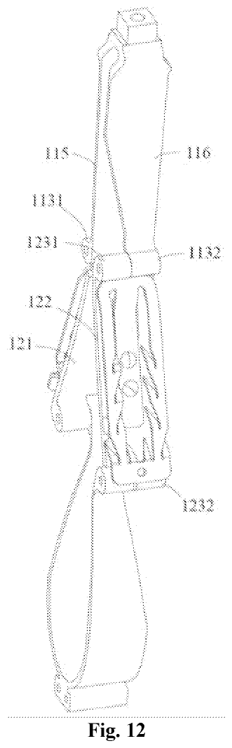

Referring to FIG. 12, which is a schematic diagram illustrating a stretch state of a valve repair device in another embodiment of the present disclosure. As illustrated in the figure, similar to the above-described case where the plate bodies of the first spacer plate 115 and the second spacer plate 116 of the spacer element 11 have non-uniform widths, and where the hinge 1131 of the first spacer plate 115 and the hinge 1132 of the second spacer plate 116 are staggered with each other, correspondingly, the hinge 1231 at the proximal end of the first inner clip arm 121 or the second inner clip arm 122 is staggered with the hinge 1232 at the distal end of the first inner clip arm 121 or the second inner clip arm 122 along a direction of the plate body, so that the proximal ends of the first inner clip arm 121 and the second inner clip arm 122 are respectively hinged to the first hinge parts 113 at the distal ends of the first spacer plate 115 and the second spacer plate 116. Because the first hinge part 113 has a relatively smaller width at the hinge point which results in less friction, the driving shaft 20 requires for less pushing force while driving the spacer element 11 to move towards the distal end part of the outer clip element 14 to open the outer clip arms of the outer clip element 14 relative to each other.

Again, similar to the case where the plate bodies of the first spacer plate 115 and the second spacer plate 116 of the spacer element 11 have uniform widths, and where the hinge structures formed at the distal ends are two hinges separated from each other and curled outwardly, correspondingly, the hinges at the proximal ends of the first inner clip arm 121 and the second inner clip arm 122 are aligned with the hinges at the distal ends of the first inner clip arm 121 and the second inner clip arm 122 along the direction of the plate bodies, so that the proximal ends of the first inner clip arms 121 and the second inner clip arm 122 of the inner clip assembly are respectively hinged to the two hinges which are separated from each other and curled outwardly; herein, the expression “be aligned with” means that the hinge at the proximal end of the first inner clip arm 121 or the second inner clip arm 122 and the hinge at the distal end of the first inner clip arm 121 or the second inner clip arm 122 are arranged on the same central axis, thus ensuring the stress balance of the hinge points.

In the present disclosure, similar to the embodiments illustrated in FIGS. 2 to 4 and the above-mentioned FIG. 12, a first clip piece 131 and a second clip piece 132 are respectively arranged on the first inner clip arm 121 and the second inner clip arm 122 of the inner clip assembly 12. In the embodiment illustrated in FIG. 11, the plate body close to the proximal end of the first inner clip arm 121 is provided with an opening 1211 for the first clip piece 131 to penetrate therethrough; specifically, the opening 1211 is configured such that a part of the first clip piece 131 pass through one side surface (such as the front surface) of the plate body of the first inner clip arm 121 to be engaged with the other side surface (such as the rear surface). Correspondingly, the plate body close to the proximal end of the second inner clip arm 122 is provided with an opening for the second clip piece 132 to pass therethrough; specifically, the opening is configured such that a part of the second clip piece 132 pass through one side surface (such as the front surface) of the plate body of the second inner clip arm 122 to be engaged with the other side surface (such as the rear surface). In the present embodiment, the plate body of the first inner clip arm 121 or the second inner clip arm 122 is provided with a fixing hole 1213 for welding or riveting the first clip piece 131 or the second clip piece 132. In the present embodiment, the first clip piece 131 and the second clip piece 132 are fixed on the first inner clip arm 121 and the second inner clip arm 122 respectively by means of welding, however, the present disclosure is not limited thereto. In some other embodiments, the first clip piece 131 and the second clip piece 132 can also be fixed on the first inner clip arm 121 and the second inner clip arm 122 respectively by means of riveting or screwing.

In the embodiment illustrated in FIG. 11, the plate bodies of the first inner clip arm 121 and the second inner clip arm 122 are respectively provided with a limiting block 1212, the limiting blocks 1212 are located close to the distal ends of the plate bodies of the first inner clip arm 121 and the second inner clip arm 122 and have a certain height. The first clip piece 131 and the second clip piece 132 are respectively arranged on the first inner clip arm 121 and the second clip piece 132 in an openable and closable manner. The cooperation between the first clip piece 131 and the first inner clip arm 121 would be described below by way of example: in the case where the first clip piece 131 is closed towards the first inner clip arm 121 to approach the surface (for example, the front surface) of the first inner clip arm 121, because the side of the first clip piece 131 corresponding to the front surface of the first inner clip arm 121 has hangnail-shaped protrusions, a certain gap needs to be preset between the first clip piece 131 and the first inner clip arm 121 to ensure that the first clip piece 131 would not be directly attached onto the front surface of the first inner clip arm 121 to compress and deform the hangnail-shaped protrusions; as a result, the height of the limiting block determines the relative distance between the first inner clip arm 121 and the first clip piece 131. Accordingly, the cooperation between the second clip piece 132 and the second inner clip arm 122 are in the same way.

In an embodiment, the certain gap preset between the first clip piece 131 and the first inner clip arm 121 is from 1 mm to 3 mm. Specifically, in some embodiments, the gap is 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm or 3 mm.

Moreover, because the first clip piece 131 and the second clip piece 132 are respectively fixed on the plate bodies of the first inner clip arm 121 and the second inner clip arm 122 by welding or riveting, it is necessary to guarantee the positioning accuracy of the fixed positions when fixing the first clip piece and the second clip piece on the plate bodies of the first inner clip arm 121 and the second inner clip arm 122 respectively as described above. The limiting blocks 1212 on the plate bodies of the first and second inner clip arms 121 and 122 play the role of positioning here, and the case that the first clip piece 131 is fixed on the first inner clip arm 121 is described by way of example: a part of the first clip piece 131 (for example, an elastic structure in the form of a tab on the first clip piece 131 would be illustrated later) penetrates through the opening in the plate body of the first inner clip arm 121 close to the proximal end thereof and reaches the other side surface, then the first clip piece 131 is fixed there by welding or riveting, and both the hangnail-shaped protrusion on the first clip piece 131 and the limiting block 1212 are located in a positioning space between the first clip piece 131 and the first inner clip arm 121; in this way, whether the first clip piece 131 is positioned accurately or not can be judged by the relative position of the hangnail-shaped protrusion on the first clip piece 131 and the limiting block 1212, so as to ensure that the first clip piece 131 can be accurately fixed at an ideal position on the first inner clip arm 121. In actual operation, the hangnail-shaped protrusion on the first clip piece 131 can be abutted against the root portion of the limiting block 1212, so as to position the first clip piece 131 at the ideal position on the first inner clip arm 121.

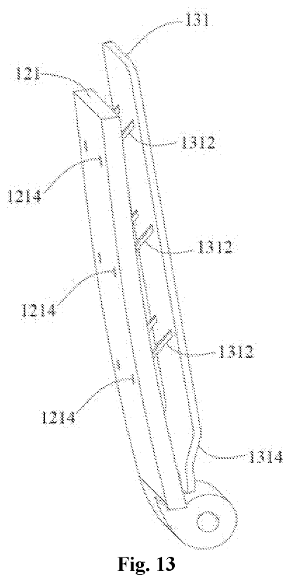

Referring to FIG. 13, which is a schematic diagram illustrating a cooperation between a hangnail-shaped protrusion on a clip piece and a puncture hole in an inner clip arm in an embodiment of the present disclosure. As illustrated in the figure, in the embodiment, the plate body of the first inner clip arm 121 or the second inner clip arm 122 is provided with a puncture hole or a puncture groove 1214 corresponding to the hangnail-shaped protrusion 1312 on the first clip piece 131 or the second clip piece 132, so that in the case that the first clip piece 131 and the second clip piece 132 are respectively closed with respect to the first inner clip arm 121 and the second inner clip arm 122 for clamping, the hangnail-shaped protrusions on the first clip piece 131 or the second clip piece 132 can penetrate into the puncture holes or the puncture grooves. Still referring to the case where the first clip piece 131 is closed with respect to the first inner clip arm 121 for clamping by way of example, in the case that the natural valve leaflets are captured between the first clip piece 131 and the first inner clip arm 121, the hangnail-shaped protrusions on the first clip piece 131 penetrate through the natural valve leaflets and then penetrate into the puncture holes or puncture grooves in the plate body of the first inner clip arm 121, so as to hold the natural valve leaflets between the first clip piece 131 and the first inner clip arm 121 more stably, and to avoid the risk that the valve repair device would fall off along with heart beating.

Referring to FIG. 10 again, as illustrated in the figure, the clip piece assembly 13 includes a first clip piece 131 and a second clip piece 132. The first clip piece 131 of the clip piece assembly has a fourth stiffness, the second clip piece 132 also has a fourth stiffness, and the fourth stiffness is smaller than the third stiffness, that is, the first clip piece 131 and the second clip piece 132 are more flexible and elastic as compared to the first outer clip arm 141 and the second outer clip arm 142 of the outer clip element 14. In some embodiments of the present disclosure, each of the first clip piece 131 and the second clip piece 132 is an integrally formed elastic piece structure. In the embodiments of the present disclosure, each of the first clip piece 131 and the second clip piece 132 is made from a sheet material of shape memory alloy, for example, by cutting Nitinol with laser. In some other embodiments of the present disclosure, each of the first clip piece 131 and the second clip piece 132 may also be a double-layered elastic piece structure.

In some embodiments, the clip piece assembly 13 is also referred to as a clamping structure. Correspondingly, the first clip piece 131 can also be referred to as a first clamping piece, and the second clip piece 132 can also be referred to as a second clamping piece.

Referring to FIG. 14, which is a schematic diagram illustrating a stretch state and a closed state of the clip piece assembly in an embodiment of the present disclosure. As illustrated in the figure, in the present embodiment, the first clip piece 131 is arranged on the first inner clip arm 121, and the distal end of the first clip piece 131 is provided with a traction part 1313; specifically, the traction part 1313 is a traction hole 1313 for a traction wire to pass therethrough. In an embodiment, the traction hole 1313 is formed in the piece body of the first clip piece 131; in another embodiment, the traction hole 1313 can also be a perforated structure formed at the distal end of the first clip piece 131 and curled outwardly. The traction hole 1313 of the first clip piece 131 is moved away relative to the first inner clip arm 121 when applied with a traction force by the traction wire 30 (in the state of the first clip piece as illustrated in FIG. 14); and correspondingly, while the traction force is removed, the first clip piece 131 is closed relative to the first inner clip arm 121 due to its own elastic restoring force (in the state of the second clip piece where the traction force is removed, as illustrated in FIG. 14). Correspondingly, the second clip piece 132 is arranged on the second inner clip arm 122, and the distal end of the second clip piece 132 is provided with a traction part 1323; specifically, the traction part 1323 is a traction hole for the traction wire 30 to pass therethrough. In an embodiment, the traction hole is formed in the piece body of the second clip piece 132; in another embodiment, the traction hole is a perforated structure formed at the distal end of the second clip piece 132 and curled outwardly. The traction hole of the second clip piece 132 is moved away relative to the second inner clip arm 122 when applied with a traction force by the traction wire 30; and correspondingly, while the traction force is removed, the second clip piece 132 is closed relative to the second inner clip arm 122 due to its own elastic restoring force. In the present embodiment, the opened angle of the first clip piece 131 or the second clip piece 132 relative to the first inner clip arm 121 or the second inner clip arm 122 is controlled by the traction force of the traction wire.

In some embodiments, the traction wire 30 is also referred to as a control thread. In an embodiment, the traction wire 30 (control thread) can also be connected with a capture ring 31, and the capture ring 31 is respectively connected with the traction wires on the first clip piece 131 and on the second clip piece 132. The traction wires 30 respectively connected with the first clip piece 131 and the second clip piece 132 are both connected to the same capture ring 31, which is used for recycling the valve repair device 1 of the present disclosure. For example, during the subsequent routine inspection, if it is found that the valve repair device 1 fails to clamp properly and needs to clamp again or needs to be withdrawn for other reasons, it is not necessary to expose the heart but just requires for delivering a snare into the heart to capture the capture ring 31, and then pull back the snare after the capture is completed, so that the first clip piece 131 and the second clip piece 132 are pulled upwards and opened to release the captured native/natural valve leaflets again; and then the snare is pulled back to receive the valve repair device 1 into the delivery sheath for recycling.

In an embodiment, the proximal end of the first clip piece 131 or the second clip piece 132 has an elastic structure, so as to ensure that an elastic restoring force can be provided to the first clip piece 131 or the second clip piece 132 in the case that the traction force at the traction hole is removed. In a specific implementation, the elastic structure can be obtained by performing one or more processes on the piece body of the clip piece, such as trimming, material reducing, bending and the like.

In an embodiment, a bending structure 1314 is formed close to the proximal end of the first clip piece 131 or the second clip piece 132 so as to provide a preset distance between the surface of the first clip piece 131 or the second clip piece 132 and the surface of the first inner clip arm 121 or the second inner clip arm 122. The bending structure 1314 is also used as the elastic structure to provide the elastic restoring force for the clip piece, which is illustrated as the structure in FIG. 13.

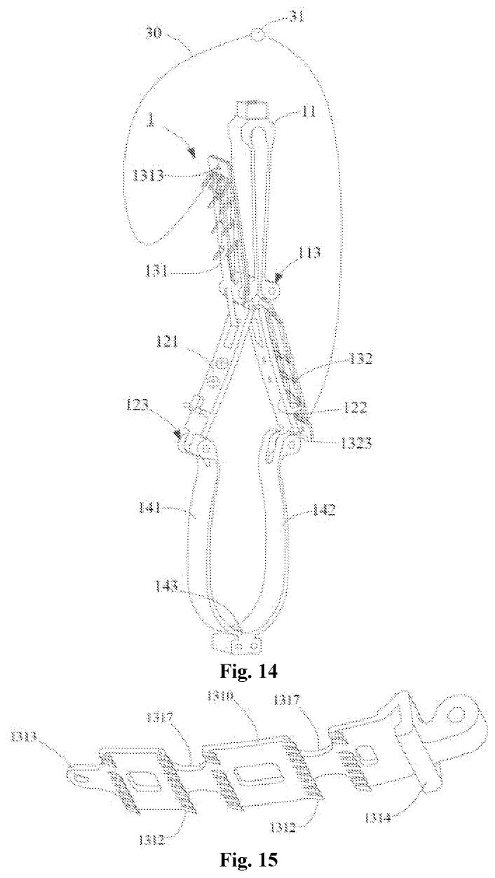

In an embodiment, the elastic piece body of the first clip piece 131 or the second clip piece 132 is a piece body with uniform width; in another embodiment, the elastic piece body is a piece body with non-uniform width. Referring to FIG. 15, which is a schematic diagram illustrating a structure of a clip piece in another embodiment of the present disclosure. As illustrated in the figure, for example, the piece body of the first clip piece 131 or the second clip piece 132 is provided with one or more pairs of notch structures (referring to the notch structure 1317 in FIG. 15); that is to say, in the present embodiment, the elastic property of the first clip piece 131 or the second clip piece 132 is enhanced by reducing the material of the piece body, and the notch structures are symmetric structures on the piece body so as to guarantee the stress balance thereof. At the same time, the design of the notch structures can also increase the friction between the first clip piece 131 or the second clip piece 132 and the valve leaflets, and prevent the valve leaflets from escaping during the clamping process.

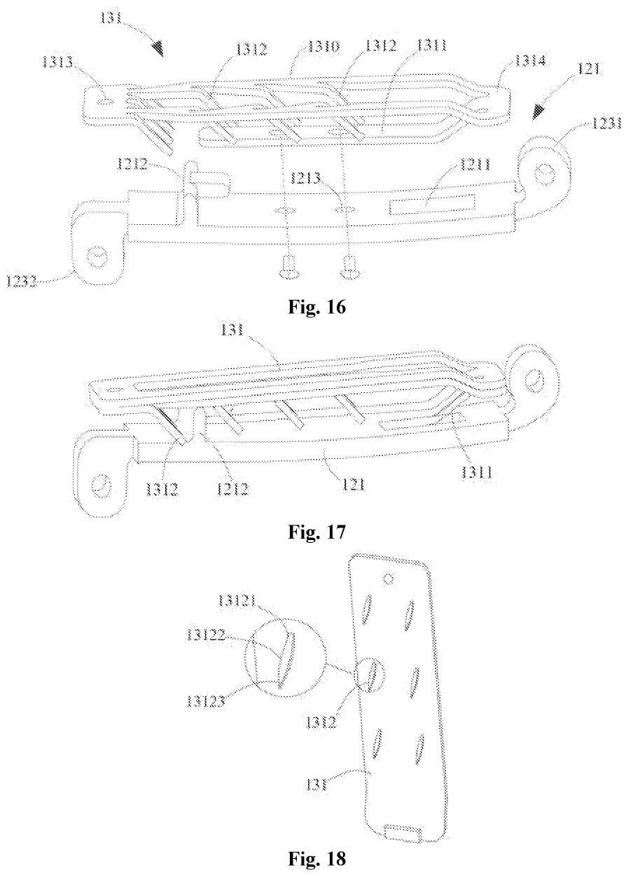

Referring to FIG. 16, which is an exploded view of an assembled structure of a clip piece and an inner clip arm in an embodiment of the present disclosure. As illustrated in the figure, the first clip piece 131 and the second clip piece 132 are respectively fixed on the plate bodies of the first inner clip arm 121 and the second inner clip arm 122 by welding or riveting. In the above embodiment, each of the first clip piece 131 and the second clip piece 132 includes an elastic piece body 1310 with a hollowed-out structure, a tab 1311 extending from a proximal end of the elastic piece body, and a hangnail-shaped protrusion 1312 formed on a main body of the elastic piece body 1310. Specifically, the elastic piece body 1310, the tab 1311 and the hangnail-shaped protrusion 1312 are all integrally formed on the first clip piece 131 or the second clip piece 132, for example, by cutting, trimming or punching. In some cases, the tab 1311 can also be referred to as a fixing piece.

Referring to FIG. 17, which is a schematic diagram illustrating an assembled structure of a clip piece and an inner clip arm in an embodiment of the present disclosure. As illustrated in the figure, a tab 1311 extending from the proximal end of the elastic piece body 1310 is provided with a part of the first clip piece 131 or the second clip piece 132 close to the proximal end thereof; the tab 1311 has a certain length, and a part of the tab 1311 is welded or riveted onto the first inner clip arm 121 or the second inner clip arm 122. Still referring to the case where the first clip piece is fixed on the first inner clip arm 121 by way of example, the tab 1311 of the first clip piece passes through the opening 1211 of the first inner clip arm 121 to be engaged on the rear surface of the first inner clip arm 121, and then the tab 1311 is fixed on the rear surface of the first inner clip arm 121 by welding or riveting, while a main part (i.e., the part provided with the hangnail-shaped protrusions) of the first clip piece 131 is still located at the front surface of the first inner clip arm 121; in this way, the first clip piece 131 can be fixed on the first inner clip arm 121. Because the tab 1311 described above has certain elastic performance, it can not only fix the first clip piece 131 but also achieve the elastic engagement between the proximal end of the first clip piece 131 and the proximal end of the first inner clip arm 121, so that the first clip piece 131 is elastically closed relative to the first inner clip arm 121. Accordingly, the second clip piece 132 and the second inner clip arm 122 are cooperated with each other in the same way.

In an embodiment, the included angle between the hangnail-shaped protrusion 1312 and the elastic piece body 1310 of the first clip piece 131 or the second clip piece 132 is 15°-60°. In the present embodiment, the extending direction of the hangnail-shaped protrusion 1312 is inclined towards the proximal end of the first clip piece 131 or the second clip piece 132 by 15°-60°. Specifically, in some embodiments, the included angle is 15°, 16°, 17°, 18°, 19°, 20°, 21°, 22°, 23°, 24°, 25°, 26°, 27°, 28°, 29°, 30°, 31°, 32°, 33°, 34°, 35°, 36°, 37°, 38°, 39°, 40°, 41°, 42°, 43°, 44°, 45°, 46°, 47°, 48°, 49°, 50°, 51°, 52°, 53°, 54°, 55°, 56°, 57°, 58°, 59° or 60°. The above-mentioned angle of the hangnail-shaped protrusion 1312 provides further benefit in that the hangnail-shaped protrusion 1312 can be pulled away from the natural valve leaflets in the case that the first clip piece 131 or the second clip piece 132 can be pulled.

In an embodiment, the length of the hangnail-shaped protrusion 1312 on the first clip piece 131 or the second clip piece 132 is 0.3 mm-3 mm. Specifically, in some embodiments, the length of the hangnail-shaped protrusion 1312 is 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm or 3 mm.

Referring to FIG. 18, which is a schematic diagram illustrating a structure of a hangnail-shaped protrusion on a clip piece in another embodiment of the present disclosure. As illustrated in the figure, the width of the root portion of the hangnail-shaped protrusion on the first clip piece 131 or the second clip piece 132 is smaller than that of the middle portion; that is, each of the hangnail-shaped protrusions 131 extends from the elastic piece body at the included angle mentioned above and has a width which is smaller at the root portion 13121 and then gradually increases so as to be transited to the middle portion 13122, and then gradually decreases until a sharper needle tip 13123 is formed; in this way, the natural valve leaflets can be held in the gap between the first clip piece 131 or the second clip piece 132 and the first inner clip arm 121 or the second inner clip arm 122 in a better way in the case that the hangnail-shaped protrusion 1312 with the above-described structure passes through the natural valve leaflets.

In an embodiment, the hangnail-shaped protrusions distributed on the elastic piece body of the first clip piece 131 or the second clip piece 132 are divided into one group, which includes three hangnail-shaped protrusions or four hangnail-shaped protrusions 1312 arranged close to the distal end of the first clip piece 131 or the second clip piece 132; in another embodiment, such as the embodiment illustrated in FIGS. 13 to 18, the hangnail-shaped protrusions 1312 on the first clip piece 131 or the second clip piece 132 are divided into multiple groups, and the total number of the hangnail-shaped protrusions 1312 in the group closer to the distal end is greater than that of the remaining groups, for example, the total number of the hangnail-shaped protrusions in the group closer to the distal end is three or four, and the total number of the hangnail-shaped protrusion(s) in the remaining groups is one or two.

In an embodiment, in the case that the hangnail-shaped protrusions on the first clip piece 131 or the second clip piece 132 are divided into multiple groups, the multiple groups of hangnail-shaped protrusions are regularly arranged at intervals, for example, the interval may be regular fixed interval or arithmetic gradual interval. In an embodiment, the multiple groups of hangnail-shaped protrusions are also be arranged in a staggered manner, for example, the positions of the hangnail-shaped protrusions are arranged sequentially in a staggered manner.

In an embodiment, the first clip piece 131 or the second clip piece 132 is a double-layered elastic piece (not illustrated in the figure) including an inner elastic piece and an outer elastic piece; the hangnail-shaped protrusions are arranged on the inner elastic piece, and the outer elastic piece is attached onto the inner elastic piece to reinforce the elastic force or clamping force of the clamping piece, and the inner elastic piece is engaged with the outer elastic piece at the proximal end. In the present embodiment, the inner elastic piece can be engaged with the outer elastic piece at the proximal end by welding or riveting; alternatively, the inner elastic piece and the outer elastic piece are an integrally formed structure, which may achieve the engagement of the two elastic pieces at the proximal end by means of bending.

The first clip piece 131 is taken as an example, the first clip piece 131 has a double-layered elastic piece structure including an inner elastic piece and an outer elastic piece. The outer elastic piece is a hollowed-out structure in order to increase the elastic capacity of the outer elastic piece, and the inner elastic piece is provided with an elastic piece body with a hollowed-out structure, a tab extending from the proximal end of the elastic piece body, and hangnail-shaped protrusions formed on the main body of the elastic piece body; herein, the first clip piece 131 in the form of a double-layered elastic piece structure is fixed on the rear surface of the first inner clip arm 121 by welding or riveting the tab extending from the proximal end of the elastic piece of the inner elastic piece. In the present embodiment, the distal end of the first clip piece 131 in the form of a double-layered elastic piece structure is provided with a traction hole; specifically, traction hole is provided at a position of the inner elastic piece corresponding to the outer elastic piece for allowing a traction wire to penetrate therethrough, so as to force the first clip piece 131 to be opened relative to the first inner clip arm 121 by applying the traction force. Correspondingly, the double-layered elastic piece structure of the second clip piece 132 and the arrangement mode thereof may be the same as the first clip piece 131.

In the present disclosure, the outer clip element 14 is an integrally formed member, the embodiment of which is illustrated in FIG. 2 or FIG. 10. Herein, the outer clip element 14 is a U-shaped element including a first outer clip arm 141, a second outer clip arm 142, a distal end part 143 which is integrally formed of distal ends of the first outer clip arm 141 and the second outer clip arm 142, and hinge parts 123 formed at the proximal ends of the first outer clip arm 141 and the second outer clip arm 142 respectively. The first outer clip arm 141 has a third stiffness, and the second outer clip arm 142 also has the same third stiffness. In the present disclosure, the overall stiffness of the first outer clip arm 141 or the second outer clip arm 142 is defined as the third stiffness, and the stiffness distribution close to the proximal end of the first outer clip arm 141 or the second outer clip arm 142 is smaller than that close to the distal end thereof. A clamping space is formed between the first outer clip arm 141 and the second outer clip arm 142; and the first outer clip arm 141 and the second outer clip arm 142 maintain opposite clamping forces in the case that the spacer element 11, the clip piece assembly 13 and the inner clip assembly 12 are located in the clamping space.

In some embodiments, the outer clip element 14 may also be referred to as a U-like shaped structure formed by two assistant pieces, and the first outer clip arm 141 or the second outer clip arm 142 of the outer clip element 14 may also be referred to as a clamping strip.

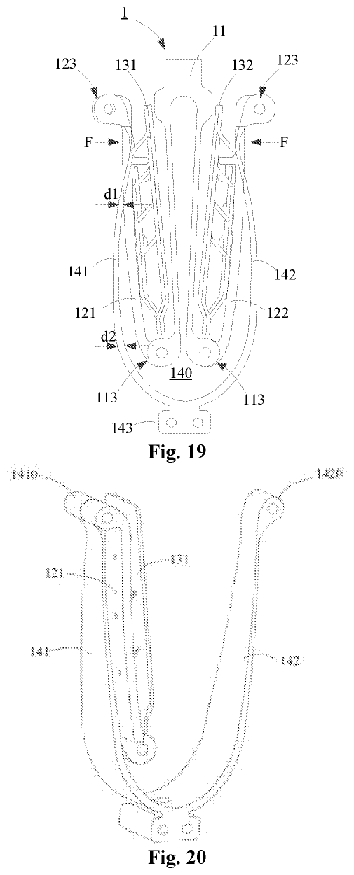

Referring to FIG. 19 in conjunction with FIGS. 2 to 4, in which FIG. 19 is a schematic diagram illustrating a clamping state of the outer clip element in an embodiment of the present disclosure. As illustrated in the figure, the outer clip element 14 includes a distal end part 143, as well as a first outer clip arm 141 and a second outer clip arm 142 which are integrally formed with the distal end part 143 and have a third stiffness respectively; the proximal ends of the first outer clip arm 141 and the second outer clip arm 142 are respectively hinged to the second hinge parts 123. In the present embodiment, the hinge structures formed at the proximal ends of the first outer clip arm 141 and the second outer clip arm 142 are two hinges which are separated from each other and curled outwardly for hinging to the second hinge parts 123 respectively at the distal ends of the first inner clip arm 121 and the second inner clip arm 122 of the inner clip assembly. In this way, in the case that the first inner clip arm 121 and the second inner clip arm 122 of the inner clip assembly are opened towards both sides by means of a downward movement of the spacer element 11 so as to be stretched through a hinged movement of the second hinge parts 123, and in the case that the spacer element 11 continues to move downward to drive the first inner clip arm 121 and the second inner clip arm 122 to generate an inward clamping force, the first outer clip arm 141 and the second outer clip arm 142 can generate clamping forces in opposite directions due to their own elastic restoring force, so that the spacer element 11, the clip piece assembly 13 and the inner clip assembly 12 are positioned in the clamping space, and the first outer clip arm 141 and the second outer clip arm 142 maintain their clamping forces F in opposite directions for each assembly/element in the clamping space due to their own elasticity.

In the embodiment illustrated in FIG. 2 or FIG. 3 and FIG. 19, the width of the proximal end of the first outer clip arm 141 or the second outer clip arm 142 is as same as that of the distal end part 143, that is, both the first outer clip arm 141 and the second outer clip arm 142 are plate bodies with uniform widths. In the present embodiment, in order to ensure that the stiffness distribution close to the proximal end of the first outer clip arm 141 or the second outer clip arm 142 is smaller than that close to the distal end, the first outer clip arm 141 or the second outer clip arm 142 has a structure with thickness gradually increased from the proximal end to the distal end part 143. As illustrated in FIG. 19, the thickness of the plate body close to the proximal end of the first outer clip arm 141 or the second outer clip arm 142 is d1, while the thickness of the plate body close to the distal end part 143 of the first outer clip arm 141 or the second outer clip arm 142 is d2, and d1<d2. In this way, the part of the first outer clip arm 141 or the second outer clip arm 142 close to the proximal end thereof has greater elastic capacity; and correspondingly, the part of the first outer clip arm 141 or the second outer clip arm 142 close to the distal end part 143 thereof has relatively weaker elastic capacity. In the present disclosure, the stiffness distribution of the first outer clip arm 141 or the second outer clip arm 142 including the proximal end and the distal end part 143 is gradually changed instead of suddenly changed.

In the embodiment where the first outer clip arm 141 or the second outer clip arm 142 is a plate body with uniform width, the hinge structures formed at the proximal ends of the first outer clip arm 141 and the second outer clip arm 142 are two hinges 1410 and 1420 which are separated from each other and curled outwardly, so that the hinge structure 1231 or 1232 at the distal end of the first inner clip arm 121 or the second inner clip arm 122 is hinged to the two hinges 1410 and 1420 which are separated from each other and curled outwardly, thereby forming a hinge connection with stable privation and ensuring the stress balance of the hinge point.

In an embodiment, the width of the first outer clip arm 141 or the second outer clip arm 142 is greater than that of the first inner clip arm 121 or the second inner clip arm 122, and the width of the first outer clip arm 141 or the second outer clip arm 142 is greater than that of the first clip piece 131 or the second clip piece 132, so that in the case that the spacer element 11, the inner clip assembly and the clip piece assembly are all folded into the clamping space, they can be shielded by the outer clip element 14.

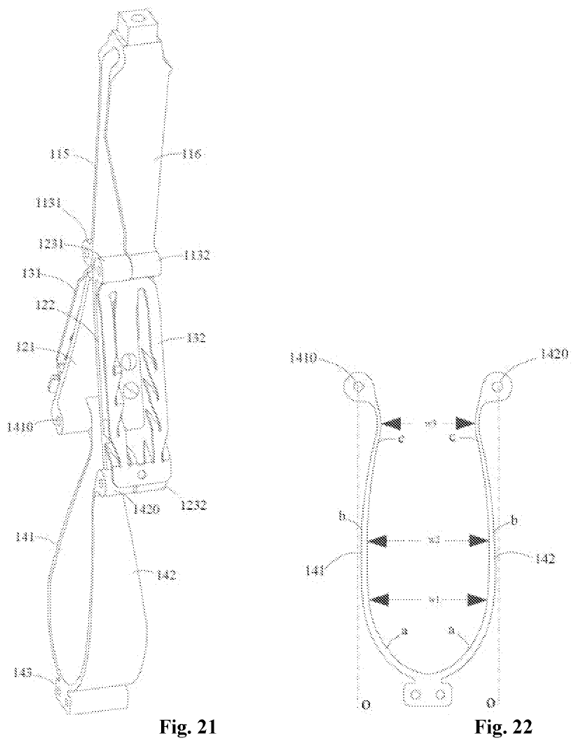

Referring to FIG. 20, which is a schematic diagram illustrating a structure of an outer clip element in an embodiment of the present disclosure. As illustrated in the figure, in the present embodiment, the hinge structures formed at the proximal ends of the first outer clip arm 141 and the second outer clip arm 142 are hinges 1410 and 1420 which are curled outwardly, and the hinges 1410 and 1420 of the first outer clip arm 141 and the second outer clip arm 142 are staggered with each other, so that the proximal ends of the first outer clip arm 141 and the second outer clip arm 142 are respectively hinged to the second hinge parts 123 at the distal ends of the first inner clip arm 121 and the second inner clip arm 122 (for convenience of understanding, the inner clip arm and the clip piece at only one side are illustrated in FIG. 20). The hinged portion connected to the hinge point at the second hinge part 123 has a relatively smaller width and results in less friction force, so that in the case that the driving shaft 20 drives the spacer element 11 to move towards the distal end part 143 of the outer clip element 14 to open the two inner clip arms of the inner clip element relative to each other, which in turn pushes the first outer clip arm 141 and the second outer clip arm 142 to be opened relative to each other, the force exerted by the two inner clip arms would not be affected by the small friction force at the hinged portion. In the structure illustrated in FIG. 12, the width of the proximal end of the first outer clip arm 141 or the second outer clip arm 142 is different from the width of the distal end part 143 thereof, that is, the first outer clip arm 141 and the second outer clip arm 142 are plate bodies with non-uniform widths. Specifically, by taking the first outer clip arm 141 as an example, it is a structure with a width gradually decreased from its distal end part 143 towards its proximal end; and accordingly, the second outer clip arm 142 also has the same structure, as illustrated in FIG. 21, which is a schematic diagram of a valve repair device in another embodiment of the present disclosure.

Referring to FIG. 22, which illustrates a side view of an outer clip element in an embodiment of the present disclosure. As illustrated in the figure, in the present embodiment, in order to reduce the force required for pushing the first outer clip arm 141 and the second outer clip arm 142 to be opened relative to each other, a projection point O of the hinge 1410 or 1420 at the proximal end of the first outer clip arm 141 or the second outer clip arm 142 is fallen outside the first outer clip arm 141 or the second outer clip arm 142 (as illustrated by the dashed line in FIG. 22). In the present embodiment, by taking the first outer clip arm 141 as an example, the hinge structure at the proximal end of the first outer clip arm 141 is a hinge 1410 which is curled outwardly, with an axis center of curling located outside the projection of the first outer clip arm 141, so that the second hinge part 123 has a better force imposing point. Correspondingly, the hinge structure 1420 at the proximal end of the second outer clip arm 142 also has the same structural configuration. In the present embodiment, less force is required for the first inner clip arm 121 and the second inner clip arm 122 to push the first outer clip arm 141 and the second outer clip arm 142 to be opened relative to each other.

In the embodiment illustrated in FIG. 22, each of the first outer clip arm 141 and the second outer clip arm 142 extends from the distal end part 143 towards the proximal end thereof, and includes a first curved section a, a second curved section b, and a third curved section c; herein a maximum spacing distance w1 between the first curved sections a of the first and second outer clip arm 141 and 142 is smaller than a maximum spacing distance w2 between the second curved sections b of the first and second outer clip arm 141 and 142; and the maximum spacing distance w1 between the first curved sections a of the first and second outer clip arm 141 and 142 is greater than or equal to a minimum spacing distance w3 between the third curved sections c of the first and second outer clip arm 141 and 142; that is, w3≤w1<w2.

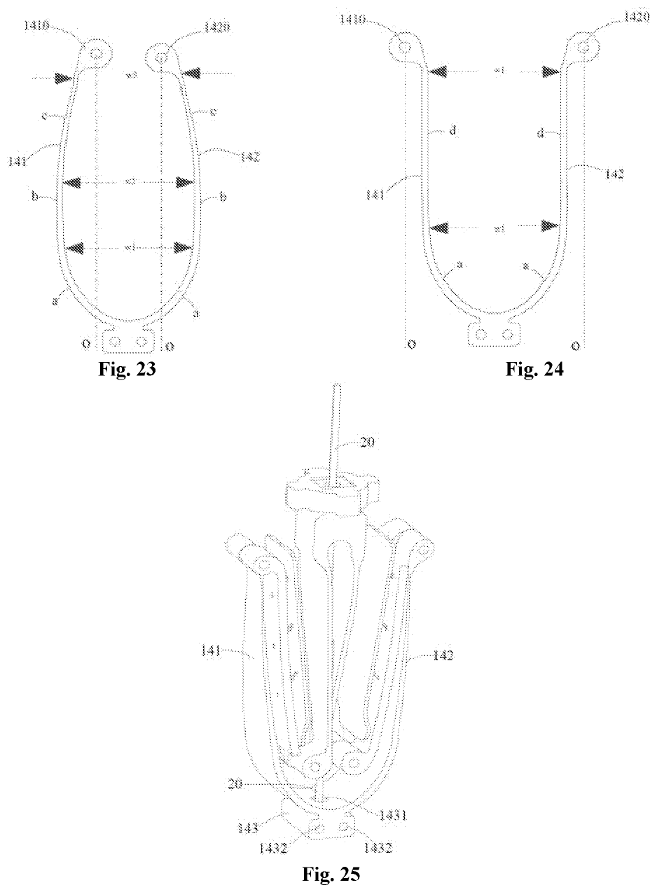

Referring to FIG. 23, which illustrates a side view of an outer clip element in another embodiment of the present disclosure. As illustrated in the figure, in the present embodiment, the outer clip element 14 is a U-shaped component; each of the first outer clip arm 141 and the second outer clip arm 142 extends from the distal end part 143 towards the proximal end thereof, and includes a first curved section a, a second curved section b and a third curved section c. In the present embodiment, the maximum spacing distance between the first curved sections a of the first and second outer clip arms 141 and 142 is w1, the maximum spacing distance between the second curved sections b of the first and second outer clip arms 141 and 142 is w2, and the minimum spacing distance between the third curved sections c of the first and second outer clip arms 141 and 142 is w3, which satisfy w3≤w1<w2. FIG. 22 and FIG. 23 illustrate two structures for implementing the above-described relationships among spacing distances, respectively; that is, the implementation in which the projection point O of the hinge at the proximal end of the first outer clip arm 141 or the second outer clip arm 142 is fallen outside the first outer clip arm 141 or the second outer clip arm 142, and the implementation in which the projection point O of the hinge at the proximal end of the first outer clip arm 141 or the second outer clip arm 142 is fallen inside the first outer clip arm 141 or the second outer clip arm 142.

In the embodiment illustrated in FIG. 23, in order to increase the opposite clamping forces of the first outer clip arm 141 and the second outer clip arm 142 under normal conditions, the clamping space delimited by the first outer clip arm 141 and the second outer clip arm 142 has a smaller opening, that is, the projection point O of the hinge at the proximal end of the first outer clip arm 141 or the second outer clip arm 142 is fallen inside the first outer clip arm 141 or the second outer clip arm 142 and is further fallen within the clamping space. In the present embodiment, a greater force is required for the first inner clip arm 121 and the second inner clip arm 122 to push the first outer clip arm 141 and the second outer clip arm 142 to move towards an opposite direction.

Referring to FIG. 24, which illustrates a side view of an outer clip element in yet another embodiment of the present disclosure. As illustrated in the figure, in the present embodiment, each of the first outer clip arm 141 and the second outer clip arm 142 extends from the distal end part 143 towards the proximal end thereof, and includes a curved section a and a straight section d. In the present embodiment, a maximum spacing distance between the curved sections a of the first and second outer clip arms 141 and 142 is w1, and a maximum spacing distance between the straight sections d of the first and second outer clip arms 141 and 142 is also w1, as illustrated in FIG. 24.

Referring to FIG. 25, which is a schematic diagram illustrating a cooperation between an outer clip element and a driving shaft in an embodiment of the present disclosure. As illustrated in the figure, the first outer clip arm 141 and the second outer clip arm 142 of the outer clip element 14 are integrally formed with the distal end part 143, and the distal end part 143 has a screw hole 1431 for threaded connection with the driving shaft 20 of the delivery device. Under the initial configuration of the valve repair device, the driving shaft 20 of the delivery device passes through the through hole of the connecting part of the spacer element 11, and through the gap between the first spacer plate 115 and the second spacer plate 116 to be screwed into the screw hole 1431 of the distal end part 143. In the case that the driving shaft 20 withdraws towards the proximal end, the spacer element 11 is driven to move linearly relative to the distal end part 143 and then drives the first and second inner clip arms 121 and 122 hinged to the first hinge parts 113 to be opened relative to each other, which in turn pushes the first and second outer clip arms 141 and 142 hinged to the second hinge parts 123 to be opened relative to each other. In the case that the first and second outer clip arms 141 and 142 with smaller stiffness are pushed to be opened relative to each other to the maximum extent, the distal ends of the spacer element 11 as well as the proximal ends of the first and second inner clip arms 121 and 122 that are connected with the distal ends of the spacer element 11 through the first hinge parts 113 are approaching the distal end part 143 of the outer clip element. At this time, the first and second inner clip arms 121 and 122 are folded to move the proximal ends thereof to be close to the distal end part 143 of the outer clip element 14, and the first and second outer clip arms 141 and 142 force the first and second inner clip arms 123 on both sides of the inner clip assembly to be attached onto two sides of the spacer element 11 due to the elastic restoring force. At this time, the valve repair device is in the closed state as illustrated in FIG. 25. After this operation, the driving shaft 20 of the delivery device is disengaged from the screw hole 1431 by rotation, so that the driving shaft 20 is pulled away from the valve repair device.

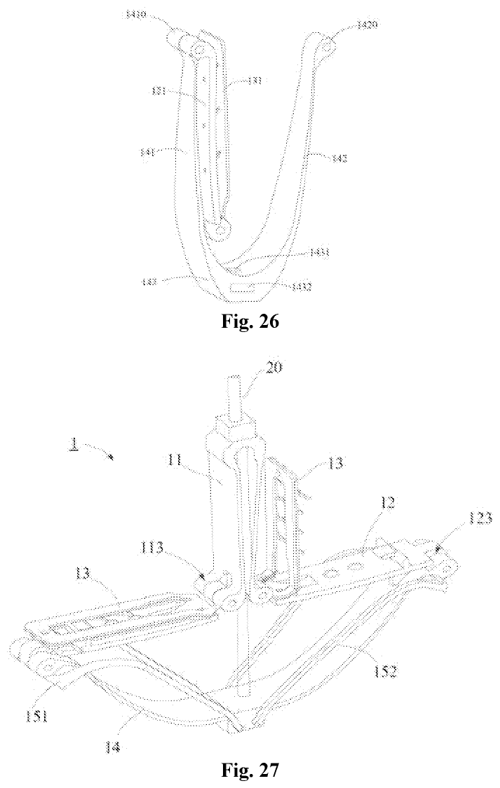

Referring to FIG. 26, which is a schematic diagram illustrating a structure of an outer clip element in further another embodiment of the present disclosure. As illustrated in the figure, in the present embodiment, the distal end part 143 of the outer clip element 14 is an arc-shaped thickening structure, or is a base structure integrally formed at the bottom of the outer clip element 14.

In an embodiment, two sides of the distal end part 143 of the outer clip element 14 are provided with a pivotal hole 1432 respectively. In the present embodiment, the two sides of the distal end part 143 of the outer clip element 14 are respectively two side surfaces in the second direction, and two pivotal holes 1432 are provided on each of the two side surfaces for pivotal connection with an outer frame assembly, so that two outer frames of the outer frame assembly can be rotatably pivotally connected to both sides of the distal end part 143 of the outer clip element 14.

In some embodiments, the pivotal holes 1432 respectively arranged on both sides of the distal end part 143 of the outer clip element 14 may also be referred to as side-surface slots.

Referring to FIG. 27, which is a schematic diagram illustrating a configuration of an outer frame assembly in a valve repair device in an embodiment of the present disclosure. As illustrated in the figure, in the present embodiment, the valve repair device 1 further includes an outer frame assembly 15 wrapped by a covering material, and the outer frame assembly 15 includes a first outer frame 151 and a second outer frame 152, both of which are frame structures formed by bending strip-shaped pieces. The first outer frame 151 and the second outer frame 152 are rotatably pivoted at the pivotal holes 1432 in both sides of the distal end part 143 of the outer clip element 14, so as to cover the first outer clip arm 141 and the second outer clip arm 142 respectively in the folding state thereof.

In some embodiments, the first outer frame 151 or the second outer frame 152 of the outer frame assembly may also be referred to as an auxiliary clamping piece.

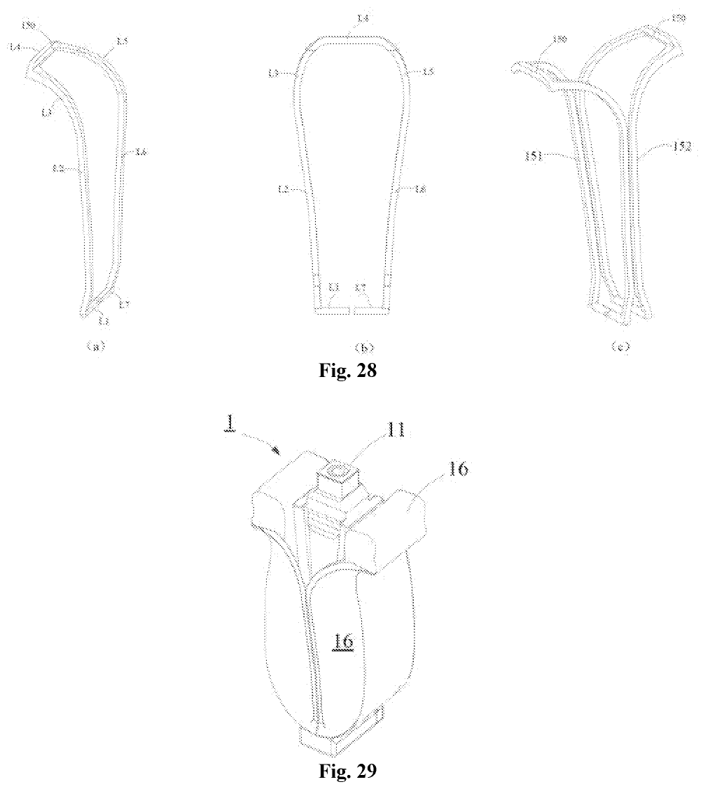

Referring to FIG. 28, which is a schematic diagram illustrating a structure of an outer frame of an outer frame assembly in an embodiment of the present disclosure. For the convenience of understanding, FIG. 28 provides views of three states, in which (a) of FIG. 28 is a perspective view of the structure of the outer frame, (b) of FIG. 28 is a front view o of the structure of the outer frame, and (c) of FIG. 28 is a schematic view of the first and second outer frames in a closed state. As illustrated in the figures, in the present embodiment, the first outer frame 151 or the second outer frame 152 is bent into a valve leaflet structure, which should be understood in such a way that the bending structure of the first outer frame 151 or the second outer frame 152 wrapped by a covering presents a structure similar to a petal or a plant leaf.

As illustrated in FIG. 28, by taking the first outer frame 151 as an example, the first outer frame 151 made of a strip-shaped piece includes a first section L1 extending by a first length in the second direction, a second section L2 extending by a second length in the third direction, a third section L3 in an outwardly bending shape which is bent by a third length, a fourth section L4 extending by a fourth length in the second direction, a fifth section L5 in an outwardly bending shape which is bent by the third length, a sixth section L6 extending by the second length in the third direction, and a seventh section L7 extending by the first length in the second direction; herein the length of the first section L1 is equal to that of the seventh section L7, the length of the second section L2 is equal to that of the sixth section L6, the length of the third section L3 is equal to that of the fifth section L5; the first section L1 and the seventh section L7 are pivot parts which are pivoted at the pivotal holes in both sides of the distal end part 143 of the outer clip element 14; and the second section L2, the third section L3, the fourth section L4, the fifth section L5 and the sixth section L6 constitute a frame structure which extends from the pivot parts towards the proximal end and has a gradually increased width; the third section L3 and the fifth section L5 are outwardly bending structures formed at the proximal end of the frame structure.

In an embodiment, both the first outer frame 151 and the second outer frame 152 of the outer frame assembly are wrapped by a covering material. In order to allow the covering material to be wrapped on the outer frames more firmly, a folding-edge structure 150 is provided at the proximal end of the frame structure, and the folding-edge structure 150 is formed on the fourth section L4 extending by the fourth length in the second direction described above, so as to be engaged with the covering material in a better way.

In an embodiment, in the case that the first outer frame 151 and the second outer frame 152 of the outer frame assembly cover the first outer clip arm 141 and the second outer clip arm 142 in a folding state, the width of the gap between the middle portion of the first outer frame 151 and the middle portion of the second outer frame 152 is not greater than 3 mm; preferably, the width of the gap between the middle portion of the first outer frame 151 and the middle portion of the second outer frame 152 is 1 mm-3 mm.

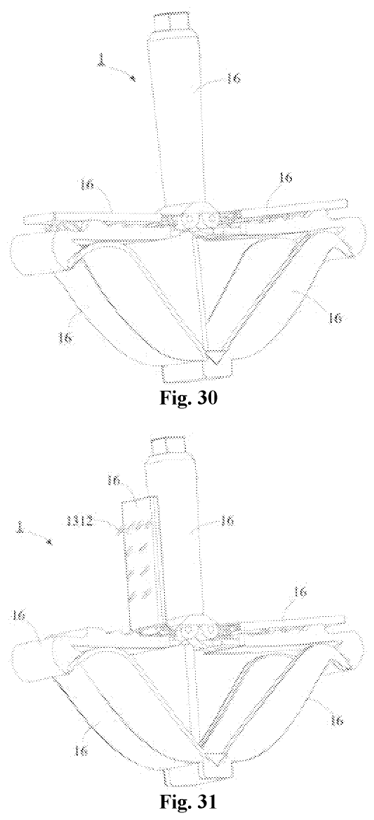

Referring to FIG. 29 and FIG. 30, in which FIG. 29 is a schematic diagram illustrating a closed state of a valve repair device wrapped by a covering material in an embodiment of the present disclosure, and FIG. 30 is a schematic diagram illustrating an opened state of a valve repair device wrapped by a covering material in an embodiment of the present disclosure. As illustrated in the figures, in the present embodiment, the valve repair device 1 further includes a covering material 16 for wrapping the spacer element 11, the inner clip assembly 12, the clip piece assembly 13, the outer clip element 14, and the outer frame assembly 15. In an embodiment, the covering material 16 may be configured to avoid or reduce blood flowing through the valve repair device and/or to promote the valve repair device endothelialization. In the embodiment, in the case that the valve repair device presents a closed state after capturing and clamping the natural valve leaflets, most of the natural valve leaflets being clamped would be wrapped by the covering material 16, thereby reducing the possibility of thrombosis and shortening the time required for endothelialization.

In some embodiments, the covering material 16 may be cloth or woven material, such as PET, velvet, or other suitable woven materials. In some other embodiments, instead of or in addition to woven material or woven fabric, the covering material 16 may include a coating applied on the valve repair device, such as a polymer (e.g., natural polymer) or a high molecular material. In an embodiment, the covering material 16 is coated with or carried with functional drugs for accelerating the endothelialization of the valve repair device. In an embodiment, the cloth is, for example, polyethylene cloth, such as fine mesh polyethylene cloth; the cloth covering can seal the blood on the surface of the spacer element and/or promote rapid the valve repair device endothelialization.

Referring to FIG. 31, which is a schematic diagram illustrating an opened state of a valve repair device wrapped by a covering material in an embodiment of the present disclosure. As illustrated in the figure, in the present embodiment, the covering material 16 wrapped on the spacer element 11 and the clip piece assembly 13 is an integrally woven material; the covering material wrapped on the inner clip assembly 12, the outer clip element 14 and the outer frame assembly 15 is integrally woven material. The hangnail-shaped protrusions on the clip piece assembly 13 are exposed by penetrating through the covering material 16 so as to capture the valve. In other words, the valve repair device includes two independent woven fabrics, herein, the spacer element 11 as well as the first and second clip pieces 131 and 132 of the clip piece assembly 13 are wrapped by one piece of woven fabric, while the first and second inner clip arms 121 and 122 of the inner clip assembly 12, the first and second outer clip arms 141 and 142 and the distal end part 143 of the outer clip element 14, as well as the first and second outer frames 151 and 152 of the outer frame assembly 15 are wrapped by another piece of woven fabric, so that each component of the valve repair device possesses improved tensile resistance and fatigue resistance.

In the present disclosure, the term “integrally woven” means that part of the weave threads of the woven fabric is continuous and uninterrupted. For example, the woven material wrapped on the inner clip assembly 12, the outer clip element 14 and the outer frame assembly 15 is integrally woven, which means that part of the weave threads wrapped on the inner clip assembly 12, the outer clip element 14 and the outer frame assembly 15 is continuous and uninterrupted. That is, the inner clip assembly 12, the outer clip element 14, and the outer frame assembly 15 are wrapped by an integral piece of woven fabric, so that the woven fabric can not only improve the sealing effect of covering the components wrapped therein but also provide the components wrapped therein with improved tensile resistance and fatigue resistance.

In an embodiment, in the state that the first outer frame 151 and the second outer frame 152 of the outer frame assembly 15 are wrapped by the covering material 16, the outer frame assembly 15 presents a valve leaflet-like structure, and a wrapped space presented by the valve leaflet-like structure can wrap the first outer clip arm 141 or the second outer clip arm 142 therein, thereby shielding the first outer clip arm 141 or the second outer clip arm 142. In the present embodiment, the first outer frame 151 and the first outer clip arm 141 are wrapped together by the covering material 16 so as to be integrated into a whole, while the second outer frame 152 and the second outer clip arm 142 are wrapped together by the covering material 16 so as to be integrated into a whole.

In the embodiment, in the state that the first inner clip arm 121 and the second inner clip arm 122 of the inner clip assembly 12 are wrapped by the covering material 16, the tabs of the first clip piece 131 and the second clip piece 132 respectively fixed on the first inner clip arm 121 and the second inner clip arm 122 are also wrapped together, so that the first inner clip arm 121 and the tab of the first clip piece 131 are wrapped together and integrated into a whole, while the second inner clip arm 122 and the tab of the second clip piece 132 are wrapped together and integrated into a whole.

In the embodiment, in the state that the spacer element 11 is wrapped by the covering material 16, the ear-shaped block on the connecting part of the spacer element 11 for snap-fitted connection with the release structure of the delivery device is exposed out of the covering material 16, so as to be snap-fitted with the release structure. In the present embodiment, the first spacer plate 115 and the second spacer plate 116 of the spacer element 11, as a whole, are wrapped by the covering material 16, and the gap therebetween is covered by the covering material 16 and shielded therein.

In another embodiment, the valve repair device 1 further includes a covering material 16 for wrapping the spacer element 11, the inner clip assembly 12, the clip piece assembly 13, and the outer clip element 14, herein, hangnail-shaped protrusions on the clip piece assembly 13 are exposed by penetrating through the covering material 16 so as to capture the valve. In the present embodiment, the covering material 16 wrapped on the spacer element 11 and the clip piece assembly 13 is an integrally woven material; and the woven material wrapped on the inner clip assembly 12 and the outer clip element 14 is integrally woven.

In order to further describe the inventive concept and principle of the present disclosure, referring to FIGS. 2 to 4; as illustrated in the figures, the valve repair device 1 may be transited between multiple states in the actual surgical application, such as a stretch state, an opened state and a closed state. The valve repair device 1 is restricted to a stretch state when it is arranged inside the delivery catheter/delivery sheath of the delivery device. The delivery sheath of the delivery device is inserted into the left atrium through the atrial septum, and the valve repair device 1 is protruded out of the delivery sheath to present a stretch state thereof. At this time, the connecting part at the proximal end of the spacer element 11 is in snap-fitted connection with the distal end of the delivery catheter/delivery sheath, the first inner clip arm 121 and the second inner clip arm 122 that are respectively hinged to the first hinge parts 113 at the distal end of the spacer element 11 are almost in contact with each other, and the first outer clip arm 141 and the second outer clip arm 142 of the outer clip element 14 that are respectively hinged to the second hinge parts 123 at the distal ends of the first inner clip arm 121 and the second inner clip arm 122 present a normal U-shaped configuration, because of the restriction caused by accommodated in the inner space of the delivery catheter/delivery sheath has been removed.

The valve repair device 1 is partially delivered through the mitral valve, and the portion thereof access to the left ventricle is partially opened to present an inverted umbrella shape; in this way, when the natural valve leaflet is to be captured by operating the clip pieces on the inner clip arms at both sides of the valve repair device 1, the first clip piece 131 or the second clip piece 132 is opened relative to the first inner clip arm 121 or the second inner clip arm 122 by manipulating the traction wire passing through the distal end of the first clip piece 131 or the second clip piece 132. At this time, by operating the driving shaft 20 (the central rod) of the delivery device, the spacer element 11 moves towards the distal end part 143 of the outer clip element 14 (which can also be understood that, the distal end part 143 of the outer clip element 14 moves towards the spacer element 11). Due to the rotating axis or pivoting structure provided by the first hinge parts 113, the first inner clip arm 121 and the second inner clip arm 122 respectively hinged to the first hinge parts 113 are simultaneously opened towards both sides (towards the outside); correspondingly, the first and second clip pieces 131 and 132 respectively fixed on the first and second inner clip arms 121 and 122 are also simultaneously opened towards both sides (towards the outside) along with the first and second inner clip arms 121 and 122. Due to the rotation axis or privation provided by the second hinge parts 123 and due to the stiffness (third stiffness) of the first and second outer clip arms 141 and 142 having a U-shaped structure, the first outer clip arm 141 and the second outer clip arm 142 are gradually opened relative to each other to the greatest extent. At this time, the valve repair device 1 presents an opened state like an umbrella.

By operating the driving shaft 20 (the central rod) of the delivery device, the spacer element 11 continues to move towards the distal end part 143 of the outer clip element 14, and the distal end of the spacer element 11 enters the clamping space formed by the first outer clip arm 141 and the second outer clip arm 142 having the U-shaped configuration and gradually approaches the distal end part 143 of the outer clip element 14. At this time, the proximal end and the distal end of each of the first inner clip arm 121 and of the second inner clip arm 122 that are respectively hinged to the first hinge part 113 and second hinge part 123 exchange their positions. During this process, the first clip piece 131 and the second clip piece 132 are released by controlling the traction wire, so that one side of the natural valve is clamped between the first clip piece 131 and the first inner clip arm 121, and the other side of the natural valve is clamped between the second clip piece 132 and the second inner clip arm 122; the spacer element 11 continues to move downwards to drive the first inner clip arm 121 and the second inner clip arm 122 to be fold inwardly (in the direction of the central axis of the spacer element 11), due to the inversion of the first inner clip arm 121 and the second inner clip arm 122, the supporting forces applied on the first outer clip arm 141 and the second outer clip arm 142 would be reduced, and the first outer clip arm 141 and the second outer clip arm 142 will clamp in opposite directions by their own elastic restoring force, so that the spacer element 11, the clip piece assembly 13, and the inner clip assembly 12 are positioned in the clamping space; furthermore, the first outer clip arm 141 and the second outer clip arm 142 maintain the opposite clamping forces on the assemblies/elements in the clamping space due to their own elasticity, so that the valve repair device 1 is in a closed state.

At this time, by operating the delivery device, the driving shaft 20 is disengaged from the screw hole 1431 of the outer clip element 14, then, releasing the snap-fitted connection with the ear-shaped blocks on the spacer element 11 by operating the release structure at the distal end of the delivery catheter/delivery sheath of the delivery device, so as to pull the driving shaft 20 away; and then pulling the traction wire away from the traction holes in the first clip piece 131 and the second clip piece 132, in this way, the valve repair device 1 is kept on the natural valve leaflets, thereby completing the procedure of partly closing the natural valve leaflets.

The valve repair device provided by the present disclosure adopts two hinge structures to hinge the spacer element with the inner clip assembly at a first position, and to hinge the inner clip assembly with the outer clip element having a U-shaped structure at a second position, respectively. Compared with the related art (for example, the valve repair clamp disclosed in China Patent Publication No. CN111449805A) in which only one hinge structure is adopted and the other position is provided with a folding structure, the two hinge structures adopted in the present disclosure achieve better flexibility. In the present disclosure, it requires for less force in the case that the driving shaft drives the spacer element to move towards the distal end part of the outer clip element, which is beneficial to the precision control of surgeons during operation. Furthermore, in the present disclosure, an integrally formed U-shaped outer clip element is adopted, and the stiffness design of the outer clip arms thereof is utilized to continuously maintain opposite clamping forces for each assembly/element in the clamping space, which achieves more stable performance as compared with the related art utilizing mechanical locking/attaching mode to ensure the clamping effect. Moreover, the integrally formed U-shaped outer clip element adopted in the present disclosure provides a simpler structure and a simpler clamping mode with more stable clamping effect, as compared with the related art which provides an elastic clamping force by frames/strip-shaped pieces. Additionally, as compared with the outer clip arms and inner clip arms formed by folding metallic woven materials in the related art (for example, the woven mesh-like valve repair fixture disclosed in China Patent Publication No. CN112402061A), the present disclosure provides a simpler manufacturing process.

The second aspect of the present disclosure also provides a fixing mechanism for a clip piece assembly. In an embodiment, the fixing mechanism for the clip piece assembly is used for fixing the clip piece on the valve repair device described in the above embodiments. Specifically, the clip piece assembly includes a first clip piece and a second clip piece, the stiffness of the first clip piece or the second clip piece of the clip piece assembly is smaller than that of the clip plate. In an embodiment, each of the first clip piece and the second clip piece is an elastic piece structure which is integrally formed. In an embodiment, the first clip piece or the second clip piece is made from a piece of shape memory alloy sheet, for example, by cutting Nitinol with laser.

The fixing mechanism includes a clip plate and a clip piece fixed on the clip plate. For the convenience of explanation, in the embodiments described with reference to the following drawings, the clip piece is, for example, any one of the first clip piece and the second clip piece described in the above embodiments; and the clip plate is, for example, any one of the first inner clip arm and the second inner clip arm described in the above embodiments. Therefore, in the following embodiments, both the first inner clip arm and the second inner clip arm refer to the clip plate in the fixing mechanism; both the first clip piece and the second clip piece refer to the clip piece in the fixing mechanism, which is specially noted herein.

Referring to FIG. 10, the proximal end of the clip plate of the first inner clip arm 121 or the second inner clip arm 122 is hinged to the first hinge part 113 of the spacer element 11, and the distal end of the clip plate of the first inner clip arm 121 or the second inner clip arm 122 is provided with the second hinge part 123. In an embodiment, the clip plate of the first inner clip arm 121 or the second inner clip arm 122 has a second stiffness. The second stiffness is smaller than or equal to the first stiffness of the spacer element 11 in an example and greater than the first stiffness of the spacer element 11 in another example. In the present disclosure, the second stiffness refers to an acting force which is applied on the proximal and distal ends of the first inner clip arm 121 or the second inner clip arm 122 by the spacer element 11 and the outer clip element 14, and is not sufficient to deform the plate body of the first inner clip arm 121 or the second inner clip arm 122.

In an embodiment, the spacer element 11, the inner clip assembly 12 and the outer clip element 14 can be made of the same material, for example, the spacer element 11, the inner clip assembly 12 and the outer clip element 14 can be obtained by cutting Nitinol material or Nickel-Titanium material with laser. In the present embodiment, the plate thickness of the first inner clip arm 121 or the second inner clip arm 122 is greater than that of the first spacer plate 115 or the second spacer plate 116 of the spacer element 11, and the plate thickness of the first inner clip arm 121 or the second inner clip arm 122 is greater than that of the first outer clip arm or the second outer clip arm of the outer clip element 14, in order to guarantee the stiffness distribution of the spacer element 11, the inner clip assembly 12 and the outer clip element 14.

The clip plate includes a plate body with a preset strength (the strength is determined by the above-mentioned second stiffness), an opening formed in the proximal end of the plate body, and a limiting block formed on the distal end of the plate body; referring to FIG. 11, as illustrated in the present embodiment, the proximal end of the first inner clip arm 121 has a hinge 1231 curled outwardly, and the distal end of the first inner clip arm 121 has an hinge 1232 curled inwardly; correspondingly, the proximal end of the second inner clip arm 122 has a hinge 1231 curled outwardly, and the distal end of the second inner clip arm 122 has a hinge 1232 curled inwardly. Because the hinge points of the first inner clip arm 121 and the second inner clip arm 122 are distributed on different sides of the two ends of the respective plate bodies, in the case that the first inner clip arm 121 and the second inner clip arm 122 are subjected to a pushing force generated by a linear movement of the spacer element 11, they can perform a force transmission more efficiently, so that the driving shaft of the delivery device requires for less pushing force and hence that the two inner clip arms of the inner clip assembly are easier to push the two outer clip arms of the outer clip element 14 to be opened relative to each other.

In the present disclosure, in the embodiment illustrated in FIGS. 2 to 4 and the above-mentioned FIG. 12, a first clip piece 131 and a second clip piece 132 are respectively arranged on the first inner clip arm 121 and the second inner clip arm 122 of the inner clip assembly 12. In the embodiment illustrated in FIG. 11, the plate body close to the proximal end of the first inner clip arm 121 is provided with an opening 1211 for the first clip piece 131 to pass therethrough; specifically, the opening 1211 is configured such that a part of the first clip piece 131 penetrates through one side surface (such as the front surface) of the plate body of the first inner clip arm 121 to be engaged with the other side surface (such as the rear surface). Correspondingly, the plate body close to the proximal end of the second inner clip arm 122 is provided with an opening for the second clip piece 132 to penetrate therethrough; specifically, the opening is configured such that a part of the second clip piece 132 penetrates through one side surface (such as the front surface) of the plate body of the second inner clip arm 122 to be engaged with the other side surface (such as the rear surface). In the present embodiment, the plate body of the first inner clip arm 121 or the second inner clip arm 122 is provided with a fixing hole 1213 for welding or riveting the first clip piece 131 or the second clip piece 132. In the present embodiment, the first clip piece 131 and the second clip piece 132 are fixed on the first and second inner clip arms 121 and 122 respectively by welding, without limiting the present disclosure thereto. In some other embodiments, the first clip piece 131 and the second clip piece 132 can also be fixed on the first and second inner clip arms 121 and 122 respectively by riveting or screwing.

In the embodiment illustrated in FIG. 11, the plate bodies of the first inner clip arm 121 and the second inner clip arm 122 are respectively provided with a limiting block 1212, the limiting blocks 1212 are located close to the distal ends of the plate bodies of the first inner clip arm 121 and the second inner clip arm 122 and have a certain height. The first clip piece 131 and the second clip piece 132 are respectively arranged on the first inner clip arm 121 and the second clip piece 132 in an openable and closable manner. The cooperation between the first clip piece 131 and the first inner clip arm 121 would be described below by way of example: in the case where the first clip piece 131 is closed towards the first inner clip arm 121 to approach the surface (for example, the front surface) of the first inner clip arm 121, because the side of the first clip piece 131 facing towards the surface of the first inner clip arm 121 is provided with hangnail-shaped protrusions/puncture needles, a certain gap needs to be preset between the first clip piece 131 and the first inner clip arm 121 in order to ensure that the first clip piece 131 would not be directly attached onto the surface of the first inner clip arm 121 to compress and deform the hangnail-shaped protrusions/puncture needles; as a result, the height of the limiting block determines the relative distance between the first inner clip arm 121 and the first clip piece 131. Accordingly, the second clip piece 132 and the second inner clip arm 122 are cooperated with each other in the same way.

In an embodiment, the certain gap preset between the first clip piece 131 and the first inner clip arm 121 is 1 mm-3 mm. Specifically, in some embodiments, the gap is 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm or 3 mm.

Moreover, because the first clip piece 131 and the second clip piece 132 are respectively fixed on the plate bodies of the first inner clip arm 121 and the second inner clip arm 122 by welding or riveting, it is necessary to guarantee the positioning accuracy of the fixed positions when fixing the first clip piece and the second clip piece on the plate bodies of the first inner clip arm 121 and the second inner clip arm 122 respectively as described above. The limiting blocks 1212 on the plate bodies of the first and second inner clip arms 121 and 122 play the role of positioning here, and the case that the first clip piece 131 is fixed on the first inner clip arm 121 is described by way of example: a part of the first clip piece 131 (for example, an elastic structure in the form of a tab on the first clip piece 131 would be illustrated later) penetrates through the opening in the plate body of the first inner clip arm 121 close to the proximal end thereof and reaches the other side surface, then the first clip piece 131 is fixed there by welding or riveting, and both the hangnail-shaped protrusion/puncture needle on the first clip piece 131 and the limiting block 1212 are located in a positioning space between the first clip piece 131 and the first inner clip arm 121; in this way, whether the first clip piece 131 is positioned accurately or not can be judged by the relative position of the hangnail-shaped protrusion/puncture needle on the first clip piece 131 and the limiting block 1212, so as to ensure that the first clip piece 131 can be accurately fixed at an ideal position on the first inner clip arm 121. In actual operation, the hangnail-shaped protrusion/puncture needle on the first clip piece 131 can be abutted against a root portion of the limiting block 1212, so as to position the first clip piece 131 at the ideal position on the first inner clip arm 121.

Referring to FIG. 13, which is a schematic diagram illustrating a cooperation between a puncture needle on the clip piece and a puncture hole in an inner clip arm in an embodiment of the present disclosure. As illustrated in the figure, in the embodiment, the plate body of the first inner clip arm 121 or the second inner clip arm 122 is provided with a puncture hole or a puncture groove 1214 corresponding to the hangnail-shaped protrusion/puncture needle 1312 on the first clip piece 131 or the second clip piece 132, so that in the case that the first clip piece 131 and the second clip piece 132 are respectively closed with respect to the first inner clip arm 121 and the second inner clip arm 122, the hangnail-shaped protrusions/puncture needles on the first clip piece 131 or the second clip piece 132 can penetrate into the puncture holes or the puncture grooves. Still referring to the case where the first clip piece 131 is closed with respect to the first inner clip arm 121 by way of example, in the case that the natural valve leaflets are captured between the first clip piece 131 and the first inner clip arm 121, the hangnail-shaped protrusions/puncture needles on the first clip piece 131 penetrate through the natural valve leaflets and then penetrate into the puncture holes or puncture grooves in the plate body of the first inner clip arm 121, so as to hold the natural valve leaflets between the first clip piece 131 and the first inner clip arm 121 more stably, and to avoid the risk that the valve repair device would fall off along with heart beating.

The clip piece of the first clip piece or the second clip piece is fixed on the clip plate (the first inner clip arm or the second inner clip arm), and includes an elastic piece, a tab extending from the proximal end of the elastic piece to penetrate through the opening to be fixed on the rear surface of the plate body, and hangnail-shaped protrusions which are formed on the elastic piece and face towards the front surface of the piece body; the distal end of the elastic piece is provided with a traction part.

Referring to FIG. 10 again, as illustrated in the figure, the clip piece assembly 13 includes a first clip piece 131 and a second clip piece 132. The first clip piece 131 of the clip piece assembly has a fourth stiffness, the second clip piece 132 also has a fourth stiffness, and the fourth stiffness is smaller than the third stiffness, that is, the first clip piece 131 and the second clip piece 132 are more flexible and elastic as compared to the first outer clip arm 141 and the second outer clip arm 142 of the outer clip element 14. In the present disclosure, each of the first clip piece 131 and the second clip piece 132 is an integrally formed elastic piece structure. In the present embodiment of the present disclosure, each of the first clip piece 131 and the second clip piece 132 is made from a sheet material of shape memory alloy, for example, by cutting Nitinol with laser.

In some embodiments, the clip piece assembly 13 is also referred to as a clamping structure. Correspondingly, the first clip piece 131 can also be referred to as a first clamping piece, and the second clip piece 132 can also be referred to as a second clamping piece.

Referring to FIG. 14, which is a schematic diagram illustrating a stretch state and a closed state of a clip piece assembly in an embodiment of the present disclosure. As illustrated in the figure, in the present embodiment, the first clip piece 131 is arranged on the first inner clip arm 121, and the distal end of the first clip piece 131 is provided with a traction part 1313; specifically, the traction part 1313 is a traction hole 1313 for a traction wire to penetrate therethrough. In an embodiment, the traction hole 1313 is formed in the piece body of the first clip piece 131; in another embodiment, the traction hole 1313 can also be a perforated structure formed at the distal end of the first clip piece 131 and curled outwardly. The traction hole 1313 of the first clip piece 131 is moved away relative to the first inner clip arm 121 (in the state of the first clip piece as illustrated in FIG. 14) when applied with a traction force by the traction wire 30; and correspondingly, while the traction force is removed, the first clip piece 131 is closed relative to the first inner clip arm 121 (the state of the second clip piece as illustrated in FIG. 14 is the state where the traction force of the traction wire is removed) due to its own elastic restoring force (in the state of the second clip piece where the traction force is removed, as illustrated in FIG. 14). Correspondingly, the second clip piece 132 is arranged on the second inner clip arm 122, and the distal end of the second clip piece 132 is provided with a traction part 1323; specifically, the traction part 1323 is a traction hole for the traction wire 30 to pass therethrough. In an embodiment, the traction hole is formed in the piece body of the second clip piece 132; in another embodiment, the traction hole is a perforated structure formed at the distal end of the second clip piece 132 and curled outwardly. The traction hole of the second clip piece 132 is moved away relative to the second inner clip arm 122 when applied with a traction force by the traction wire 30; and correspondingly, while the traction force is removed, the second clip piece 132 is closed relative to the second inner clip arm 122 due to its own elastic restoring force. In the present embodiment, the opened angle of the first clip piece 131 or the second clip piece 132 relative to the first inner clip arm 121 or the second inner clip arm 122 is controlled by the traction force of the traction wire.

In some embodiments, the traction wire 30 is also referred to as a control thread. In an embodiment, the traction wire 30 (control thread) can also be connected with a capture ring 31, and the capture ring 31 is respectively connected with the traction wires in the first clip piece 131 and in the second clip piece 132. The traction wires 30 respectively connected with the first clip piece 131 and the second clip piece 132 are both connected to the same capture ring 31, which is used for recycling the valve repair device 1 of the present disclosure. For example, during the subsequent routine inspection, if it is found that the valve repair device 1 fails to clamp properly and needs to clamp again or needs to be withdrawn for other reasons, it is not necessary to expose the heart but just requires for delivering a snare into the heart to capture the capture ring 31, and then pull back the snare after the capture is completed, so that the first clip piece 131 and the second clip piece 132 are pulled upwards and opened to release the captured native/natural valve leaflets; and then the snare is pulled back to receive the valve repair device 1 into the delivery sheath for recycling.

In an embodiment, the proximal end of the first clip piece 131 or the second clip piece 132 has an elastic structure, so as to ensure that an elastic restoring force can be provided to the first clip piece 131 or the second clip piece 132 in the case that the traction force at the traction hole is removed. In a specific implementation, the elastic structure can be obtained by performing one or more processes on the piece body of the clip piece, such as trimming, material reducing, bending and the like.

In an embodiment, a bending structure 1314 is formed close to the proximal end of the first clip piece 131 or the second clip piece 132 so as to provide a preset distance between the surface of the first clip piece 131 or the second clip piece 132 and the surface of the first inner clip arm 121 or the second inner clip arm 122. The bending structure 1314 is also used as the elastic structure to provide the elastic restoring force for the clip piece, which is illustrated as the structure in FIG. 13.

In an embodiment, the elastic piece body of the first clip piece 131 or the second clip piece 132 is a piece body with uniform width; in another embodiment, the elastic piece body is a piece body with non-uniform width. Referring to FIG. 15, which is a schematic diagram illustrating a structure of a clip piece in another embodiment of the present disclosure. As illustrated in the figure, for example, the piece body of the first clip piece 131 or the second clip piece 132 is provided with one or more pairs of notch structures, that is, in the present embodiment, the elastic property of the first clip piece 131 or the second clip piece 132 is enhanced by reducing the material of the piece body, and the notch structures are symmetric structures on the piece body so as to guarantee the stress balance thereof.

Referring to FIG. 16, which is an exploded view of an assembled structure of a clip piece and an inner clip arm in an embodiment of the present disclosure. As illustrated in the figure, the first clip piece 131 and the second clip piece 132 are respectively fixed on the plate bodies of the first inner clip arm 121 and the second inner clip arm 122 by welding or riveting. In the above embodiment, each of the first clip piece 131 and the second clip piece 132 includes an elastic piece body 1310 with a hollowed-out structure, a tab 1311 extending from a proximal end of the elastic piece body, and a hangnail-shaped protrusion 1312 formed on a main body of the elastic piece body 1310. Specifically, the elastic piece body 1310, the tab 1311 and the hangnail-shaped protrusion 1312 are all integrally formed on the first clip piece 131 or the second clip piece 132, for example, by cutting, trimming or punching.

Referring to FIG. 17, which is a schematic diagram illustrating an assembled structure of a clip piece and an inner clip arm in an embodiment of the present disclosure. As illustrated in the figure, a part of the first clip piece 131 or the second clip piece 132 close to the proximal end thereof is provided with a tab 1311 extending from the proximal end of the elastic piece body 1310, the tab 1311 has a certain length and a part of the tab 1311 is welded or riveted onto the first inner clip arm 121 or the second inner clip arm 122. Still referring to the case where the first clip piece is fixed on the first inner clip arm 121 by way of example, the tab 1311 of the first clip piece penetrates through the opening 1211 of the first inner clip arm 121 to be engaged on the rear surface of the first inner clip arm 121, and then the tab 1311 is fixed on the rear surface of the first inner clip arm 121 by welding or riveting, while a main part (i.e., the part provided with the puncture needle) of the first clip piece 131 is still located at the front surface of the first inner clip arm 121; in this way, the first clip piece 131 can be fixed on the first inner clip arm 121. Because the tab 1311 described above has certain elastic performance, it can not only fix the first clip piece 131 but also achieve the elastic engagement between the proximal end of the first clip piece 131 and the proximal end of the first inner clip arm 121, so that the first clip piece 131 is elastically closed relative to the first inner clip arm 121. Accordingly, the second clip piece 132 and the second inner clip arm 122 are cooperated with each other in the same way.

In an embodiment, the included angle between the hangnail-shaped protrusion 1312 on the first clip piece 131 or the second clip piece 132 and the elastic piece 1310 is 15°-60°. In the present embodiment, the extending direction of the hangnail-shaped protrusion 1312 is inclined towards the proximal end of the first clip piece 131 or the second clip piece 132 by 15°-60°. Specifically, in some embodiments, the included angle can be 15°, 16°, 17°, 18°, 19°, 20°, 21°, 22°, 23°, 24°, 25°, 26°, 27°, 28°, 29°, 30°, 31°, 32°, 33°, 34°, 35°, 36°, 37°, 38°, 39°, 40°, 41°, 42°, 43°, 44°, 45°, 46°, 47°, 48°, 49°, 50°, 51°, 52°, 53°, 54°, 55°, 56°, 57°, 58°, 59° or 60°.

The above-mentioned angle of the hangnail-shaped protrusion 1312 provides further benefit in that the hangnail-shaped protrusion 1312 can be pulled away from the natural valve leaflets in the case that the first clip piece 131 or the second clip piece 132 can be pulled.

In an embodiment, the length of the hangnail-shaped protrusion 1312 on the first clip piece 131 or the second clip piece 132 is 0.3 mm-3 mm. Specifically, in some embodiments, the length of the hangnail-shaped protrusion 1312 is 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm or 3 mm.

Referring to FIG. 18, which is a schematic diagram illustrating a structure of a hangnail-shaped protrusion on a clip piece in another embodiment of the present disclosure. As illustrated in the figure, the width of the root portion of the hangnail-shaped protrusion on the first clip piece 131 or the second clip piece 132 is smaller than that of the middle portion; that is, each of the hangnail-shaped protrusions 131 extends from the elastic piece body at the above-mentioned included angle and has a width which is smaller at the root portion 13121, and then gradually increases so as to be transited to the middle portion 13122, and then gradually decreases until a sharper needle tip 13123 is formed; in this way, the natural valve leaflets can be held in the gap between the first clip piece 131 or the second clip piece 132 and the first inner clip arm 121 or the second inner clip arm 122 in a better way in the case that the hangnail-shaped protrusions 1312 with the above-described structure penetrate through the natural valve leaflets.

In an embodiment, the hangnail-shaped protrusions distributed on the elastic piece body of the first clip piece 131 or the second clip piece 132 are divided into one group, which includes three hangnail-shaped protrusions or four hangnail-shaped protrusions 1312 arranged close to the distal end of the first clip piece 131 or the second clip piece 132; in another embodiment, such as the embodiment illustrated in FIGS. 13 to 18, the hangnail-shaped protrusions 1312 on the first clip piece 131 or the second clip piece 132 are divided into multiple groups, and the total number of the hangnail-shaped protrusions 1312 in the group closer to the distal end is greater than that of the remaining groups, for example, the total number of the hangnail-shaped protrusions in the group closer to the distal end is three or four, and the total number of the hangnail-shaped protrusion(s) in the remaining groups is one or two.

In an embodiment, in the case that the hangnail-shaped protrusions on the first clip piece 131 or the second clip piece 132 are divided into multiple groups, the multiple groups of hangnail-shaped protrusions are regularly arranged at intervals, for example, the interval may be regular fixed interval or arithmetic gradual interval. In an embodiment, the multiple groups of hangnail-shaped protrusions are also be arranged in a staggered manner, for example, the positions of the hangnail-shaped protrusions are arranged sequentially in a staggered manner.

In an embodiment, the first clip piece 131 or the second clip piece 132 is a double-layered elastic piece (not illustrated in the figure) including an inner elastic piece and an outer elastic piece; the hangnail-shaped protrusions are arranged on the inner elastic piece, and the outer elastic piece is attached onto the inner elastic piece to reinforce the elastic force or clamping force of the clamping piece, and the inner elastic piece is engaged with the outer elastic piece at the proximal end. In the present embodiment, the inner elastic piece can be engaged with the outer elastic piece at the proximal end by welding or riveting; alternatively, the inner elastic piece and the outer elastic piece are an integrally formed structure, which may achieve the engagement of the two elastic pieces at the proximal end by means of bending.

The first clip piece 131 is taken as an example, the first clip piece 131 has a double-layered elastic piece structure including an inner elastic piece and an outer elastic piece. The outer elastic piece is a hollowed-out structure in order to increase the elastic capacity of the outer elastic piece, and the inner elastic piece is provided with an elastic piece body with a hollowed-out structure, a tab extending from the proximal end of the elastic piece body, and hangnail-shaped protrusions formed on the main body of the elastic piece body; herein, the first clip piece 131 in the form of a double-layered elastic piece structure is fixed on the rear surface of the first inner clip arm 121 by welding or riveting the tab extending from the proximal end of the elastic piece of the inner elastic piece. In the present embodiment, the distal end of the first clip piece 131 in the form of a double-layered elastic piece structure is provided with a traction hole; specifically, traction hole is provided at a position of the inner elastic piece corresponding to the outer elastic piece for allowing a traction wire to penetrate therethrough, so as to force the first clip piece 131 to be opened relative to the first inner clip arm 121 by applying the traction force. Correspondingly, the double-layered elastic piece structure of the second clip piece 132 and the arrangement mode thereof may be the same as the first clip piece 131.

In the fixing mechanism for the clip piece assembly of the present disclosure, the tab extending from the proximal end of the elastic piece passes through the opening of the clip plate to be fixed on the rear surface of the clip plate, thus providing a more stable fixing mode. Compared with the fixing mode in the prior art that the tab provided with hangnail-shaped protrusions is fixed on a woven fabric through a collar, the fixing mechanism in the present disclosure is more stable. Furthermore, the fixing mechanism provided by the present disclosure enables the integrally formed clip piece to be arranged on the front and rear surfaces of the clip plate respectively, thereby simplifying the structure in the gap between the clip piece and the clip plate and facilitating capturing the valve and further stably clamping the captured valve, as compared with the prior art where the whole structure of the clip piece is arranged on the front surface of the inner clip arm in the form of a woven structure, which results in the gap between the clip piece and the inner clip arm narrower and is not conducive to capturing and clamping the valve.

The heart is divided into left and right parts, and each part contains a ventricle and an atrium. The two ventricles and two atriums are separated by interventricular septum and atrial septum, respectively. There are valves between each ventricle and atrium to prevent from blood reflux. Normal valves (mitral or tricuspid valves) only allow blood to flow in one direction in the heart. However, in the case that mitral or tricuspid regurgitation occurs, it will cause an increase in atrial pressure, which in turn causes auricular dilatation and expands the valvular annulus, and further leads to an increase in the degree of regurgitation.

At present, in the field of treating mitral or tricuspid regurgitation, the cardiac valves are mostly repaired in an edge-to-edge manner through a transcatheter surgery in order to reduce trauma. The edge-to-edge repair is to connect two valve leaflets that can't be well closed relative to each other through a clamper, so that during systole, the two valve leaflets that couldn't be well closed can be closed relative to each other to eliminate or reduce the mitral or tricuspid regurgitation, while during diastole, the big hole in the native valve is changed to two small holes, which will not affect the opening of the valve leaflets.

With the advent of the aging era, more and more patients suffer from moderate or severe mitral and tricuspid regurgitation, and even some patients have symptoms of calcification and fibrosis, which thickens the valve leaflets. For patients with severe regurgitation, in order to achieve better controlling effect thereto, the clamper needs to grab more valve leaflets. However, the existing valve repair device has limited accommodation space for valve leaflets, which is difficult to be adapted to the patients with hypertrophic valve leaflets or severe regurgitation, thus considerably limiting the application range of products.

According to an embodiment of the present disclosure, a fixing mechanism for a clip piece assembly is provided, including a clip plate and a clip piece. The clip plate includes a first side and a second side opposite to each other along a thickness direction thereof, and the clip plate has an opening. The clip piece includes a first elastic piece and a first tab connected with the first elastic piece, the first elastic piece is located at the first side of the clip plate, and the first tab is configured to extend from the first side, pass through the opening and reach the second side, or, the first tab is configured to be located at the first side of the clip plate. At least one of the first elastic piece and the first tab includes a first arched part.

In the fixing mechanism for the clip piece assembly provided by the embodiment of the present disclosure, by arranging the first arched part on at least one of the first elastic piece and the first tab, the space for accommodating the valve leaflets can be increased on the premise of ensuring the clamping stability, and a good clamping force can be achieved especially for the hypertrophic valve leaflets.

In at least some embodiments, the first elastic piece includes the first arched part; the first elastic piece includes a proximal end and a distal end opposite to each other along its extending direction, and the proximal end is configured to be connected with the first tab; the first arched part is arranged close to the proximal end of the first elastic piece and is arched towards the side away from the first tab in the thickness direction of the first elastic piece.

In at least some embodiments, the first elastic piece includes a first side and a second side which are opposite to each other in the thickness direction, wherein the first side is close to the first tab and the second side is away from the first tab; the first elastic piece further includes a hangnail-shaped protrusion region located at the first side of the first elastic piece, and the clip piece further includes hangnail-shaped protrusions arranged in the hangnail-shaped protrusion region and configured to be abutted against the clip plate; wherein the first arched part is arched towards the second side of the first elastic piece and is located at one side of the hangnail-shaped protrusion region close to the proximal end of the first elastic piece.

In at least some embodiments, the first tab includes the first arched part; the first tab includes a proximal end and a distal end opposite to each other along its extending direction, and the proximal end of the first tab is configured to be connected with the first elastic piece; the first arched part is arranged at the proximal end of the first tab and is arched towards the side away from the first elastic piece in the thickness direction of the first tab.

In at least some embodiments, the clip piece further includes: a second elastic piece, wherein the second elastic piece is located at the first side of the clip plate, and the first elastic piece is located between the clip plate and the second elastic piece in the thickness direction of the clip plate; a second tab stacked with the first tab and connected with the second elastic piece, wherein the second tab is configured to extend from the first side of the clip plate, pass through the opening, and reach the second side of the clip plate, or, the second tab is located at the first side of the clip plate; wherein at least one of the second elastic piece and the second tab includes a second arched part.

In at least some embodiments, the first elastic piece includes the first arched part; the second elastic piece includes the second arched part; the second arched part is arched towards the side away from the second tab in the thickness direction of the second tab, and the outer contours of the second arched part and of the first arched part may be or may not be conformal.

In at least some embodiments, the outer contours of the second arched part and of the first arched part are conformal, and the total number of the first arched part(s) is equal to that of the second arched part(s), so that the second arched part(s) contact(s) and cover(s) the first arched part(s) in one-to-one correspondence.

In at least some embodiments, the total number of the first arched parts is at least two, at least two first arched parts are arranged along the extending direction of the first elastic piece, adjacent two first arched parts have a first spacing distance therebetween in the extending direction of the first elastic piece, and the first arched part has a first length in the extending direction of the first elastic piece; the total number of the second arched parts is at least two, at least two second arched parts are arranged along the extending direction of the second elastic piece, adjacent two second arched parts have a second spacing distance therebetween in the extending direction of the second elastic piece, and the second arched part has a second length in the extending direction of the second elastic piece; wherein the first length is greater than or equal to the first spacing distance, and the second length is greater than or equal to the second spacing distance.

In at least some embodiments, the outer contours of the second arched part and of the first arched part are not conformal, and the total number of the first arched part(s) is greater than that of the second arched part(s), so that a gap is provided between the second arched part(s) and the first arched parts.

In at least some embodiments, the second tab includes the second arched part; the second tab includes a proximal end and a distal end opposite to each other along its extending direction, and the proximal end of the second tab is configured to be connected with the second elastic piece; the second arched part is arranged at the proximal end of the second tab and is arched towards the side away from the second elastic piece in the thickness direction of the second tab.

In at least some embodiments, the first tab includes the first arched part; the second arched part of the second tab is conformal to the first arched part of the first tab.

In at least some embodiments, the clip plate includes a peripheral region surrounding the opening, and the peripheral region includes a third arched part that is arched towards the second side of the clip plate in the thickness direction of the clip plate.

According to another embodiment of the present disclosure, it is further provided a valve repair device including the fixing mechanism for the clip piece assembly as described above.

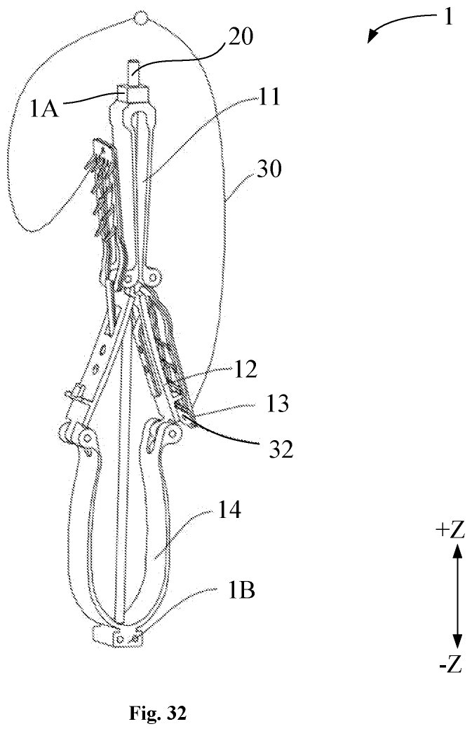

Hereinafter, with reference to FIG. 32 to FIG. 44, the fixing mechanism for the clip piece assembly and the valve repair device thereof provided by the present disclosure would be described through specific embodiments. In order to keep the following description of the embodiments of the present disclosure clear and concise, detailed explanations of known functions and known components may be omitted. In the case that any component of an embodiment of the present disclosure appears in more than one drawing, the component may be represented by the same reference numeral in each drawing.

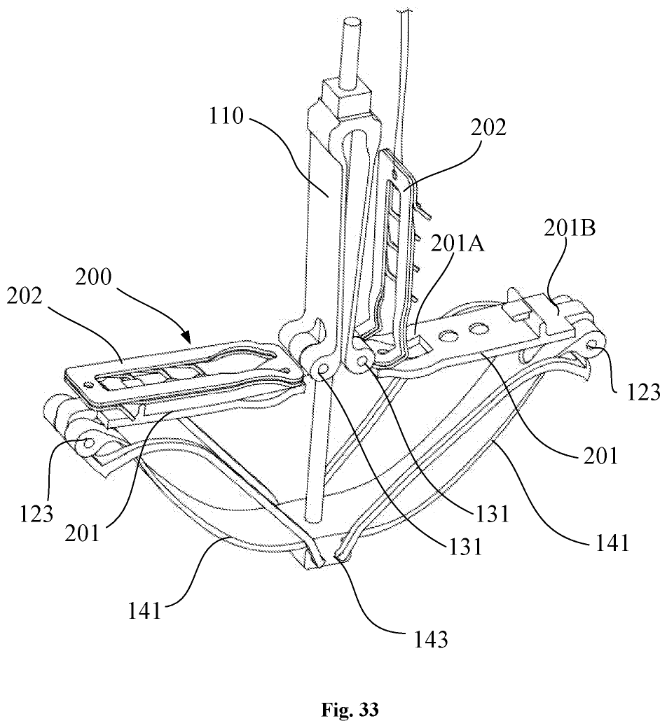

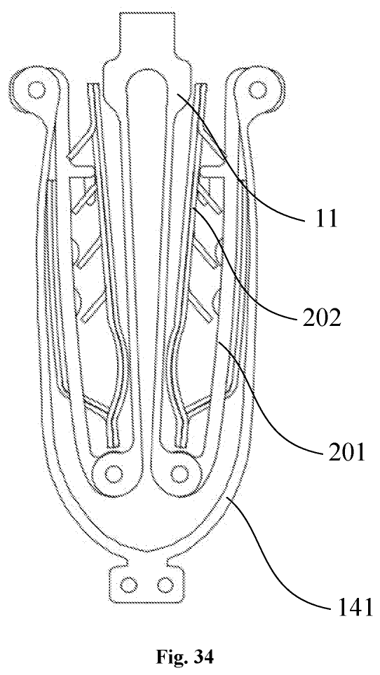

FIG. 32 is a schematic diagram illustrating a structure of a valve repair device provided by an embodiment of the present disclosure in a stretch state. FIG. 33 is a schematic diagram illustrating a structure of a valve repair device provided by an embodiment of the present disclosure in an opened state. FIG. 34 is a schematic diagram illustrating a structure of a valve repair device provided by an embodiment of the present disclosure in a closed state.

As illustrated in FIGS. 32 to 34, the valve repair device 1 can present a plurality of states, such as a stretch state, an opened state and a closed state. The valve repair device 1 can be switched among a plurality of states so as to meet the structural requirements of the valve repair device in the case that the diameter of the delivery device is limited, the valve leaflet is to be grasped and the valve leaflet is to be clamped during the delivery process.

For example, the valve repair device 1 provided according to an embodiment of the present disclosure includes a spacer element 11, an inner clip assembly 12, a clip piece assembly 13 and an outer clip element 14.

For example, the valve repair device 1 includes a proximal end part 1A and a distal end part 1B that are opposite to each other in a longitudinal direction (for example, the Z direction illustrated in the figure). The delivery device includes a driving shaft 20 which is detachably connected with the valve repair device 1. As illustrated in FIG. 32, for example, the driving shaft passes through a through hole in the spacer element 11 and is connected with the distal end part 1B. The driving shaft 20 is configured to be movable in the +Z direction (for example, to be pulled back in the +Z direction). During the delivery process to the target position of the heart by using the delivery device, the valve repair device 1 is in the stretch state illustrated in FIG. 32, that is, FIG. 32 is a structural diagram of the valve repair device 1 in the delivered state.

For example, the inner clip assembly 12 includes two clip plates 201, and the two clip plates 201 are respectively movably connected, such as hinged, with the spacer element 11. In the case that the driving shaft 20 is pulled back in the +Z direction, the distal end part 1B gradually approaches the spacer element 11 in the Z direction, and the two clip plates 201 are forced to be opened in the transverse direction because the position of the spacer element 11 is relatively fixed. At this time, the valve repair device 1 is in an opened state as illustrated in FIG. 33.

For example, the clip piece assembly 13 includes two clip pieces 202 corresponding to two clip plates 201 respectively. One end of each clip piece 202 is connected with the clip plate 201, and the other end is opened relative to the clip plate 201. For example, each clip piece 202 is provided with a traction hole 32, and a traction wire 30 is connected with the traction hole. By pulling the traction wire 30, the opening angle of the clip piece 202 can be controlled, thereby capturing the natural valve leaflet and fixing the captured natural valve leaflet by utilizing the gap between the clip piece 202 and the clip plate 201.

For example, the outer clip element 14 includes two outer clip arms 141 which respectively correspond to two clip plates 201. One end of each of the two outer clip arms 141 is connected with the corresponding clip plate 201, and the other ends of the two outer clip arms 141 are integrally connected with each other (i.e., to form the distal end part 1B), and a clamping space is formed between the two outer clip arms 141. In the case that the captured natural valve leaflets are fixed by the clip pieces 202 and the clip plates 201, the two clip plates 201, the two clip pieces 202 and the spacer element 11 are folded into the clamping space under the elastic restoring force of the two outer clip arms 141. At this time, the valve repair device 1 is in the closed state illustrated in FIG. 34. Finally, by operating the driving shaft 20 to be disengaged from the valve repair device 1, and by pulling the traction wire 30 out of the traction hole 32, the valve repair device 1 is held on the natural valve, and the natural valve thus is clamped.

For example, the valve repair device 1 further includes two fixing mechanisms 200 for the clip piece assembly 13. Since the two fixing mechanisms 200 have the same structure, the following description would be given with reference to one fixing mechanism 200 by way of example.

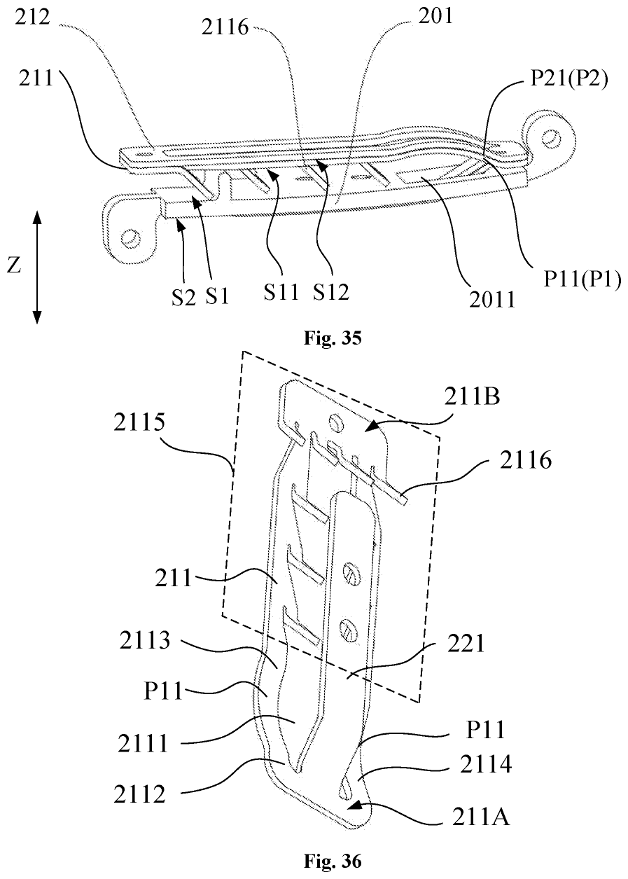

FIG. 35 is a schematic diagram illustrating a structure of a fixing mechanism for a clip piece assembly provided by an embodiment of the present disclosure. Referring to FIGS. 33 to 35, the fixing mechanism 200 includes a clip plate 201 and a clip piece 202.

For example, as illustrated in FIG. 35, the clip plate 201 includes a first side S1 (e.g., the upper side illustrated in the figure) and a second side S2 (e.g., the lower side illustrated in the figure) which are opposite to each other along the thickness direction of the clip plate 201 (e.g., the Z direction illustrated in the figure). The clip plate 201 has an opening 2011, which is a through hole penetrating through the clip plate 201.

For example, the clip piece 202 includes a first elastic piece 211 and a first tab 221 connected with the first elastic piece 211. The first elastic piece 211 is located at the first side S1 of the clip plate 201, and the first tab 221 is configured to extend from the first side S1 to the second side S2 by passing through the opening 2011. By extending the first tab 221 to the second side S2 of the clip plate 201 through the opening 2011, the clamping force between the first elastic piece 211 and the clip plate 201 can be improved with the use of the first tab 221.

In some embodiments, at least one of the first elastic piece 211 and the first tab 221 includes a first arched part P1. That is, the first elastic piece 211 includes the first arched part P1, or the first tab 221 includes the first arched part P1, or both the first elastic piece 211 and the first tab 221 include the first arched part P1. As used herein, the term “arched part” includes an arc-shaped structure, which can be in a shape of a part of a parabola, a circle or an ellipse.

FIG. 36 is a schematic diagram illustrating a structure of a first elastic piece and a first tab according to an embodiment of the present disclosure. For example, as illustrated in FIGS. 35 and 36, the first elastic piece 211 includes a proximal end 211A and a distal end 211B opposite to each other along its extending direction. For example, in the case that the valve repair device 1 is in the opened state, the proximal end 211A is close to the spacer element 11 and the distal end 211B is away from the spacer element 11 (as illustrated in FIG. 33). The proximal end 211A is connected with the first tab 221, and the distal end 211B is not connected with the first tab 221, that is, the distal end 211B is in a suspended state. In this way, a connecting end and an openable end are provided between the first tab 221 and the first elastic piece 211.

For example, the first elastic piece 211 includes a first arched part P11, which is arranged close to the proximal end 211A of the first elastic piece 211 and is arched towards the side away from the first tab 221 in the thickness direction of the first elastic piece 211. That is, the first arched part P11 is close to the connecting end between the first elastic piece 211 and the first tab 221, and is protruded towards an upper side as illustrated in FIG. 35.

In the fixing mechanism 200 of the present disclosure, the natural valve leaflet extends into the gap between the first elastic piece 211 and the first tab 221 through the distal end 211B of the first elastic piece 211. Once the gap between the first elastic piece 211 and the first tab 221 is fixed, the clamping force is also relatively constant. Moreover, the closer to the connecting end, the smaller the gap; while the closer to the openable end, the smaller the clamping force.

The inventor(s) found that, because the natural valve leaflet has a thickness variable depending on each individual, in the case that the clamping force between the first elastic piece 211 and the first tab 221 is suitable for clamping a valve leaflet with smaller thickness, it may exhibit insufficient clamping force for a valve leaflet with greater thickness. In order to improve the clamping force, one of common manners is to reduce the gap between the first elastic piece 211 and the first tab 221, so as to increase the clamping force at the openable end when clamping the valve leaflet. However, the above-mentioned manner further narrows the gap or space at the connecting end, which makes it difficult for the heart valve leaflet to enter the connecting end, resulting in inadequate clamping, especially in the case of native valve leaflet with greater thickness, it is more difficult for the valve leaflet to extend into the elastic clamper to be clamped there.

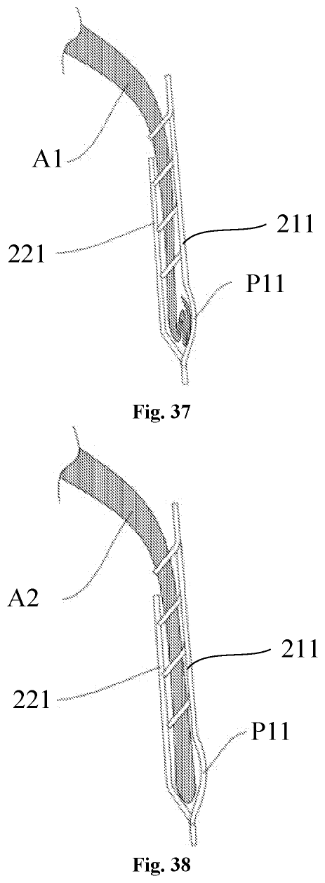

FIG. 37 is a side view of a fixing mechanism provided by an embodiment of the present disclosure which clamps a valve leaflet; FIG. 38 is another side view of a fixing mechanism provided by an embodiment of the present disclosure which clamps a valve leaflet.

As illustrated in FIG. 37 and FIG. 38, in the embodiment of the present disclosure, by providing the first arched part P11 on the first elastic piece 211, the space for accommodating the valve leaflets is increased because the first arched part P11 has an arc-shaped structure which is outwardly convex. In FIG. 37, a part of the native valve leaflet A1 with smaller thickness can be folded for multiple times inside the first arched part P11, so that the first elastic piece 211 is closer to the part of the native valve leaflet A1 having greater thickness, that is, the gap between the first elastic piece 211 and the first tab 221 is not enlarged by the part of the native valve leaflet A1 with smaller thickness, thus ensuring the clamping stability. In FIG. 38, the edge of the native valve leaflet A2 with greater thickness can be also accommodated in the first arched part P11, and the connecting end between the first elastic piece 211 and the first tab 211 is not expanded, thus avoiding an inadequate clamping force at the openable end and ensuring the clamping stability.

Moreover, with the arrangement of the first arched part P11, the distal end 211B of the first elastic piece 211 can be more inclined towards the first tab 221 without changing the position of the connecting end between the first elastic piece 211 and the first tab 221, so as to obtain a greater clamping force at the openable end, and to ensure that the whole first elastic piece 211 has a greater clamping force from the proximal end to the distal end.

For example, as illustrated in FIG. 35, the first elastic piece 211 includes a first side S11 and a second side S12 opposite to each other in the thickness direction thereof (for example, the Z direction illustrated in the figure), the first side S11 is close to the first tab 221, and the second side S12 is away from the first tab 221. As illustrated in FIG. 36, the first elastic piece 211 further includes a hangnail-shaped protrusion region 2115 located at the first side S11 of the first elastic piece 211, and the clip piece 202 further includes hangnail-shaped protrusions 2116 which are arranged in the hangnail-shaped protrusion region 2115 and configured to be abutted against the clip plate 201. With the arrangement of the hangnail-shaped protrusions, it is more beneficial to capture the valve leaflets and fix the captioned valve leaflets between the first elastic piece 211 and the clip plate 201.

For example, as illustrated in FIGS. 35 and 36, the first arched part P11 is arched towards the second side S12 of the first elastic piece 211 and is located at the side of the hangnail-shaped protrusion region 2115 close to the proximal end 211A of the first elastic piece 211. In the embodiment of the present disclosure, by arranging the first arched part P11 at the side of the hangnail-shaped protrusion region 2115 close to the proximal end 211A of the first elastic piece 211, the space for accommodating the valve leaflets can be increased without changing the existing design of the hangnail-shaped protrusion region, thereby reducing the manufacturing difficulty.

For example, as illustrated in FIG. 36, the first elastic piece 211 includes a first hollowed-out region 2111 and a first peripheral region 2112 surrounding the first hollowed-out region 2111. The first peripheral region 2112 includes a first portion 2113 and a second portion 2114 both extending along the extending direction of the first elastic piece 211, and the first hollowed-out region 2111 is located between the first portion 2113 and the second portion 2114. With the arrangement of the first hollowed-out region 2111, on one hand, the elastic force of the first elastic piece can be enhanced, which is beneficial to capture and fix the valve leaflet; and on the other hand, the overall weight of the fixing mechanism or the valve repair device can be reduced as compared with the case that the first elastic piece 211 is a solid structure.

In some embodiments, the first arched part P11 is arranged on at least one of the first portion 2113 and the second portion 2114. That is, the first arched part P11 may be arranged on the first portion 2113, or, the first arched part P11 may be arranged on the second portion 2114, or, the first arched part P11 may be arranged on both the first portion 2113 and the second portion 2114.

For example, as illustrated in FIG. 36, the first arched part P11 is arranged on both the first portion 2113 and the second portion 2114. With the arrangement of the first arched part P11 on both the first portion 2113 and the second portion 2114, more valve leaflets can be accommodated without affecting the overall weight of the fixing mechanism or the valve repair device.

In the embodiment of the present disclosure, the clip piece 202 may be single-layered or multi-layered, for example, it may be a double-layered structure. For example, as illustrated in FIGS. 32 to 35, the clip piece 202 is a double-layered structure. In the case that the clip piece 202 is designed in the form of a stack of a first elastic piece 211 and a second elastic piece 212, the thickness of the elastic piece can be reduced, and the elasticity of the elastic piece can be improved on the premise of ensuring the strength of the elastic piece. When grasping the valve leaflet, the elastic piece and the clip plate can be opened relative to each other by a larger angle to facilitate capturing the valve leaflet.

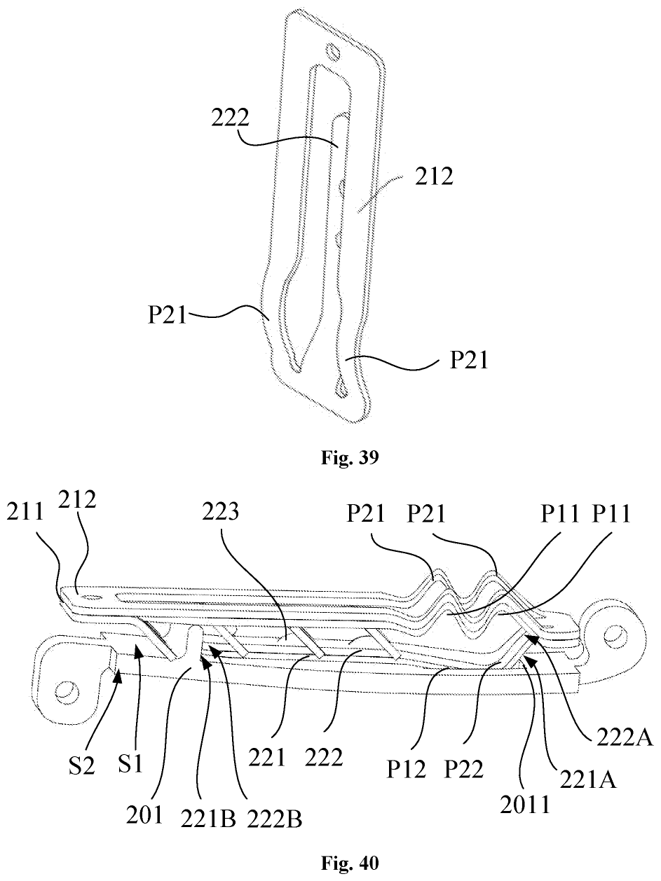

FIG. 39 is a schematic diagram illustrating a structure of a second elastic piece and a second tab provided by an embodiment of the present disclosure.

For example, as illustrated in FIGS. 35 and 39, the clip piece 202 further includes a second elastic piece 212 and a second tab 222. The second elastic piece 212 is located at the first side S1 of the clip plate 201, and the first elastic piece 211 is located between the clip plate 201 and the second elastic piece 212 in the thickness direction of the clip plate 201, and the second tab 222 is stacked with the first tab 221. That is, in the Z direction, the second elastic piece 212 is laminated on the first elastic piece 211, and the second tab 222 is laminated on the first tab 221. For example, the second tab 222 is connected with the second elastic piece 212, and the second tab 222 extends from the first side S1 of the clip plate 201 to the second side S2 of the clip plate 201 through the opening 2011.

In the embodiment of the present disclosure, by arranging the second elastic piece 212 on the first elastic piece 211 and arranging the second tab 222 on the first tab 221, the elasticity of the first elastic piece and the first tab can be enhanced.

In some embodiments, at least one of the second elastic piece 212 and the second tab 222 includes a second arched part P2. That is, the second elastic piece 212 includes the second arched part P2, or, the second tab 222 includes the second arched part P2, or, both the second elastic piece 212 and the second tab 222 include the second arched part P2.

For example, as illustrated in FIGS. 35 and 39, the second elastic piece 212 includes a second arched part P21. By arranging the second arched part P21 on the second elastic piece 212, the elastic restoring force of the first arched part P11 can be enhanced without affecting the existing design of the first arched part P11.

In some embodiments, the second arched part P21 is arched towards the side away from the second tab 222 in the thickness direction of the second tab 222, and the outer contours of the second arched part P21 and of the first arched part P11 may or may not be conformal.

For example, as illustrated in FIGS. 35 and 39, the outer contours of the second arched part P21 and of the first arched part P11 are conformal, that is, the arc-shaped structures of the second arched part P21 and of the first arched part P11 have the same radian, so that the second arched part P21 covers and is tightly attached onto the first arched part P11, which further enhances the elastic restoring force of the first arched part P11.

In the embodiments illustrated in FIGS. 32 to 39, the first elastic piece 211 and the second elastic piece 212 are respectively provided with the first arched part P11 and the second arched part P21 thereon, but the first tab 221 and the second tab 222 are not provided with any arched part. However, it can be understood that in some other embodiments of the present disclosure, the first tab 221 may be further provided with the first arched part P11 thereon, and the second tab 222 may be further provided with the second arched part P21 thereon.

FIG. 40 is a schematic diagram illustrating a structure of a fixing mechanism provided by another embodiment of the present disclosure.

For example, as illustrated in FIG. 40, the fixing mechanism 200 includes a clip plate 201 and a clip piece 202, wherein the clip piece 202 includes a first elastic piece 211, a first tab 221, a second elastic piece 212 and a second tab 222.

For example, the first tab 221 includes the first arched part P12, and the first tab 221 includes a proximal end 221A and a distal end 221B opposite to each other along its extending direction, and the proximal end 221A of the first tab 221 is connected with the first elastic piece 211. The first arched part P11 is arranged at the proximal end 221A of the first tab 221 and is arched towards the side away from the first elastic piece 211 in the thickness direction of the first tab 221. That is, the first arched part P12 is close to the connecting end between the first tab 221 and the first elastic piece 211, and is arched towards a lower side as illustrated in the figure. By arranging the first arched part P12 on the first tab 221, the space for accommodating the valve leaflets can be further increased.

For example, the first tab 221 is located at the first side S1 of the clip plate 201. That is, at least part of the first tab 221 is fixed at the first side S1 of the clip plate 201, for example, at least part of the first tab 221 is fixedly connected to the clip plate 201 by means of two rivets 223.

Compared with the case where the first tab 221 passes through the clip plate 201 as illustrated in FIG. 35, by arranging the first tab 221 at the first side S1 of the clip plate 201, the height of the first tab 221 in the thickness direction of the clip plate 201 can be reduced on the premise of keeping approximately the same accommodation space. In this way, in the case that the first elastic piece 211 is opened, the force generated by the first tab 221 against deformation is smaller.

For example, the second tab 222 includes the second arched part P22; the second tab 222 includes a proximal end 222A and a distal end 222B opposite to each other along its extending direction, and the proximal end 222A of the second tab 222 is connected with the second elastic piece 212. The second arched part P22 is arranged at the proximal end 222A of the second tab 222 and is arched towards the side away from the second elastic piece 212 in the thickness direction of the second tab 222. That is, the second arched part P22 is close to the connecting end between the second tab 222 and the second elastic piece 212, and is arched towards a lower side as illustrated in the figure. By arranging the second arched part P22 on the second tab 222, the elastic restoring force of the first arched part P11 can be enhanced without affecting the existing design of the first arched part P11.

For example, the second tab 222 is located at the first side S1 of the clip plate 201, and the first tab 221 is located between the clip plate 201 and the second tab 222 in the thickness direction of the clip plate 201. That is, at least part of the second tab 222 is fixed at the first side S1 of the clip plate 201, for example, both of the first tab 221 and the second tab 22 are fixedly connected to the clip plate 201 by means of two rivets 223.

Compared with the case where the second tab 222 passes through the clip plate 201 in FIG. 35, by arranging the second tab 222 at the first side S1 of the clip plate 201, the length of the second tab 222 in its extending direction can be increased and the height of the second tab 222 in the thickness direction of the clip plate 201 can be reduced on the premise of keeping approximately the same accommodation space. In this way, in the case that the first elastic piece 211 is opened, the force generated by the second tab 222 against deformation is smaller. Moreover, by arranging the second tab 222 on the first tab 221, the elastic restoring force of the first tab 221 can be improved.

For example, as illustrated in FIG. 40, at least part of the first arched part P12 is located in the opening 2011, and at least part of the second arched part P22 is located in the opening 2011. With the above arrangement, the overall structure of the fixing mechanism 200 can be made smaller and more compact on the premise of providing a larger space for accommodating valve leaflets.

For example, the outer contours of the second arched part P22 and of the first arched part P12 are conformal. That is, the arc-shaped structures of the second arched part P21 and of the first arched part P11 have the same radian, so that the second arched part P21 covers and is closely attached onto the first arched part P11, which can further enhance the elastic restoring force of the first arched part P11.

In some embodiments, a plurality of first arched parts P11 are provided on the first elastic piece 211 and a plurality of second arched parts P21 are provided on the second elastic piece 212. For example, the plurality of first arched parts P11 constitute a wavy structure, and the plurality of second arched parts P21 also constitute a wavy structure.

For example, as illustrated in FIG. 40, the first elastic piece 211 includes at least two first arched parts P11, and at least two the first arched parts P11 are arranged along the extending direction of the first elastic piece 211 (for example, the lateral direction illustrated in the figure). For example, the second elastic piece 212 includes at least two second arched parts P21, and at least two the second arched parts P21 are arranged along the extending direction of the second elastic piece 212 (for example, the lateral direction illustrated in the figure).

For example, the first elastic piece 211 and the second elastic piece 212 can be connected with each other through a covering film wrapped outside the first elastic piece 211 and the second elastic piece 212 but are not completely fixed with each other. The first elastic piece 211 and the second elastic piece 212 are movable relative to each other during the opening or closing of the clip pieces.

For example, there is no hangnail-shaped protrusion on the second elastic piece 212, which on one hand can avoid the dislocation of hangnail-shaped protrusions caused by the relative movement between the first elastic piece 211 and the second elastic piece 212 during the opening process of the elastic pieces. On the other hand, the valve leaflet is punctured only by the hangnail-shaped protrusions on the first elastic piece 211 so that the thickness of the hangnail-shaped protrusion can be reduced and the puncture force of the hangnail-shaped protrusion applied on the valve leaflet can be improved.

Compared with the case illustrated in FIG. 35 where only one first arched part P11 and one second arched part P21 are provided, in the embodiment of the present disclosure, by arranging at least two first arched parts P11 and at least two second arched parts P21, on the one hand, the accommodation space for valve leaflets can be further improved, and on the other hand, suitable accommodation space can be provided for valve leaflets with different lengths. For example, in the case that the valve leaflet is longer, the redundant part of the valve leaflet can be accommodated in the accommodation space at the right side of the figure; in the case that the valve leaflet is shorter, the redundant part of the valve leaflet can be accommodated in the accommodation space at the left side of the figure.

For example, the outer contours of at least two first arched parts P11 and of the at least two second arched parts P21 are conformal, so that the elastic restoring force of the at least two first arched parts P11 can be enhanced with the aid of the at least two second arched parts P21. The total number of the first arched parts P11 is equal to the total number of the second arched parts P21, so that the second arched parts P21 contact with and cover the first arched parts P21 in one-to-one correspondence, thereby further enhancing the elastic restoring force of the at least two first arched parts P11.

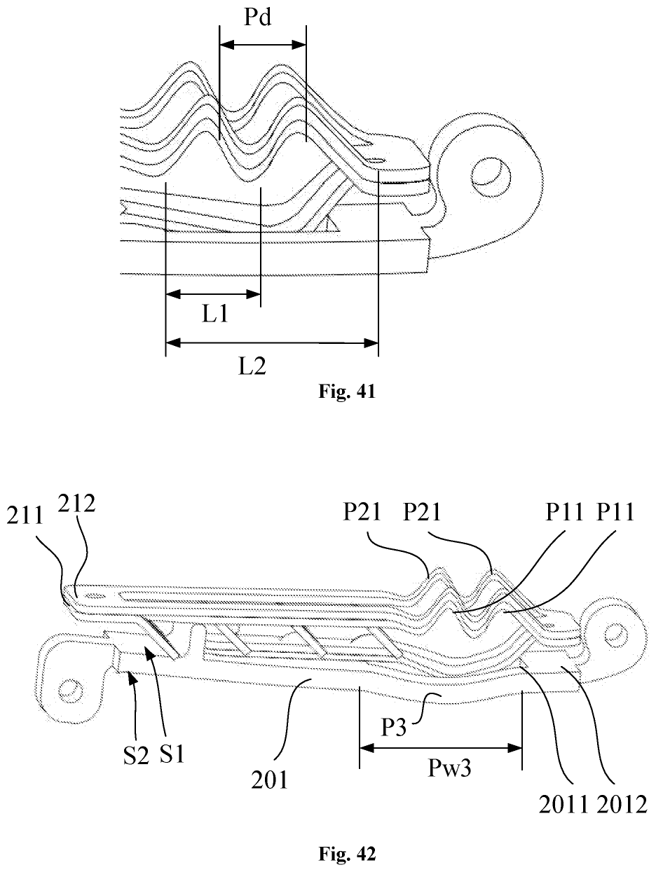

FIG. 41 is a schematic diagram illustrating a partial structure of the fixing mechanism of FIG. 40. As illustrated in FIG. 41, two adjacent first arched parts P11 have a first spacing distance Pd in the extending direction of the first elastic piece 211, and each of the first arched parts P11 has a first length L1 in the extending direction of the first elastic piece 211; the first length L1 is greater than or equal to the first spacing distance Pd; in this way, more valve leaflets can be accommodated. Preferably, the first length L1 is greater than the first spacing space Pd.

It can be understood that two adjacent second arched parts P21 also have a second spacing distance (not illustrated) in the extending direction of the second elastic piece 212, and each of the second arched parts P21 has a second length (not illustrated) in the extending direction of the second elastic piece 212, and the second length is greater than or equal to the second spacing distance; in this way, the elastic restoring force of the at least two first arched parts P11 can be enhanced under the condition that more valve leaflets can be accommodated.

For example, as illustrated in FIG. 41, the sum L2 of the lengths of the two first arched parts P11 in the extending direction of the first elastic piece 211 is smaller than or equal to 30% of the total length of the first elastic piece 211, which can ensure that the portion of the first elastic piece 211 for accommodating the valve leaflets can be fully opened when capturing the valve leaflets.

FIG. 42 is a schematic diagram illustrating a structure of a fixing mechanism provided by yet another embodiment of the present disclosure. The difference between FIG. 42 and FIG. 40 lies in that the clip plate 201 includes a third arched part P3.

For example, as illustrated in FIG. 42, the clip plate 201 includes a peripheral region 2012 surrounding the opening 2011, and the peripheral region 2012 includes a third arched part P3 which is arched towards the second side S2 of the clip plate 201 in the thickness direction of the clip plate 201. That is, the third arched part P3 is protruded towards a lower side as illustrated in the figure. With the arrangement of the third arched part P3, because the third arched part P3 of the clip plate 201 is close to the connecting end between the first elastic piece and the first tab, the space for accommodating the valve leaflets can be further increased, and the valve leaflets can be extended to the connecting end to ensure proper clamping, as compared with the case of FIG. 40.

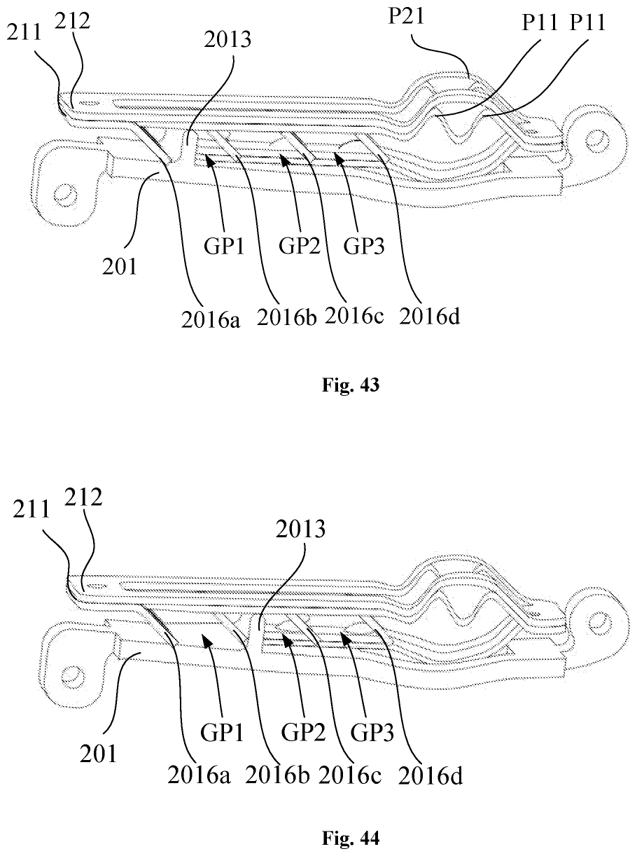

FIG. 43 is a schematic diagram illustrating a structure of a fixing mechanism provided by further another embodiment of the present disclosure. The difference between FIG. 43 and FIG. 42 lies in that the first elastic piece 211 includes at least two first arched parts P11, and the second elastic piece 212 includes one second arched part P21.

In some embodiments, the outer contours of the second arched part P21 and of the first arched part P11 are not conformal, and the total number of the first arched part(s) P11 is greater than that of the second arched part(s) P21, so that a gap is provided between the second arched part(s) P21 and a plurality of first arched parts P11. For example, as illustrated in FIG. 43, at least two first arched parts P11 constitute a wavy structure, and the second arched part P21 constitutes a single arc-shaped structure, so that a gap is formed therebetween. With the above arrangement, the second elastic piece 212 can also play a role of enhancing the elastic restoring force of the first elastic piece 211, so as to jointly clamp the valve leaflets.

FIG. 44 is a schematic diagram of a fixing mechanism provided by yet further another embodiment of the present disclosure. The difference between FIG. 44 and FIG. 43 lies in the position of the limiting block 2013 on the clip plate 201.

In FIG. 43, the clip plate 201 further includes a limiting block 2013, which is configured to be abutted against a clip piece, such as the first elastic piece 211, so as to maintain a gap between the clip piece 202 (such as the first elastic piece 211) and the clip plate 201 for accommodating the valve leaflets.

In FIG. 43, a plurality of rows of hangnail-shaped protrusions (for example, four rows 2116a to 2116d illustrated in the figure) are arranged on the first elastic piece 211, and each row of hangnail-shaped protrusions includes a plurality of hangnail-shaped protrusions 2116. Among the plurality of rows of hangnail-shaped protrusions, there is a gap between every two adjacent rows of hangnail-shaped protrusions, such as the first gap GP1, the second gap GP2 and the third gap GP3. In the extending direction of the first elastic piece 211, the first gap GP1 is farthest away from the first arched part P11 of the first elastic piece 211, and the third gap GP3 is closest to the first arched part P11 of the first elastic piece 211. In FIG. 43, the limiting block 2013 is located in the first gap GP1.

In some embodiments, the limiting block 2013 may be located in other gaps except the first gap GP1. For example, as illustrated in FIG. 44, the limiting block 2013 may be located in the second gap GP2 or the third gap GP3.

Compared with the case of providing the limiting block in the first gap GP1, by arranging the limiting block 2013 in the second gap GP2 or the third gap GP3, it can further improve the clamping force between the clip piece and the clip plate on the premise of ensuring that there is a gap between the clip piece and the clip plate to accommodate the valve leaflets, thus improving the clamping stability.

In some embodiments, as illustrated in FIG. 32, which is a structural diagram of a valve repair device in a stretch state, that is, a delivered state. The spacer element 11 includes a spacer body 110 with a first stiffness, that is, the stiffness of the main part of the spacer element 11 is defined as the first stiffness. The spacer element 11 moves in the −Z direction under the force output by the driving shaft 20 of the delivery device. During the movement, the spacer element 11 itself would not be deformed because of its first stiffness.

In some embodiments, the two clip plates 201 of the inner clip assembly 12 have a second stiffness, respectively. For example, as illustrated in FIG. 33, each of the clip plates 201 includes a proximal end 201A and a distal end 201B opposite to each other in the extending direction thereof, herein the proximal end 201A is close to the spacer element 11 and the distal end 201B is away from the spacer element 11. The proximal end 201A is movably connected with the spacer body 110, for example, through a first hinge part 131; the distal end 201B is movably connected with the outer clip arm 141, for example, through a second hinge part 123. In the case that the valve repair device is delivered to the target position, it is changed from the stretch state to the opened state by pulling back the driving shaft 20 through an external handle; because the proximal end of the spacer element 11 is fixed on the external delivery device, the position of the spacer element 11 remains unchanged, so that the proximal end 201A and the distal end 201B of the clip plate 201 would be applied with forces from the spacer element 11 and the outer clip element 14 respectively, in this situation, the clip plate 201 itself would not be deformed because the clip plate 201 has the second stiffness.

As illustrated in FIG. 32 and FIG. 33, during the movement of the driving shaft 20 along the +Z direction, because the spacer element 11 itself would not be deformed and the spacer body 110 is movably connected with the two clip plates 201 respectively, the two clip plates 201 are gradually opened from the initial, basically vertical state in FIG. 32 to the basically horizontal state in FIG. 33; at the same time, because the two outer clip arms 141 of the outer clip element 14 are movably connected with the two clip plates 201 respectively, and because the position of the spacer element 11 is relatively fixed, the space of the valve repair device in the Z direction is compressed during the movement of the driving shaft 20 in the +Z direction, and the spacing distance between the two clip plates 201 and between the two outer clip arms 141 is forced to be gradually increased in the transverse direction; that is, the two clip plates 201 and the two outer clip arms 141 are simultaneously opened towards both sides (outwardly), which presents an inverted umbrella shape. At the side where the outer clip element 14 is connected with the clip plate 201, the strength of the outer clip element 14 is smaller than that of the clip plate 201.

In the embodiment of the present disclosure, in the case that the two clip plates 201 are basically in the same plane (for example, the state illustrated in FIG. 33), the valve repair device is in an opened state and ready for capturing the valve leaflet. When the clip piece 202 is pulled upwardly, it may be opened to have an included angle approximately 90 degrees relative to the clip plate 201 due to its relatively greater elasticity, which is not only beneficial to capture the valve leaflet, but also beneficial to allow the valve leaflet to extend as close as possible to the proximal end 201A of the clip plate 201 according to the patient's condition, so as to ensure the clamping stability. After the valve leaflet reaches a proper position, the clip piece 202 is released to close, and the clip piece 202 and the clip plate 201 jointly clamp the valve leaflet.

In some embodiments, as illustrated in FIG. 32 and FIG. 33, two outer clip arms 141 of the outer clip element 14 are integrally formed with the distal end part 143, and the two outer clip arms 141 have a third stiffness respectively. Because the outer clip arms 141 have the third stiffness, when it is applied with an acting force by the two clip plates 201, the side of the outer clip arm 141 close to the clip plates 201 is forced to be opened outwardly.

For example, the two outer clip arms 141 have a U-shaped configuration. In the case that the two clip plates 201 are located in the same plane, the two outer clip arms 141 are pushed to be opened to have a maximum distance between the proximal ends of the two outer clip arms. At this time, the valve repair device 1 presents an inverted umbrella-like shape.

For example, the stiffness distribution of each of the outer clip arms 141 close to the second hinge part 123 is smaller than that close to the distal end part 143, in this way, in the case that the two outer clip arms 141 are in an opened state, the outer clip arms 141 located at the distal end part 143 can provide a restoring force for the valve repair device to converge inwardly.

As illustrated in FIG. 33 and FIG. 34, in the case that the driving shaft 20 continues to move in the +Z direction, due to the elastic restoring force of the two outer clip arms 141 per se and the pulling force applied on the outer clip arms 141 by the driving shaft 20, the distal ends 143 of the outer clip arms 141 continue to move towards the spacer body 110 and the two distal ends 201B of the two clip plates 201 converge inwardly, so that the spacer element 11, the inner clip assembly 12 and the clip piece assembly 13 are all folded into the clamping space between the two outer clip arms 141 to achieve the closed state illustrated in FIG. 34. At this time, the two valve leaflets being clamped are close to the spacer body 110 to form a connecting point of the valve leaflets. Because there is no gap at the connecting point, the phenomenon of regurgitation can be better avoided after clamping the valve leaflets (the valve leaflets are not illustrated in the figure).

In some embodiments, the two clip pieces 202 have a fourth stiffness, respectively. Because the two clip pieces 202 have the fourth stiffness, when they are applied with a force by the traction wire 30, the middle portions of the two clip pieces 202 slightly bend due to their own elasticity. However, in the case that the valve repair device 1 is switched between different states, the two clip pieces 202 themselves would not be deformed because no force is applied thereon.

In some embodiments, the second stiffness of the clip plate 201 may be the same as or different from the first stiffness of the spacer body 110. In the case that the second stiffness is different from the first stiffness, for example, the second stiffness is greater than the first stiffness, the clip plate 201 can bear a force greater than the spacer body 110. That is to say, the strength of the clip plate 201 and the overall stability of the valve repair device are improved, which thus is preferable.

In some embodiments, the second stiffness of the clip plate 201 is greater than the third stiffness of the outer clip arm 141. With the above arrangement, it is beneficial to apply a certain force to the outer clip arms 141 while the clip plates 201 are opened outwardly to be located in the same horizontal plane, so that the two outer clip arms 141 can be opened outwardly.

In some embodiments, the fourth stiffness is smaller than the third stiffness. In this way, the two clip pieces 202 are more flexible and elastic than the two outer clip arms 141. In the case that the valve leaflet to be clamped is relatively thicker, the clip pieces 202 can be cooperated with the clip plates 201 to clamp the valve leaflet in a better way due to the excellent flexibility and elasticity of the clip pieces 202, which further improves the clamping stability.

In this paper, the term “stiffness” refers to the ability of a structure to resist elastic deformation in the case that it is applied with a force, and it is used to characterize the difficulty of structural deformation or the external force required for structural deformation. For example, the stiffness of a spacer body refers to the external force required for the deformation of the spacer body, the stiffness of a clip plate refers to the external force required for the deformation of the clip plate, and the stiffness of an outer clip arm refers to the external force required for the deformation of the outer clip arm. It should be understood that the stiffness of the outer clip arm is smaller than that of the inner clip arm, which means the force suffered by the outer clip arm when deformed is not sufficient to cause the inner clip arm to have a deformation even it's applied on the inner clip arm. The stiffness of the main part of the spacer element refers to the ability of the main part of the spacer element to resist elastic deformation when applied with a force.

In the fixing mechanism for the clip piece assembly and the valve repair device provided by the embodiments of the present disclosure, by arranging the first arched part on at least one of the first elastic piece and the first tab, the space for accommodating the valve leaflet can be increased on the premise of ensuring the clamping stability, especially for patients with hypertrophic (larger) valve leaflets or severe regurgitation, and a good clamping force can be achieved to improve the regurgitation repairing performance. Furthermore, at least one of the second elastic piece 212 and the second tab 222 includes the second arched part P2, so that the elastic restoring force of the first elastic piece 211 can be further enhanced. Moreover, by arranging the third arched part P3 on the clip plate, the space for accommodating the valve leaflet can be further increased.

The above-described embodiments only illustrate the principle and effects of the present disclosure, and are not intended to limit the present disclosure. Anyone familiar with this technology can modify or change the above embodiments without violating the spirit and scope of the present disclosure. Therefore, all equivalent modifications or changes made by people with ordinary knowledge in the technical field without departing from the spirit and technical ideas disclosed in the present disclosure should still be covered by the claims of the present disclosure.

In the disclosure, the following should be noted:

(1) The accompanying drawings involve only the structure(s) in connection with the embodiment(s) of the present disclosure, and other structure(s) can be referred to common design(s).

(2) For the purpose of clarity only, in accompanying drawings for illustrating the embodiment(s) of the present disclosure, the thickness and a size of a layer or area may be enlarged or narrowed, that is, the drawings are not drawn in a real scale.

(3) In case of no conflict, features in one embodiment or in different embodiments can be combined as a new embodiment.

1. A valve repair device, comprising:
a spacer element, comprising a proximal end and a distal end opposite to each other in an extending direction thereof, wherein the proximal end is configured to be connected to a delivery device, and the distal end comprises two end portions opposite to each other;
an inner clip assembly, comprising a first inner clip arm and a second inner clip arm, wherein each of the first inner clip arm and the second inner clip arm comprises a proximal end and a distal end opposite to each other in an extending direction thereof, the proximal end is close to the spacer element and the distal end is away from the spacer element;
a clip piece assembly, wherein at least part of the clip piece assembly is arranged on the inner clip assembly, and the clip piece assembly comprises a first clip piece rotatable relative to the first inner clip arm and a second clip piece rotatable relative to the second inner clip arm;
an outer clip element, comprising a distal end part, a first outer clip arm and a second outer clip arm both connected with the distal end part, and a clamping space located between the first outer clip arm and the second outer clip arm, wherein each of the first outer clip arm and the second outer clip arm comprises a proximal end and a distal end opposite to each other in an extending direction thereof, and the proximal end is close to the clip piece assembly, and the distal end is away from the clip piece assembly;
a first hinge structure, configured to connect the two end portions to the proximal end of the first inner clip arm and the proximal end of the second inner clip arm, respectively;
a second hinge structure configured to connect the distal end of the first inner clip arm and the distal end of the second inner clip arm to the proximal end of the first outer clip arm and the proximal end of the second outer clip arm, respectively;
wherein in the state that the spacer element, the clip piece assembly and the inner clip assembly are located in the clamping space, the first outer clip arm and the second outer clip arm maintain opposite clamping forces.

2. The valve repair device according to claim 1, wherein,
the two end portions comprise a first end portion and a second end portion; and the first hinge structure comprises two first hinge parts;
one of the two first hinge parts is arranged at the first end portion and is configured to hinge the first end portion with the proximal end of the first inner clip arm;
the other one of the two first hinge parts is arranged at the second end portion and is configured to hinge the second end portion with the proximal end of the second inner clip arm.

3. The valve repair device according to claim 2, wherein the spacer element comprises:
a connector head, located at the proximal end of the spacer element;
a neck part, configured to be connected with the connector head, wherein the neck part comprises a bridge structure; and
a first spacer plate and a second spacer plate, configured to be respectively connected with the bridge structure and extend from the bridge structure to the distal end of the spacer element, wherein the first spacer plate comprises one of the two end portions, and the second spacer plate comprises the other one of the two end portions.

4. The valve repair device according to claim 3, wherein,
a gap is provided between the first spacer plate and the second spacer plate;
the delivery device comprises a driving shaft configured to pass through the connector head and the bridge structure and extend to the distal end part through the gap;
the gap has a spacing distance in a direction perpendicular to an extending direction of the driving shaft, and the spacing distance gradually decreases in the extending direction of the driving shaft and in a direction close to the distal end part.

5. The valve repair device according to claim 3, wherein the spacer element further comprises:
a connecting part, wherein the connecting part is arranged at the connector head, and the connecting part comprises a through hole and ear-shaped blocks located at two opposite sides of the connecting part, and the ear-shaped blocks are configured to be snap-fitted with the delivery device so that the driving shaft of the delivery device passes through the through hole.

6. The valve repair device according to claim 3, wherein the neck part, the first spacer plate, the second spacer plate and the two first hinge parts are integral structure.

7. The valve repair device according to claim 1, wherein,
the second hinge structure comprises two second hinge parts;
one of the two second hinge parts is arranged at the distal end of the first inner clip arm and is configured to hinge the distal end of the first inner clip arm with the proximal end of the first outer clip arm;
the other one of the two second hinge parts is arranged at the distal end of the second inner clip arm and is configured to hinge the distal end of the second inner clip arm with the proximal end of the second outer clip arm.

8. The valve repair device according to claim 7, wherein,
the first inner clip arm comprises a first clip plate, and the first clip piece is configured to be opened or closed relative to the first clip plate;
the second inner clip arm comprises a second clip plate, and the second clip piece is configured to be opened or closed relative to the second clip plate;
wherein at least one clip plate of the first clip plate and the second clip plate has an opening, and a part of the first clip piece or the second clip piece corresponding to the at least one clip plate is configured to extend from one side surface of the at least one clip plate to the other side surface of the at least one clip plate through the opening.

9. The valve repair device according to claim 7, wherein,
the first inner clip arm comprises a first clip plate, and the first clip piece is configured to be opened or closed relative to the first clip plate;
the second inner clip arm comprises a second clip plate, and the second clip piece is configured to be opened or closed relative to the second clip plate;
at least one clip plate of the first clip plate and the second clip plate is provided with a fixing hole, which is configured to fix a part of the clip piece corresponding to the at least one clip plate on the at least one clip plate.

10. The valve repair device according to claim 7, wherein the inner clip assembly further comprises a limiting block, the limiting block is located on at least one inner clip arm of the first inner clip arm and the second inner clip arm and configured to maintain a gap between the at least one inner clip arm and the first clip piece or the second clip piece corresponding to the at least one inner clip arm.

11. The valve repair device according to claim 7, wherein the inner clip assembly further comprises a puncture hole or a puncture groove, the puncture hole or the puncture groove is located on at least one inner clip arm of the first inner clip arm and the second inner clip arm, and is configured to be engaged with a hangnail-shaped protrusion on the first clip piece or the second clip piece corresponding to the at least one inner clip arm.

12. The valve repair device according to claim 1, wherein,
at least one inner clip arm of the first inner clip arm and the second inner clip arm comprises a clip plate, wherein the clip plate comprises a first side and a second side opposite to each other along a thickness direction thereof, and the clip plate is provided with an opening;
at least one clip piece of the first clip piece and the second clip piece is configured to be opened or closed relative to the clip plate;
the at least one clip piece comprises a first elastic piece and a first tab connected with the first elastic piece, wherein the first elastic piece is located at the first side of the clip plate, and the first tab is configured to extend from the first side to the second side through the opening, or, the first tab is located at the first side of the clip plate;
wherein at least one of the first elastic piece and the first tab comprises a first arched part.

13. The valve repair device according to claim 12, wherein:
the first elastic piece comprises the first arched part, and the first elastic piece comprises a proximal end and a distal end opposite to each other along an extending direction thereof, and the proximal end is configured to be connected with the first tab;
the first arched part is arranged close to the proximal end of the first elastic piece and is arched towards a side facing away from the first tab in a thickness direction of the first elastic piece.

14. The valve repair device according to claim 12, wherein,
the first tab comprises the first arched part, the first tab comprises a proximal end and a distal end opposite to each other along an extending direction thereof, and the proximal end of the first tab is configured to be connected with the first elastic piece;
the first arched part is arranged at the proximal end of the first tab and is arched towards a side facing away from the first elastic piece in a thickness direction of the first tab.

15. The valve repair device according to claim 12, wherein the clip piece further comprises:
a second elastic piece, located at the first side of the clip plate, wherein the first elastic piece is provided between the clip plate and the second elastic piece in a thickness direction of the clip plate;
a second tab, stacked with the first tab and connected with the second elastic piece, wherein the second tab is configured to extend from the first side of the clip plate to the second side of the clip plate through the opening, or the second tab is located at the first side of the clip plate;
wherein at least one of the second elastic piece and the second tab comprises a second arched part.

16. The valve repair device according to claim 1, wherein the first outer clip arm, the second outer clip arm and the distal end part are integral structure, so that the outer clip element has a U-shaped structure.

17. The valve repair device according to claim 1, wherein at least one outer clip arm of the first outer clip arm and the second outer clip arm has a main surface and a thickness perpendicular to the main surface, and the thickness is set to be gradually increased in a direction from the proximal end to the distal end of the at least one outer clip arm.

18. The valve repair device according to claim 1, further comprising:
an outer frame assembly, comprising a first outer frame and a second outer frame, wherein the first outer frame and the second outer frame are pivotally connected to two opposite sides of the distal end part of the outer clip element respectively, so that the first outer frame or the second outer frame is bent into a petal-shaped structure to cover the first outer clip arm and the second outer clip arm, respectively, in a closed state of the outer frame assembly.

19. The valve repair device according to claim 18, wherein each of the first outer frame and the second outer frame comprises:
a pivot part, configured for pivotally connecting with the distal end part, and
a frame structure, extending from the pivot part towards the second hinge structure and has a gradually increased width.

20. The valve repair device according to claim 18, further comprising:
a cover, comprising:
a first covering portion that covers the spacer element, the first clip piece and the second clip piece; and
a second covering portion that covers the first inner clip arm, the second inner clip arm, the first outer clip arm, the second outer clip arm, the distal end part, the first outer frame and the second outer frame.
